(19) United States
(12) Patent Application Publication
(10) Pub. No.: US 2026/0233014 A1
(43) Pub. Date: Aug. 13, 2026

(54) PROCEDURES FOR ASSISTING GUIDANCE OF A PACING ELECTRODE

(71) Applicant: Libra Science Ltd., Road Town (VG)

(72) Inventor: Shlomo BEN-HAIM, Milan (IT)

(73) Assignee: Libra Science Ltd., Road Town (VG)

(21) Appl. No.: 19/536,357

(22) Filed: Feb. 11, 2026

Related U.S. Application Data
(60) Provisional application No. 63/756,887, filed on Feb. 11, 2025.

(51) Int. Cl. A61N 1/368; A61B 5/00; A61B 5/339; A61N 1/05; A61N 1/362

(52) U.S. Cl. CPC A61N 1/3684; A61B 5/339; A61B 5/7264; A61N 1/056; A61N 1/3627

(57) ABSTRACT

A method of a machine-assisted cardiac pacing electrode placement procedure in a patient in need thereof is provided. The method comprising identifying a target location using a 3D data set of a heart of the patient, wherein the target location is identified based on anatomical landmarks, the 3D data set having been acquired using data collection from outside the heart; and providing machine-assisted guidance of the pacing electrode to the target location. Also provided are apparatus and systems for executing the method or parts thereof.

### RELATED APPLICATIONS

This application claims the benefit of priority under 35 USC § 119(e) of U.S. Provisional Patent Application No. 63/756,887 filed on Feb. 11, 2025, the contents of which are incorporated by reference as if fully set forth herein in their entirety.

The contents of the following applications are also incorporated herein by reference in their entirety:
PCT Publication No. WO/2025/032470;
PCT Application No. PCT/IB2025/059358.

These applications also include methods and apparatus which may be used for carrying out some acts in accordance with some embodiments of the invention.

### FIELD AND BACKGROUND OF THE INVENTION

The present invention, in some embodiments thereof, relates toa method of assisting guidance of a pacing electrode in a patient in need thereof.

Pacing the heart is an important cardiac treatment and usually requires attaching a pacing electrode to the heart. Identifying the best location to attach such electrode and attaching a lead at such location are not easily done so a common pacing technique is attaching a pacing lead at the apex of the right ventricle near or at the inter-ventricular septum. This is typically not the most physiologically-effective place to pace the heart from.

### RELATED BACKGROUND ART

The article “Deep Learning for Detecting and Locating Myocardial Infarction by Electrocardiogram: A Literature Review” by P. Xiong, S. Ming-Yuen Lee & G. Chan, Front. Cardiovasc. Med., 25 Mar. 2022, Sec. Coronary Artery Disease Volume 9—2022 doi: 10.3389/fcvm.2022.860032 suggests analyzing ECG signals to detect myocardial infraction locations.

The doctoral thesis by Bytyçi, Ibadete. (2021). “The important role of left atrial function parameters in clinical practice”, evaluates the role of left atrial function in clinical practice.

The article “Cardiac Conduction System: Delineation of Anatomic Landmarks With Multidetector CT”, Indian Pacing Electrophysiol J. 2009 November-December; 9(6): 318-333, states: “Major components of the cardiac conduction system including the sinoatrial node (SAN), atrioventricular node (AVN), the His Bundle, and the right and left bundle branches are too small to be directly visualized by multidetector CT (MDCT) given the limited spatial resolution of current scanners. However, the related anatomic landmarks and variants of this system a well as the areas with special interest to electrophysiologists can be reliably demonstrated by MDCT. Some of these structures and landmarks include the right SAN artery, right atrial cavotricuspid isthmus, Koch triangle, AVN artery, interatrial muscle bundles, and pulmonary veins. In addition, MDCT has an imperative role in demarcating potential arrhythmogenic structures. The aim of this review will be to assess the extent at which MDCT can outline the described anatomic landmarks and therefore provide crucial information used in clinical practice.”

U.S. Pat. No. 8,634,896 describes “A method for 3D reconstruction of the positions of a catheter as it is moved within a human body, comprising: (a) ascertaining the 3D position of a point on a catheter for insertion into the body; (b) acquiring fixed-angle, single-plane fluoroscopic image data of the body and catheter; (c) transferring the image data and catheter-point position to a computer; (d) determining 2D image coordinates of the point on the catheter; (e) changing the insertion length of catheter by a measured amount; (f) acquiring additional single-plane fluoroscopic image data of the body and catheter from the same angle, transferring the length change and image data to the computer, and determining image coordinates of the point on the catheter; (g) computing the 3D position of the catheter point; and (h) repeating steps e-g. A 3D model is constructed by assembling the plural 3D positions of the catheter point.”

U.S. Pat. No. 9,986,931 describes “A method for automatically determining the 3D position and orientation of a radio-opaque medical object in a living body using single-plane fluoroscopy comprising capturing a stream of digitized 2D images from a single-plane fluoroscope (10), detecting the image of the medical object in a subset of the digital 2D images, applying pixel-level geometric calculations to measure the medical-object image, applying conical projection and radial elongation corrections (31) to the image measurements, and calculating the 3D position and orientation of the medical object from the corrected 2D image measurements.”

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A method of assisting real time guidance of a pacing electrode placement procedure in a patient in need thereof, the method comprising:
(a) receiving a cardiac 3D data set of said patient by a computer-based guidance system;
(b) identifying a target location by said system using said cardiac 3D data set, said target location being selected according to a desired cardio mechanical parameter for a statistically significant pacing efficacy to generate a geometric analysis of said 3D data set; and
(c) providing in real-time machine-assisted guidance of said pacing electrode placement to said target location based on said geometric analysis and image data obtained during said placement.

Example 2. The method of example 1, comprising receiving a setting for Hierarchical Physiologic Pacing (HPP) on a computer-based guidance system prior to receiving said cardiac 3D data set.

Example 3. A method of a machine-assisted cardiac pacing electrode placement procedure in a patient in need thereof, comprising:
(a) identifying a target location using a 3D data set of a heart of said patient, said target location being selected according to a desired cardio mechanical parameter for a statistically significant pacing efficacy, said 3D data set having been acquired using data collection from outside the heart; and
(b) providing machine-assisted guidance of a tool (e.g., lead of said electrode) to said target location.

Example 4. The method of example 3, wherein said identifying is by a computer-based guidance system.

Example 5. The method of example 3, wherein said identifying is by a human.

Example 6. A method of cardiac pacing electrode placement procedure in patient in need thereof, the method comprising contacting a lead tip of a pacing electrode with a target location, said target location being defined by a geodesic distance between said lead tip to an anterior position of the floor of the membranous septum (LC) and an aerial distance (LE) between said lead tip and left ventricular endocardium, according to a desired cardio mechanical parameter for a statistically significant pacing efficacy.

Example 7. The method of any one of examples 1-2, 4-5, wherein said system receives said desired cardio mechanical parameter for a statistically significant pacing efficacy.

Example 8. The method of any one of examples 1-5, wherein said target location is identified based on anatomical landmarks.

Example 9. The method of example 8, wherein said target location describes a desired position of a lead tip of said electrode with respect to said anatomical landmarks.

Example 10. The method of example 9, wherein said target location is defined by a geodesic distance between said lead tip to an anterior position of the floor of the membranous septum (LC) and an aerial distance (LE) between said lead tip and left ventricular endocardium.

Example 11. The method of example 10, wherein said LC is below 30 mm.

Example 12. The method of any one of examples 10 and 11, wherein said lead tip is not more than 2.5 mm away from the left ventricular endocardium (LE>−2.5 mm).

Example 13. The method of any one of examples 11-12, wherein said desired cardio mechanical parameter for a statistically significant pacing efficacy is dependent on a heart condition of said patient.

Example 14. The method of example 13 wherein said cardio mechanical parameter is LVEF, and as said LVEF becomes more pathological when said LC is selected shorter, optionally—with each 1 mm decrease in distance corresponding to an increase in LVEF by approximately 0.33%.

Example 15. The method of any one of examples 1-12, wherein said patient is diagnosed with heart failure (HF).

Example 16. The method of any one of examples 1-15, wherein said patient is expected to benefit from reducing LVESV or is selected expected to benefit from reducing LVESV.

Example 17. The method of any one of examples 1-15, wherein said patient is expected to benefit from reducing LVEDD or is selected to benefit from reducing LVEDD.

Example 18. The method of any one of examples 1-15, wherein said patient is diagnosed with a conduction disorder, dilated cardiomyopathy, left ventricular dilation, impaired diastolic function, hypertrophic cardiomyopathy and/or post-myocardial infarction.

Example 19. The method of any one of examples 1-15, wherein said patient is diagnosed with HFrEF.

Example 20. The method of any one of examples 1-15, wherein said patient is diagnosed with HFpEF.

Example 21. The method of any one of examples 1-20, wherein said cardio mechanical parameter is selected from the group consisting of LVEF, LVESF, LVEDV and LVEDD.

Example 22. The method of example 21, wherein said cardio mechanical parameter is LVEF and said statistically significant pacing efficacy is defined by an absolute increase in LVEF of at least 10%.

Example 23. The method of example 21, wherein said cardio mechanical parameter is LVEDV and said statistically significant pacing efficacy is defined by an absolute decrease in LVEDV of at least 45 mL.

Example 24. The method of example 21, wherein said cardio mechanical parameter is LVESV and said statistically significant pacing efficacy is defined by an absolute decrease in LVESV of at least 2 mm.

Example 25. The method of example 21, wherein said cardio mechanical parameter is LVEDD and said statistically significant pacing efficacy is defined by an absolute decrease in LVEDD of at least 5 mm.

Example 26. The method of any of examples 1-25, further comprising an operator fixing said electrode at or near said target location and pacing said target location using said electrode.

Example 27. The method of any of examples 1-5 and 7-26, wherein said 3D data set comprises a CT or MRI data set.

Example 28. The method of any one of examples 1-27, comprising generating a procedure plan based on a geometric analysis.

Example 29. The method of example 28, wherein said generating said procedure plan is by an operator.

Example 30. The method of example 28, wherein said generating said procedure plan is by a computer-based system.

Example 31. The method of any of examples 1-5 and 7-27, wherein said guidance comprises instructions for selecting and/or adjusting a guide sheath.

Example 32. The method of any of examples 1-5 and 7-31, wherein said guidance comprises one or more expected images.

Example 33. The method of any of examples 1-5 and 7-32, wherein said guidance comprises one or more views to use for an imager.

Example 34. The method of example 33, wherein said views include a view in which said electrode and said target location lie in a plane of the imager.

Example 35. The method of example 33 or example 34, wherein said views include a view in which said electrode and said target location lie on a same light of sight of said imager.

Example 36. The method of any of examples 1-5 and 7-35, wherein said guidance comprises one or more delivery trajectories.

Example 37. The method of any of examples 1, 3-5 and 7-36, wherein said providing in real-time machine assisted guidance comprises overlaying said target location of said 3D data set on an a live 2D image during the procedure.

Example 38. The method of example 37, wherein said target location is a volume and overlaying comprises an indication of said volume.

Example 39. The method of any one of examples 1-5 and 7-37, wherein said guidance comprises area or point of penetration and penetration angle.

Example 40. The method of any of examples 1-5 and 7-37, wherein said guidance comprises a number of rotations of a lead or delivery system.

Example 41. The method of any of examples 1-5 and 7-40, wherein said guidance comprises a sheath identity, lead identity, pacemaker and/or one or more set of pacing parameters.

Example 42. The method of any of examples 1-5 and 7-40, wherein said guidance comprises one or more instructions or alerts to show during said procedure.

Example 43. The method of any of examples 1-2, 4-5 and 7-42, further comprising supporting planning said procedure on said image data set before said procedure by an operator.

Example 44. The method of any of examples 1-2, 4-5 and 7-42, wherein said receiving said target location comprises simulating an effect of pacing at said target location.

Example 45. The method of example 44, comprising evaluating a normalizing effect of said pacing on cardiac electrical and/or mechanical activity and/or surface ECG and/or electrocardiogram.

Example 46. The method of example 44 or example 45, comprising providing an output of said simulation as part of said guidance.

Example 47. The method of any of examples 44-46, comprising performing selecting and simulating during said procedure.

Example 48. The method of example 47, wherein said selecting comprises simulating a geometric access to said target location.

Example 49. The method of example 48, wherein said simulating a geometric access takes into account limitations on a guide sheath for said electrode.

Example 50. The method of example 49, wherein said simulating a geometric access determines one or more sheath angulations and/or positions.

Example 51. The method of any of examples 48-50, wherein said simulating a geometric access takes into account limitations on a lead for said electrode.

Example 52. The method of any of examples 48-51, wherein said simulating a geometric access takes into account geometric interference between an object carrying said electrode and said heart, after implantation, which interference carries a risk of adverse effects.

Example 53. The method of any of examples 48-52, wherein said simulating a geometric access simulates a path through a ventricular septum.

Example 54. The method of any of examples 1-5 and 7-53, wherein said guidance is generated using a search function which simulates multiple alternative solutions.

Example 55. The method of example 54, wherein said search function generates a plurality of potential targets and/or paths thereto.

Example 56. The method of example 54 or example 55, wherein said search function takes into account a path of a lead through the RV and/or a path within a ventricular septum being made short.

Example 57. The method of any of examples 1-53, wherein said pacing comprises multi-point pacing.

Example 58. The method of example 57, wherein said multi-point pacing is provided using a single lead or leadless pacemaker.

Example 59. The method of example 57, wherein said multi-point pacing is provided using a plurality of leads or leadless pacemakers.

Example 60. The method of any of examples 1-59, comprising guiding an operator during said procedure.

Example 61. The method of example 60, wherein said guiding comprises marking up one or more fluoroscopic image and/or ultrasonic image.

Example 62. The method of example 60 or example 61, wherein said guiding comprises recommending or applying imager angulations.

Example 63. The method of any of examples 60-62, wherein said guiding generating one or more instructions and/or alerts according to a progress of said procedure.

Example 64. The method of any of examples 60-63, wherein said guiding comprises recommending or applying a delivery system geometric adjustment.

Example 65. The method of any of examples 60-64, wherein said guiding comprises reporting a distance in the septum from a tool tip to an LV wall, the distance being along a trajectory and/or to a nearest wall.

Example 66. The method of any of examples 60-65, wherein said image data obtained during said procedure (for example, during HPP procedure) comprises one or more synthetic DRR images or 3D image, showing a location of at least one tool being used during said procedure.

Example 67. The method of example 66, comprising determining said location of at least one tool using a fluoroscopic imager and/or ultrasonic imager.

Example 68. The method of any one of examples 1-67, wherein said cardiac 3D data set is acquired using data collection from outside the heart.

Example 69. The method of any one of examples 1-2, 4-5 7-67, wherein said image data obtained during said placement is by a fluoroscopic imager and/or ultrasonic imager.

Example 70. Apparatus configured to support generating and/or providing guidance according to any of examples 1-67.

Example 71. Apparatus according to example 68, programmed to generate such guidance.

Example 72. Apparatus configured to support providing guidance according to any of examples 58-67.

Example 73. A method of visualization, comprising:
a. registering a target location landmark for a pacing electrode detected at a prior time in a first coordinate set to a second coordinate set, said target location selected according to a desired cardio mechanical parameter for a statistically significant pacing efficacy; and
b. visualizing a live treatment tool together with said target location landmark in said second coordinate set.

Example 74. The method of example 73, wherein registering target location landmark is in a first imaging modality; and said visualizing a live treatment tool is in a second modality.

Example 75. The method of example 73 or example 74, wherein said tool comprises a pacing lead or an ablation tool.

Example 76. The method of any of examples 73-75, comprising also visualizing a treatment path and/or a treatment target, associated with said landmark.

Example 77. A system comprising circuitry configured to carry out the methods of one or more of examples 73-76.

Example 78. A method of a machine-assisted cardiac pacing electrode placement procedure in a patient in need thereof, comprising:
(a) identifying a target location using a 3D data set of a heart of said patient, wherein said target location is identified based on anatomical landmarks, said 3D data set having been acquired using data collection from outside the heart; and
(b) providing machine-assisted guidance of said pacing electrode to said target location.

Example 79. The method of example 78, wherein said target location describes a desired position of a lead tip of said electrode with respect to said anatomical landmarks.

Example 80. The method of example 79, wherein said target location is defined by a geodesic distance between said lead tip to an anterior position of the floor of the membranous septum (LC) and an aerial distance (LE) between said lead tip and left ventricular endocardium.

Example 81. The method of example 80, wherein said LC is below 30 mm.

Example 82. The method of any one of examples 80 and 81, wherein said lead tip is not more than 2.5 mm away from the left ventricular endocardium (LE>−2.5 mm).

Example 83. A method of affirming/validating pacemaker electrode placement in a target location associated with a portion of a conductance system, the method comprising:
(a) contacting an electrode-mounted pacing lead with the target location; (b) stimulating the target location by said electrode; and
(c) determining a time to onset of electric activity following said stimulating, as determined by ECG.

Example 84. The method of example 83, wherein said time to onset is indicative of an electric activity of a muscle mass.

Example 85. The method of example 84, wherein when said time to onset is above a predetermined threshold it is indicative of stimulating said conductance system and when said time to onset is below a predetermined threshold it is indicative of simulating said muscle mass.

Example 86. A method of machine-assisted setting up of a procedure for conduction system pacing in a patient with a heart, comprising:
(a) identifying a conduction system location on an image data set of said heart, said image acquired using data collection from outside the heart; and
(b) providing machine-assisted guidance of a pacemaker electrode placement to a target location associated with a portion of said conduction system in said heart, said target location suitable for capture of said conduction system by pacing with a pacing pulse from said electrode.

Example 87. The method of example 86, wherein the target location is below the AV node with respect to propagation of a sinus rhythm activation in the heart.

Example 88. The method of example 86, wherein the target location is in and/or adjacent an LBB for LBB pacing.

Example 89. The method of example 88, wherein the placement is within a ventricular septum.

Example 90. The method of example 86, wherein the target location is one or more of Bundle of HIS, RBB, AV, Bachmann bundle and Mahaim fibers.

Example 91. The method of any of examples 86-90, further comprising an operator fixing said electrode at or near said target location and pacing said conduction system using said electrode.

Example 92. The method of any of examples 86-91, wherein said electrode is mounted on a pacing lead.

Example 93. The method of any of examples 86-91, wherein said electrode is part of a leadless pacemaker.

Example 94. The method of any of examples 86-93, wherein said image data set comprises a CT or MRI data set.

Example 95. The method of any of examples 86-94, wherein said identifying comprises identifying a section of at least 2 cm long of the conduction system.

Example 96. The method of any of examples 86-95, wherein said identifying comprises identifying a section of at least 6 cm long of the conduction system.

Example 97. The method of any of examples 86-96, wherein said identifying comprises identifying a ventricular conduction system, including one or more of a LBB, RBB, one or more fascicles and Mahaim fibers.

Example 98. The method of any of examples 86-97, wherein said identifying comprises identifying an atrial conduction system, including one or more of an SA node, a Bachmann bundle, a slow pathway and a fast pathway.

Example 99. The method of any of examples 86-98, wherein said guidance comprises instructions for selecting and/or adjusting a guide sheath.

Example 100. The method of any of examples 86-99, wherein said guidance comprises one or more expected images.

Example 101. The method of any of examples 86-100, wherein said guidance comprises one or more views to use for an imager.

Example 102. The method of example 101, wherein said views include a view in which said electrode and said target location lie in a plane of the imager.

Example 103. The method of example 101 or example 102, wherein said views include a view in which said electrode and said target location lie on a same light of sight of said imager.

Example 104. The method of any of examples 86-103, wherein said guidance comprises one or more delivery trajectories.

Example 105. The method of any of examples 86-104, wherein said guidance comprises one or more 3D annotations and wherein said providing comprises overlaying said annotation on an a live 2D image during the procedure.

Example 106. The method of any of examples 86-105, wherein said guidance comprises a number of rotations of a lead or delivery system.

Example 107. The method of any of examples 86-106, wherein said guidance comprises a sheath identity, lead identity, pacemaker and/or one or more set of pacing parameters.

Example 108. The method of any of examples 86-106, wherein said guidance comprises one or more instructions or alerts to show during said procedure.

Example 109. The method of any of examples 86-108, further comprising supporting planning said procedure on said image data set before said procedure.

Example 110. The method of example 109, wherein said planning comprises selecting said target location.

Example 111. The method of example 110, wherein said selecting comprises selecting based on a current or expected problem in conduction in the conduction system.

Example 112. The method of example 110 or example 111, wherein said selecting comprises selecting based on tissue properties at a potential pacing location.

Example 113. The method of any of examples 110-112, wherein said selecting comprises selecting based on surface ECG measurements.

Example 114. The method of any of examples 110-113, wherein said selecting comprises selecting based on expected power needed for pacing capture.

Example 115. The method of any of examples 110-114, wherein said selecting comprises selecting based on a plurality of targets to be captured by said pacing.

Example 116. The method of any of examples 110-115, wherein said selecting comprises simulating an effect of pacing at said target location.

Example 117. The method of example 116, comprising evaluating a normalizing effect of said pacing on cardiac electrical and/or mechanical activity and/or surface ECG and/or electrocardiogram.

Example 118. The method of example 116 or example 117, comprising providing an output of said simulation as part of said guidance.

Example 119. The method of any of examples 116-118, comprising performing selecting and simulating during said procedure.

Example 120. The method of any of examples 110-115, wherein said selecting comprises simulating a geometric access to said target location.

Example 121. The method of example 120, wherein said simulating a geometric access takes into account limitations on a guide sheath for said electrode.

Example 122. The method of example 121, wherein said simulating a geometric access determines one or more sheath angulations and/or positions.

Example 123. The method of any of examples 120-122, wherein said simulating a geometric access takes into account limitations on a lead for said electrode.

Example 124. The method of any of examples 120-123, wherein said simulating a geometric access takes into account geometric interference between an object carrying said electrode and said heart, after implantation, which interference carries a risk of adverse effects.

Example 125. The method of any of examples 120-124, wherein said simulating a geometric access simulates a path through a ventricular septum.

Example 126. The method of any of examples 86-125, wherein said guidance is generated using a search function which simulates multiple alternative solutions.

Example 127. The method of example 126, wherein said search function generates a plurality of potential targets and/or paths thereto.

Example 128. The method of example 126 or example 127, wherein said search function takes into account a path of a lead through the RV and/or a path within a ventricular septum being made short.

Example 129. The method of any of examples 86-125, wherein said conduction system pacing comprises multi-point pacing.

Example 130. The method of example 129, wherein said multi-point pacing is provided using a single lead or leadless pacemaker.

Example 131. The method of example 129, wherein said multi-point pacing is provided using a plurality of leads or leadless pacemakers.

Example 132. The method of any of examples 86-131, comprising guiding an operator during said procedure.

Example 133. The method of example 132, wherein said guiding comprises marking up one or more fluoroscopic image and/or ultrasonic image.

Example 134. The method of example 132 or example 133, wherein said guiding comprises recommending or applying imager angulations.

Example 135. The method of any of examples 132-134, wherein said guiding generating one or more instructions and/or alerts according to a progress of said procedure.

Example 136. The method of any of examples 132-135, wherein said guiding comprises recommending or applying a delivery system geometric adjustment.

Example 137. The method of any of examples 132-136, wherein said guiding comprises reporting a distance in the septum from a tool tip to an LV wall, the distance being along a trajectory and/or to a nearest wall.

Example 138. The method of any of examples 132-137, wherein said guiding comprises generating one or more synthetic DRR images or 3D image, showing a location of at least one tool being used during the procedure.

Example 139. The method of example 138, comprising determining said location of at least one tool using a fluoroscopic imager and/or ultrasonic imager.

Example 140. A method of a machine-assisted cardiac procedure, comprising:
(a) identifying a conduction system location on an image data set of said heart, said image acquired using data collection from outside the heart; and
(b) providing machine-assisted guidance of a tool to a target location associated with said conduction system location.

Example 141. The method of example 140, comprising following said guidance to bring said tool to said target location.

Example 142. The method of example 140 or example 141, wherein said procedure comprises interacting with said conduction system at or adjacent said target location.

Example 143. The method of any of examples 140-142, wherein said procedure comprises avoiding interacting with said conduction system at or adjacent said target location.

Example 144. A method of navigating a tool with a marker on a real or synthetic fluoroscopic image, comprising:
(a) overlaying on the fluoroscopic image at least one anatomical marker annotation having 3D coordinates;
(b) guiding a user to use or showing the user one or both of:
   (i) a first image with the tool marker and the annotation inline and appearing overlapping or near overlapping; and
   (ii) a second image with the tool marker and the annotation in a same plane with no foreshortening.

Example 145. The method of example 144, wherein said overlaying comprises overlaying at two areas or over an area extending at least 3% of a width of the image and wherein said second image shows both areas in a same plane.

Example 146. A method of conduction system pacing, comprising:
(a) contacting a lead at an area of a RBB, so an electrode on the lead can capture the RBB; and
(b) extending said lead or a part thereof to a location in or across a ventricular septum where a second electrode on the lead can capture the LBB.

Example 147. A method of guiding a cardiac procedure:
(a) identifying a target location in a 3D data set of the heart;
(b) automatically determining at least one parameter of a sheath suitable to reach said target location; and
(c) automated providing of instructions and/or guidance a machine or a human with settings for said sheath that reach said location.

Example 148. Apparatus configured to support generating and/or providing guidance according to any of examples 86-131.

Example 149. Apparatus according to example 148, programmed to generate such guidance.

Example 150. Apparatus configured to support providing guidance according to any of examples 132-139.

Example 151. Apparatus for planning a cardiac procedure, comprising:
(a) a conduction system estimator programmed to receive a 3D data set of the heart and generate an estimation of a location of a conduction system having a length of at least 3 cm;
(b) a GUI configured to show a proposed target location and a proposed path for said procedure.

Example 152. Apparatus according to example 151, comprising one or more of a cardiac electrical activity simulator, a geometric simulator and a search engine.

Example 153. Apparatus for supporting a cardiac procedure, comprising:
(a) an overlayer which overlays a 3D annotation on a fluoroscopic image;
(b) a view generator which generates one or both of an imager viewing angle recommendation and one or more synthetic views.

Example 154. A computer-implemented cardiac simulation method, comprising:
(a) receiving a cardiac 3D data set;
(b) receiving a sheath description;
(c) receiving a target location in a heart; and
(d) geometrically simulating said sheath in said cardiac data set to see if the sheath can be used to reach the target.

Example 155. The method of example 154, comprising varying one or both of said target location and said sheath, while searching for a solution to said reaching.

Example 156. The method of example 154 or example 155, wherein said search has an optimization target of normalizing surface ECG.

Example 157. A method of visualization, comprising:
a. registering a conduction system landmark detected at a prior time in a first coordinate set to a second coordinate set; and
b. visualizing a live treatment tool together with said conduction system landmark in said second coordinate set.

Example 158. The method of example 157, wherein registering a conduction system landmark is in a first imaging modality; and said visualizing a live treatment tool is in a second modality.

Example 159. The method of example 157 or example 158, wherein said tool comprises a pacing lead or an ablation tool.

Example 160. The method of any of examples 157-159, comprising also visualizing a treatment path and/or a treatment target, associated with said landmark.

Example 161. A method of visualization, comprising:
a. automatically identifying and measuring at least one dimension of at least one tool or associated anatomy on a fluoroscopic image of the heart; and
b. displaying said dimension.

Example 162. The method of example 161, wherein said displaying is on said fluoroscopic image.

Example 163. The method of example 161 or example 162, wherein said automatically identifying comprises identifying a ventricular septum and displaying a distance associated with a travel of said at least one tool.

Example 164. The method of example 163, comprising displaying a distance not along a trajectory of said at least one tool.

Example 165. A method of visualization, comprising:
a. generating a 2D or 3D image from a 3D image data set of the heart;
b. acquiring an image of a tool using a 2D imager; and
c. displaying said tool on said generated image.

Example 166. A method of registering an object in a fluoroscopy image to a 3D data set, comprising:
(a) mapping the 3D data set to the fluoroscopy image;
(b) identify a contact of said object in the fluoroscopy image with an anatomical constrain of a tissue imaged in the fluoroscopy image;
(c) using the contact information to constrain the possible mappings from the fluoroscopy image back to the 3D image.

Example 167. A computer-implemented method of choosing a cardiac pacing location, comprising:
(a) receiving cardiac conduction system information;
(b) receiving tissue information about potential pacing locations; and
(c) selecting one or both of a pacing location and a pacing type based on a spatial overlap between said conduction system information and said tissue information.

Example 168. The method of example 167, wherein said tissue information comprises one or more of viability, muscle content, scar content and/or fibrosis.

Example 169. The method of example 167 or example 168, wherein said pacing type comprises one or more of CRT, multi-point pacing, conduction system pacing and no pacing.

Example 170. A computer-implemented method of pacing with heart valve replacement, comprising:
(a) analyzing a potential location for a heart valve placement to identify a potential negative interaction with a conduction system of a patient; and
(b) identifying a potentially effective conduction system pacing location downstream of a location of said potential negative interaction.

Example 171. A method of guiding a cardiac procedure, comprising:
(a) receiving a 3D data set of the heart;
(b) acquiring a live image of the heart including at least one interventional tool;
(c) generating one or more 3D views of the heart which include an indication of said interventional tool; and
(d) displaying said one or more views or one or more projections thereof to an operator.

Example 172. The method of example 171, comprising (e) navigating said tool by said user using said displayed one or more views or projections thereof.

Example 173. The method of example 171 or example 172, comprising using said acquired live image for less than 20% of the time that said displayed one or more views are used, during navigating said tool in said heart.

Example 174. A system comprising circuitry configured to carry out the methods of one or more of examples 154-171.

Example 175. An apparatus configured to support machine-assisted cardiac pacing electrode placement procedure in a patient in need thereof according to any of examples 78-82.

Example 176. An apparatus configured for affirming/validating pacemaker electrode placement in a target location associated with a portion of a conductance system according to any one of examples 83-85.

Example 177. Apparatus configured to provide guidance of a pacing electrode, the apparatus comprising:
a memory storing 3D data set of a heart of a patient; and
at least one processor configured for identifying a target location using said 3D data set of said heart of said patient according to a desired cardio mechanical parameter for a statistically significant pacing efficacy.

Example 178. The apparatus of example 177, wherein said memory stores a cardio mechanical parameter of said patient.

Example 179. The apparatus of example 177 or 178, wherein said at least one processor provides machine-assisted guidance of a tool to said target location.

Example 180. The apparatus of example 177, wherein said guidance comprises:
planning a pacing electrode guidance procedure on said 3D image data set before said procedure by an operator;
simulating an effect of pacing at said target location; and/or
simulating a geometric access to said target location.

Example 180. The apparatus of any one of examples 177-180 comprising an imager for collecting said 3D data set.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as planning or guiding a procedure, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a cross-sectional view of a heart showing substantially all parts of a conduction system;

FIG. 2 is a top level flowchart of a method of treating and/or diagnosing a heart, in accordance with some embodiments of the invention;

FIG. 3 is a top level block diagram of a pre-procedure system usable for treating and/or diagnosing a heart, in accordance with some embodiments of the invention;

FIG. 4 is a top level block diagram of a navigation system usable for treating and/or diagnosing a heart, in accordance with some embodiments of the invention;

FIG. 5 is a more detailed flowchart of a method for treating and/or diagnosing a heart, in accordance with some embodiments of the invention;

FIG. 6 is a cross-sectional view of a heart showing a pacemaker lead in a heart and positioned for conduction system pacing, in accordance with some embodiments of the invention;

FIG. 7 is a flowchart of a method for conduction system pacing, in accordance with some embodiments of the invention;

FIGS. 8A-8D are various synthetic views showing a conduction system or portion thereof, potentially for display to a user in accordance with some embodiments of the invention;

FIGS. 9A-9C show access planning for pacing a LBB, in accordance with some embodiments of the invention;

FIG. 9D shows a transverse view, in accordance with some embodiments of the invention;

FIG. 9E shows two charts, relating to conduction system and tissue health, which may be used to select a pacing location, in accordance with some embodiments of the invention;

FIG. 10 shows comparative access direction and locations for several alternatives of LBB pacing, in accordance with some embodiments of the invention;

FIG. 11 is a side cross-sectional schematic view showing the geometric layout of a sheath during delivery of a pacing lead for conduction system pacing, in accordance with some embodiments of the invention;

FIG. 12 is a flowchart of a method for carrying out an operational stage of treating and/or diagnosing a heart, in accordance with some embodiments of the invention;

FIG. 13 is a flowchart of acts carried out during an operational stage of pacemaker installation, in accordance with some embodiments of the invention;

FIG. 14 is a slide view showing a pacing lead in a common plane with desired penetration points and pacing points on opposite sides of a ventricular septum, in accordance with some embodiments of the invention;

FIG. 15 is an axial view showing a view along an axis of a pacing lead and a line connecting a penetration point and a target pacing point on opposite sides of a ventricular septum, in accordance with some embodiments of the invention;

FIG. 16 is a fluoroscopic image showing a lead with a helix extending to an annotation representing the LBB, showing an exemplary pacemaker lead placement according to some embodiments of the invention;

FIG. 17 shows several views of a pacemaker lead positioned relative to an LBB, in accordance with some embodiments of the invention;

FIG. 18A shows a transverse view DRR image showing a tool layout in the RV, in accordance with some embodiments of the invention;

FIGS. 18B-18D are synthetic view sets at various stages of septum penetration, in accordance with some embodiments of the invention;

FIG. 18E shows matching real and synthetic views, in accordance with some embodiments of the invention;

FIG. 19A is a flowchart of a method of bi-directional registration, in accordance with some embodiments of the invention;

FIG. 19B shows a 3D view showing an interventional tool, in accordance with some embodiments of the invention;

FIG. 20A shows a schematic of a sheath, including two bending locations, in accordance with some embodiments of the invention;

FIG. 20B is a picture of a two-location deformable sheath in various deformation states, in accordance with some embodiments of the invention.

FIG. 20C is a schematic showing of a port face for use with a sheath (adjustable or not) in accordance with some embodiments of the invention;

FIG. 20D is a schematic side view of a sheath showing axial markings in accordance with some embodiments of the invention;

FIG. 20E is another schematic side view of a sheath showing an axially extending orientation line in accordance with some embodiments of the invention;

FIG. 20F is a schematic showing of a handle for a sheath showing controllers for two bend locations, in accordance with some embodiments of the invention;

FIG. 21 is a schematic cross-sectional view of a heart showing a tool, such as a sheath, oriented for trans-septal penetration guided according to the methods of some embodiments of the invention;

FIG. 22 shows guide annotations for valve implantation, in accordance with some embodiments of the invention; and

FIG. 23 shows 3D model views of an undesirable interaction between a valve implantation and the conduction system, which may be avoided in accordance with some embodiments of the invention;

FIG. 24 shows visual determination of pacing sites overlaid on Computed Tomography Angiography (CTA) images, generated using methods of some embodiments of the invention;

FIG. 25A is a depiction of a pacing lead measure, according to some embodiments of the invention, which is based on a geodesic distance from a tip of a pacing lead of a pacing electrode to the branching bundle (LC);

FIG. 25B is a depiction of a pacing lead measure, according to some embodiments of the invention, which is based on an aerial distance from a tip of a pacing lead to the left ventricle endocardium (LE);

FIG. 25C is a depiction of a pacing lead measure, according to some embodiments of the invention, which is based on a geodesic distance from the tip of the pacing lead to the end of the septal fascicle (LX);

FIG. 26A is a graph showing distribution of lead tip distance from the conduction system (geodesic measurements, in mm);

FIG. 26B is a graph showing distribution of Lead tip distance from the endocardium (aerial measurements, in mm), based on a retrospective study;

FIG. 27 is a graph showing relationship of relative (%) change and geodesic L-C distance among heart failure categories, based on a retrospective study. Dashed line represents the linear fit for all data;

FIG. 28 is a graph showing distribution of LVEF change across geodesic L-C and aerial L-E measurements, based on a retrospective study. Most of the patients with an aerial lead-endocardium distance of <−2.5 mm did not have a positive LVEF response in 2 years follow-up. Furthermore, participants with a shorter geodesic lead tip to conduction system distance experienced a larger improvement in LVEF. As a result, the so-called ‘responders’ appear to be clustered in the lower right quadrant of the plot. Subgroups are color-coded;

FIG. 29A is a LAO 20 view: red is the left bundle, pink is the proximal aorta silhouette, the ring behind it is the tricuspid annulus lead shown to reach LV endocardium;

FIG. 29B is a LAO 4 view (almost an AP view) where the lead shown to be at the level of the trunk;

FIG. 29C is a LAO 20 view with the following symbols: Tri—tricuspid annulus; Ao—aorta; T—target for HPP—top of the trunk). Lead is shown to reach the LV endocardium in this view;

FIG. 29D is a RAO view, lead shown past the septal fascicle. Note, this is not a parallax free image. If the camera will be rotated to the rarallax free angle, the lead will be sitting on the septal fascicle;

FIG. 29E is a LAO 30 view—lead reaching the LV endocardium;

FIG. 29F is a LAO 35 view, lead is shown to reach LV endocardium and the conduction system trunk;

FIG. 29G is a RAO view of FIG. 29F—lead is shown to be in the distal part of the trunk;

FIG. 30A is a simple RAO view of fluoroscopy;

FIG. 30B is the same view as in FIG. 30A with marked optimal geometric target location (red) and a sub-optimal geometric target location (yellow);

FIG. 30C is the same view as in FIGS. 30A-B with fully simulated LBB.

### DESCRIPTION OF SPECIFIC EMBODIMENTS OF THE INVENTION

The present invention, in some embodiments thereof, relates to performing cardiac procedures using conduction system information and, more particularly, but not exclusively, to using such data for conduction system pacing.

### Overview

Determining the right location for cardiac pacemaker placement is important for ensuring effective heart function and optimizing patient outcomes. Proper lead placement ensures that electrical impulses are delivered to the heart at the correct location, promoting synchronized ventricular contraction and improving cardiac efficiency. Incorrect placement can lead to suboptimal pacing, reduced heart performance, and complications such as arrhythmias or heart failure.

Embodiments of the present invention relate to the identification of a target location for pacing electrode placement which can be used in HPP procedure.

The heart's conduction system is responsible for the timely activation of both the atria and the ventricles. Timely activation refers to the coordinated stimulation of different areas within the ventricles, ensuring that they contract in an optimal sequence, which adapts based on the physiological state of the heart. For instance, the apex of the ventricles is activated before the base, the endocardium is activated earlier than the epicardium, and the papillary muscles are activated before the rest of the myocardium. This sequence of activation is advantageous for efficient cardiac function. Activation begins at the sinoatrial (SA) node, traveling to the atrioventricular (AV) node, and from there, the fibers travel together until they start to branch out (the branching bundle of His), reaching various targets. The conduction system is highly hierarchical, meaning that activation higher up in the chain has a broader effect on the myocardium, leading to more synchronized contractions. Conversely, activation at lower levels of the conduction system results in non-synchronized activation of the myocardium.

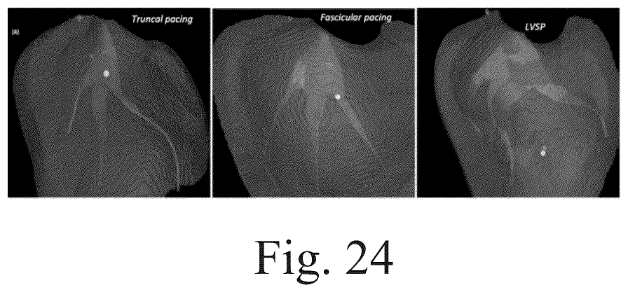

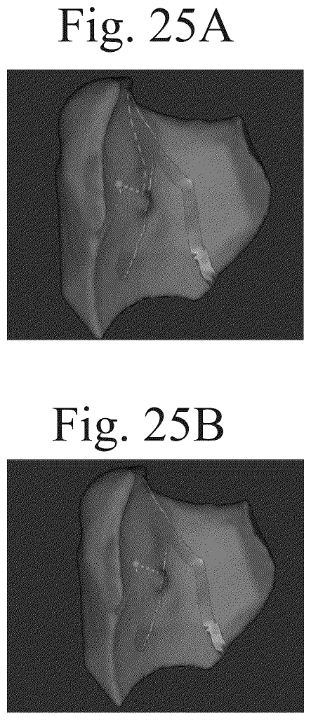

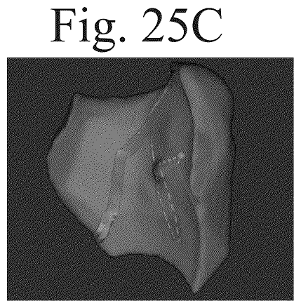

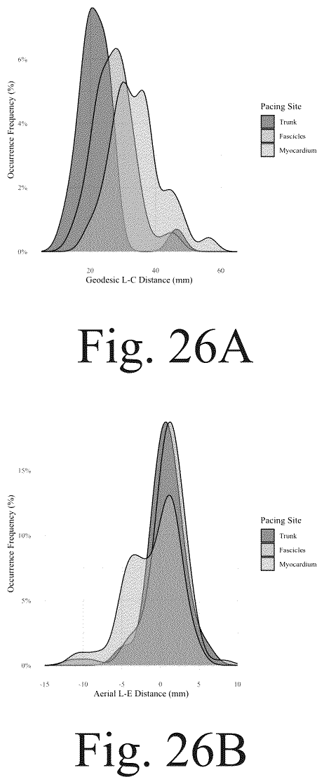

As is described hereinbelow in the Examples section which follows, Computed Tomography Angiography (CTA) was used to classify and evaluate lead placement in patients that underwent conduction system pacing (FIG. 24). Using conduction system simulations and CTA-based visualization, two novel parameters were identified: the geodesic distance between the pacing lead and the floor of the membranous septum and (2) the aerial distance between the pacing lead and the LV endocardium (LE distance), see FIGS. 25A-C and FIGS. 26A-B. These metrics were analyzed alongside baseline and follow-up echocardiographic data to assess their impact on clinical outcomes.

Based on these analyses, the present inventor has realized that the target location for a placement of the pacing electrode can be dictated by a desired cardiomechanical parameter for a statistically significant pacing efficacy.

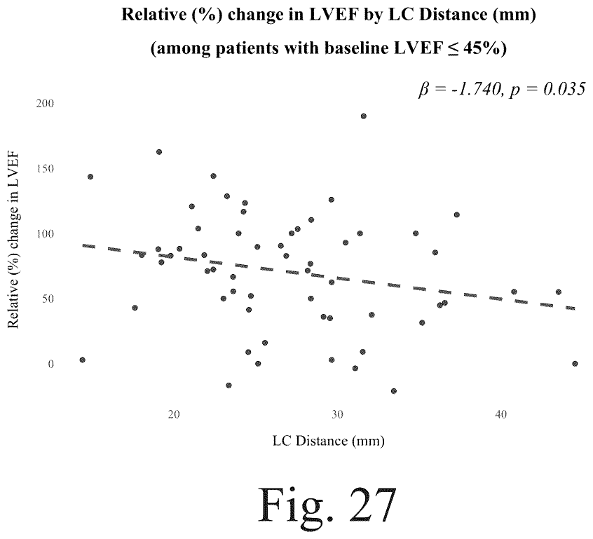

As is shown in the Examples section which follows, for example, LVEF increase is linearly correlated with LC (see FIG. 27). Alternatively or additionally a distance of LE>−2.5 mm had a positive LVEF response in 2 years follow up.

Altogether, these results emphasize the importance of precise pacing lead placement in achieving optimal cardiomechanical responses and advancing the field of physiologic pacing.

The present teachings at least in part thereof relate to a method of assisting real time guidance of a pacing electrode placement in a patient in need thereof.

According to an embodiment, the method is solely machine-based. However, the present teachings also contemplate executing at least a portion of the method by a human operator, such as for example, identification of the target location.

According to some embodiments, identification of the target location is by a computer-based guidance system.

Thus, according to an aspect of the invention there is provided a method of assisting real time guidance of a pacing electrode placement in a patient in need thereof, the method comprising:
(a) receiving a cardiac 3D data set of said patient by a computer-based guidance system;
(b) identifying a target location by said system using said cardiac 3D data set, said target location being selected according to a desired cardiomechanical parameter for a statistically significant pacing efficacy to generate a geometric analysis of said 3D data set; and
(c) providing in real-time machine-assisted guidance of said pacemaker electrode placement to said target location based on said geometric analysis and image data obtained during said placement.

According to an embodiment, the method is solely machine-based. However, the present teachings also contemplate executing at least a portion of the method by a human operator, such as for example, identification of the target location.

Thus, according to some embodiments, identification of the target location is by a computer-based guidance system.

According to other embodiments, identification of the target location is by a human being.

Thus, the present teachings relate to improving the accuracy of pacing electrode placement by providing real-time assistance to operators during the procedure.

According to some embodiments, improving is by at least, 5%, 10%, 15%, 20%, 25%, 30%, 35%, 40%, 45%, 50% or more in at least one cardiac parameter such as selected from heart rate, ventricular conduction, and cardiac output as compared to an absence of said pacemaker tool (prior to treatment).

Generally, the use of pacemakers is contemplated for regulating heart rate, ensuring an adequate rhythm, particularly in cases of bradycardia. According to some embodiments, the cardiac pacemaker can help restore proper conduction within the ventricles, improving synchrony between the heart chambers and/or enhancing overall cardiac output. Additionally, or alternatively, pacemakers can improve atrioventricular (AV) synchrony, ensuring timely activation of the atria and ventricles. According to some embodiments, pacemakers may also enhance left ventricular ejection fraction (LVEF) by improving ventricular synchronization, particularly in patients with heart failure. According to some embodiments, pacemaker can assist in rhythm control by stabilizing abnormal heart rhythms and optimizing pacing thresholds, while also improving the coordination of heart chamber contractions.

According to some embodiments, the “patient in need thereof” is a human or non-human subject who exhibit clinical signs of a cardiac condition that may benefit from pacemaker implantation. The subject can be at any age suffering from a cardiac condition that can benefit from a cardiac pacemaker placement. According to some embodiment, this term encompasses individuals who are at risk to develop the pathology.

As is illustrated hereinbelow and in the Examples section which follows, the present teachings can benefit patients in need for a statistically significant clinical improvement in a cardio mechanical parameter: e.g., LVEF increase, LVEDV decrease, LVESV decrease, LVESD decrease and/or LVEDD decrease. Statistical methods for clinical parameters are known in the art and some are described in the Examples section which follows and are considered an integral part of the specification.

The present inventor has shown that specific cardio mechanical parameters can improve from the proposed method of machine-assisted real time guidance of a pacing electrode. These include but are not limited to the parameters shown in Table 2 below.

As shown, a shorter geodesic L-C distance (closer pacing lead placement) significantly decreases follow-up LVESV (Left Ventricular End-Systolic Volume). This suggests better left ventricular function and more efficient pumping.

Thus, according to some embodiments of the invention the cardiac condition is selected from the group consisting of:
Heart Failure with Reduced Ejection Fraction (HFrEF): This type of heart failure is often associated with impaired ventricular function. Improving LVESV by placing the lead optimally could help restore proper ventricular contraction and improve heart function. Therefore, patients with HFrEF may benefit from shorter geodesic L-C distances;
Conduction Disorders (e.g., Bundle Branch Block): In cases where conduction block delays the normal electrical impulses in the heart, shorter L-C distances could help synchronize the contraction of the left ventricle and improve overall function; and
Dilated Cardiomyopathy: This condition involves an enlarged and weakened left ventricle, which could also see improvements in ventricular function from optimized pacing (shorter L-C distance).
Bradycardia: This condition is characterized by an abnormally slow heart rate, typically defined as fewer than 60 beats per minute in adults:

Alternatively or additionally, it was found that when the aerial L-E distance was greater than −2.5 mm, there was a significant reduction in LVEDD (Left Ventricular End-Diastolic Diameter), which suggests improved left ventricular size or reduction of dilation. This reduction in LVEDD typically means better heart function and may indicate reduced chamber enlargement.

Hence according to some embodiments, the heart condition is selected from the group consisting of:

Heart Failure with Preserved Ejection Fraction (HFpEF): This condition is characterized by normal ejection fraction but impaired filling of the left ventricle. Reducing LVEDD could help restore more efficient ventricular filling, and optimizing pacing lead placement (greater than −2.5 mm aerial L-E distance) might assist with better diastolic function in HFpEF.

Hypertrophic Cardiomyopathy (HCM): In conditions where the heart muscle is abnormally thickened (e.g., HCM), the left ventricle can become less efficient at filling and pumping. The reduction of LVEDD through specific pacing lead placements could help manage symptoms in such patients.

Post-myocardial Infarction (MI): After a heart attack, patients often develop left ventricular dilation (larger LVEDD), which can reduce the heart's efficiency. Reducing LVEDD through optimal pacing lead positioning might help prevent or reverse the enlargement of the left ventricle and improve outcomes.

Bradycardia, as mentioned above.

According to some embodiments, the subject is selected either by the system or by an operator as having a heart condition that may benefit from the present teachings. The subject may be pre-diagnosed with the heart condition, or is diagnosed during the process or is selected to the treatment such as based on pre-existing cardiomechanical parameters.

According to a specific embodiment, the patient is diagnosed with heart failure (HF).

According to a specific embodiment, the desired cardiomechanical parameter for a statistically significant pacing efficacy is dependent on a heart condition of said patient.

According to a specific embodiment, the patient is expected to benefit from reducing LVESV or is selected expected to benefit from reducing LVESV.

According to a specific embodiment, the patient is expected to benefit from reducing LVEDD or is selected to benefit from reducing LVEDD.

According to a specific embodiment, the patient is diagnosed with a conduction disorder, dilated cardiomyopathy, left ventricular dilation, impaired diastolic function, hypertrophic cardiomyopathy and/or post-myocardial infarction.

According to a specific embodiment, the patient is diagnosed with HFrEF.

According to a specific embodiment, the patient is diagnosed with HFpEF.

For cardiomechanical parameters defined as percentages e.g., LVEF, a relative increase by an improvement percentage means the existing value of the parameter is increased by the existing value of the parameter multiplied by the percentage.

An absolute increase means that the existing (baseline) value of the parameter has added to it the improvement percentage.

According to a specific embodiment, the cardiomechanical parameter is LVEF and said statistically significant pacing efficacy is defined by an absolute increase in LVEF of at least 5%.

According to a specific embodiment, the cardiomechanical parameter is LVEF and said statistically significant pacing efficacy is defined by an absolute increase in LVEF of at least 10%.

According to a specific embodiment, the cardiomechanical parameter is LVEF and said statistically significant pacing efficacy is defined by an absolute increase in LVEF of at least 15%.

According to a specific embodiment, the cardiomechanical parameter is LVEF and said statistically significant pacing efficacy is defined by an absolute increase in LVEF of at least 20%.

According to a specific embodiment, the cardiomechanical parameter is LVEF and said statistically significant pacing efficacy is defined by an absolute increase in LVEF of at least 25%.

According to a specific embodiment, the cardiomechanical parameter is LVEF and said statistically significant pacing efficacy is defined by an absolute increase in LVEF of at least 30%.

According to a specific embodiment, the cardiomechanical parameter is LVEF and said statistically significant pacing efficacy is defined by an absolute increase in LVEF of at least 40%.

According to a specific embodiment, the cardiomechanical parameter is LVEF and said statistically significant pacing efficacy is defined by an absolute increase in LVEF of at least 50%.

According to a specific embodiment, the cardiomechanical parameter is LVEDV and said statistically significant pacing efficacy is defined by an absolute decrease in LVEDV of at least 45 mL.

According to a specific embodiment, the cardiomechanical parameter is LVEDV and said statistically significant pacing efficacy is defined by an absolute decrease in LVEDV of at least 50 mL.

According to a specific embodiment, the cardiomechanical parameter is LVEDV and said statistically significant pacing efficacy is defined by an absolute decrease in LVEDV of at least 55 mL.

According to a specific embodiment, the cardiomechanical parameter is LVESV and said statistically significant pacing efficacy is defined by an absolute decrease in LVESV of at least 2 mm.

According to a specific embodiment, the cardiomechanical parameter is LVESV and said statistically significant pacing efficacy is defined by an absolute decrease in LVESV of at least 2.5 mm.

According to a specific embodiment, the cardiomechanical parameter is LVESV and said statistically significant pacing efficacy is defined by an absolute decrease in LVESV of at least 3 mm.

According to a specific embodiment, the said cardiomechanical parameter is LVEDD and said statistically significant pacing efficacy is defined by an absolute decrease in LVEDD of at least 5 mm.

According to a specific embodiment, the said cardiomechanical parameter is LVEDD and said statistically significant pacing efficacy is defined by an absolute decrease in LVEDD of at least 5.5 mm.

According to a specific embodiment, the said cardiomechanical parameter is LVEDD and said statistically significant pacing efficacy is defined by an absolute decrease in LVEDD of at least 6 mm.

According to a specific embodiment, the said cardiomechanical parameter is LVEDD and said statistically significant pacing efficacy is defined by an absolute decrease in LVEDD of at least 6.5 mm.

According to a specific embodiment, the target location and/or conductance system is affirmed using the present teachings, though other methods are also contemplated.

Thus, according to an aspect of the invention there is provided a method of affirming/validating pacemaker electrode placement in a target location associated with a portion of a conductance system, the method comprising:
(a) contacting an electrode-mounted pacing lead with the target location; (b) stimulating the target location by the electrode; and
(c) determining a time to onset of electric activity following the stimulating, as determined by ECG.

According to some embodiment, the time to onset is indicative of an electric activity of a muscle mass.

According to some embodiment, when the time to onset is above a predetermined threshold it is indicative of stimulating the conductance system and when the time to onset is below a predetermined threshold it is indicative of capturing the muscle mass.

According to some embodiments, other time to onset thresholds can be determined to decipher conductance system or another location in the heart.

Methods for measurement time to onset are described in Jastrzabski et al. European Heart Journal (2022) 43, 4161-4173, which is hereby incorporated by reference in its entirety.

According to some embodiments, the method is suitable for placement of a variety of pacemakers as known in the art. These include, without limitation pacing electrodes with leads include single-chamber pacing electrodes, dual-chamber pacing electrodes and biventricular pacing electrodes. Each of these electrodes is connected to a lead and serves different purposes in cardiac pacing systems. Also contemplated are leadless pacemakers (wireless).

According to some embodiments, the method is effected at least in part by a computer-based guidance system (also referred to as an “apparatus”).

According to some embodiments, the method comprises receiving a setting for Hierarchical Physiologic Pacing (HPP) on a computer-based guidance system prior to receiving said cardiac 3D data set.

In other embodiments, the computer-based guidance system is dedicated for HPP.

As mentioned, the computer-based guidance system receives a cardiac 3D data set of said patient by a computer-based guidance system.

Examples of such data include CT data, MRI data or other 3D imaging data.

It will be appreciated that 2D data can also be used for target location identification (e.g., ultrasound).

As mentioned, target location is identified by the system (or an operator as mentioned) using the cardiac 3D data set, according to some embodiments of the invention, the target location being selected according to a desired cardiomechanical parameter for a statistically significant pacing efficacy to generate a geometric analysis of the 3D data set.

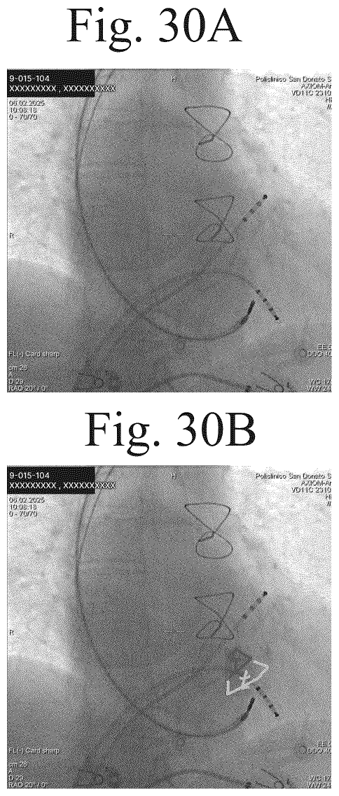

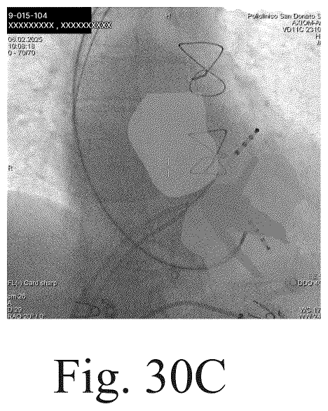

According to a specific embodiment, the target location is a volume, it has spacial dimensions, which can be viewed as a box (see FIGS. 30A-C).

According to a specific embodiment, the target location is selected based on anatomical landmarks, e.g., a specific feature in the heart. Thus, the 3D data set, may be analyzed by an anatomical landmark/gross anatomy features finder to identify anatomical landmarks.

According to a specific embodiment, the target location for lead placement describes a desired position of a pacing lead tip of the electrode with respect to the anatomical landmarks.

According to a specific embodiment, the target location is defined by a geodesic distance between the lead tip to an anterior position of the floor of the membranous septum (LC) and an aerial distance (LE) between the lead tip and left ventricular endocardium.

According to a specific embodiment, a geodesic distance means the shortest path along the curved surface of the heart tissue between two points, such as from a pacing lead tip to a specific target location on the heart. It takes into account the three-dimensional shape of the heart, ensuring that the measurement follows the natural curvature of the heart's surface, rather than a straight line through the tissue.

According to a specific embodiment, an aerial distance in the context of the heart means the straight-line distance between two points, measured along the surface of the heart. This measurement does not follow the curvature of the heart tissue like a geodesic distance but instead represents a direct, linear distance across the heart's surface.

According to a specific embodiment, the LC is below 40 mm.

According to a specific embodiment, the LC is below 35 mm.

According to a specific embodiment, the LC is below 30 mm.

According to a specific embodiment, the LC is below 25 mm.

According to a specific embodiment, the LC is below 20 mm.

According to a specific embodiment, the LC is below 15 mm.

According to a specific embodiment, the LC is above 2 mm (e.g., 5 mm).

According to a specific embodiment, the lead tip is not more than 2.5 mm away from the left ventricular endocardium (LE>−2.5 mm).

According to a specific embodiment, the lead tip is not more than 3 mm away from the left ventricular endocardium (LE>−3 mm).

According to a specific embodiment, the lead tip is not more than 3.5 mm away from the left ventricular endocardium (LE>−3.5 mm).

According to a specific embodiment, the lead tip is not more than 4 mm away from the left ventricular endocardium (LE>−4 mm).

According to a specific embodiment, the lead tip may be in the lumen of the left ventrical. In some embodiments it is not more than 3 mm away from the endocardium.

As mentioned, the selection of the target location for placement is based on a desired cardiomechanical parameter for a statistically significant pacing efficacy or in other terms achieving a specific cardiac function goal.

According to a specific embodiment, the cardiomechanical parameter is selected from the group consisting of Left Ventricular Ejection Fraction (LVEF), Left Ventricular End-Systolic Volume (LVESV), Left Ventricular End-Diastolic Volume (LVEDV), Left Ventricular End-Diastolic Diameter (LVEDD).

The present inventor has shown that the location of the lead tip can be selected based on a desired therapeutic effect.

The present inventor has found that the closer the pacing lead is to the branching bundle of His, the higher is the LVEF response.

According to a specific embodiment, the cardiomechanical parameter is LVEF, and as said LVEF becomes more pathological said LC is selected shorter, optionally—with each 1 mm decrease in distance corresponding to an increase in LVEF by approximately 0.33%.

According to a specific embodiment, the system receives a desired therapeutic effect (e.g., a specific increase in LVEF) and provides the user with guidance for tip placement.

According to some embodiments, a real-time display can be used to show expected results and/or provide guidance to move the lead in the appropriate direction with respect to the target location. For example, guidance for penetrating the right ventricle septum and reaching the left ventricle while assuring that the lead tip is in the endocardium of the left ventrical yet for instance not more than 3 mm away from the endocardium. Guidance can include directional instructions: left, right, up, down. According to some embodiments, the system includes audio instead of or in addition to image audio notifications to alert the user when the lead is in the correct position

According to alternative or additional embodiments, further adjustment may be required and the system will alert at such a situation and provide instructions. Accordingly, in some embodiments, the system provides instructions for adjusting the sheath to facilitate precise lead placement. According to some embodiments, a color based indicated is present such as a red-yellow-green traffic light indicator. Such as indication visually represents the lead's position relative to the target plane or left ventricular (LV) value, where green indicates satisfactory placement, yellow suggests minor adjustments, and red signals a need for repositioning.

According to a specific embodiment, the system receives the desired cardiomechanical parameter for a statistically significant pacing efficacy, based on the patients clinical parameter(s).

In some embodiments, the guidance provides a procedure plan includes one or more of imaging angulation angles, tool angulation settings, expected images, expected in-procedure results, one or more dangers to be avoided and/or one or more alert parameter for an automated system to present. In some embodiments, the plan is generated with the help of simulation and includes both parameters and expected results of following such parameters. Optionally alternatively or additionally, the plan includes one or more alternatives with expected results for such alternatives.

In some embodiments, the plan comprises a target location and the plan defines a volume which encompasses multiple places where pacing is expected to be effective.

In some embodiments, the target location comprises portions which are preferable to other portions of the target location.

In some embodiments, the plan includes a penetration area which indicates locations on the right ventricular septum that can be used as lead penetration points for pathways reaching a desired target location(s).

Planning takes into consideration the angle at which the lead is expected to approach the septum for penetration towards the target volume. During the actual procedure, the actual lead angle, for example, as measured off the x-ray image, may be used to correct the geometry and/or extent of the penetration area marking.

According to some embodiments, the target location is determined and aligned using anatomical landmarks.

According to some embodiments, the conduction system (CS) is not evaluated or delineated.

It will be appreciated that while embodiments of the invention are based on geometric analysis of the target location as described hereinabove for lead tip positioning, they can also use conduction system (CS) estimates/data as described hereinbelow (see in particular CS imaging) to improve the delineation of the target location. For example, using CS estimates to score some parts of the target location volume as being more likely to achieve a desired pacing result.

According to some embodiments, a real-time display can be used to show optimal results (e.g., lead to septum) and/or provide guidance to move the lead in the appropriate direction with respect to the target location. According to some embodiments, the system includes audio notifications to alert the user when the lead is in the correct position or when further adjustment is required. According to some embodiments, the system provides instructions for adjusting the sheath to facilitate precise lead placement. According to some embodiments, a red-yellow-green traffic light indicator is used to visually represent the lead's position relative to the target plane or left ventricular (LV) value, where green indicates optimal placement, yellow suggests minor adjustments, and red signals a need for repositioning.

A broad aspect of some embodiments of the invention relates to treating and/or diagnosing a heart while taking into account the location of parts of the conduction system of the heart. In some embodiments, the location of the conduction system parts is determined by analyzing a pre-operative image, such as a cardiac CT image and not by EP mapping methods. It is noted that some aspects and embodiments described herein, such as mapping tool locations from a fluoroscopic image to a 3D data set and navigation and selection of views, do not need to involve conduction system data.

An aspect of some embodiments of the invention relates to machine-assisted guiding of a cardiac procedure, where the machine-guiding takes into account the locations of parts of the conduction system. In some embodiments, the procedure comprises a cardiac treatment by treatment of a conduction system portion. In some embodiments, the cardiac procedure comprises implanting pacing leads and/or devices. In some embodiments, the cardiac procedure comprises ablation.

A particular feature of some embodiments of the invention is that guiding to a target does not require EP mapping inside the heart. In some embodiments, a small part of the heart may be mapped after guiding thereto, for example, a part with a dimensional extent of up to 4 cm by up to 4 cm.

In some embodiments, CS system detection is based on a pre-operative image, such as a CT image. Optionally, guiding during the procedure uses a fluoroscopic image, optionally annotated based on the 3D image, CS location, procedure guides and/or other data. In some embodiments, guiding during the procedure uses (additionally or alternatively) synthetic images which include information from the procedure, for example, based on data extracted from the fluoroscopic image. In some embodiments, surface ECG data is used for guiding and/or verification.

In some embodiments, the procedure has two stages, a first stage, whereby a 3D image such as a CT is analyzed and a treatment plan is determined (e.g., manually, automatically and/or semi-automatically) and a treatment stage where the plan is carried out and modified as needed. In some embodiments a plan is generated in the first stage and used during the second stage.

In some embodiments, the procedure is CS pacing. Example parts of the CS which may be paced include, the Bachmann bundle, Fast and slow pathways, AV node, bundle of His, LBB, RBB, Mahaim fibers and LBB and RBB fascicles. In each of these, in some embodiments, a particular part can be selected to be paced, for example, a particular part of the Av node or LBB. In some embodiments of the invention, the part is selected with a spatial resolution of better than 20 mm, 10 mm, 7 mm, 5 mm, 2 mm or intermediate precisions. More than one portion of the CS may be paced as part of CS pacing. It is noted that some of these locations, such as the LBB include insulated conduction fibers which generally require more precise targeting if electrification (at a reasonable intensity) is to provide capture.

An aspect of some embodiments of the invention relates to pacing of the atrial conduction system. In some embodiments, a lead is positioned at a part of the atrial conduction system, for example, an SA node, a Bachmann bundle and/or a slow conduction pathway extending from the SA node and/or other internodal pathways.

In some embodiments, atrial pacing is provided together with ventricular pacing, optionally ventricular conduction system pacing. In some embodiments, the timing of atrial pacing is adjusted relative to ventricular pacing, for example, to increase an “atrial kick”, an improvement in cardiac output (on the left and/or right side) due to better loading of the ventricle with blood and/or to help reduce or prevent arrhythmias.

An aspect of some embodiments of the invention relates to multipoint pacing. It is noted that accurate placement with respect to the conduction system of a pacing lead, can be enabling for multi-point pacing of various types. In one type of multi-point pacing, two leads are used and as each has a high probability of pacing the conduction system (or other targeted part of the heart) as intended, the overall success rate remains clinically satisfactory, for example, being above 80%.

In some embodiments, multi-point pacing is provided by a single lead. In some embodiments, the lead is positioned so that an electric field generated by electrode(s) thereon reaches two areas to be paced, for example, the AV node and the bundle of HIS.

In some embodiments, the lead includes two (or more) spaced apart electrodes, and the lead is inserted into tissue so one electrode is at one paced location, for example, an LBB and the other electrode is at a different position, for example, an RBB. In some embodiments, the insertion into tissue is along a trajectory selected during a planning stage and/or during an operation itself.

An aspect of some embodiments of the invention relates to using conduction system data to plan simultaneous pacing of both left and right bundle branches (or other locations on opposite sides of a tissue, such as a septum). In some embodiments, a first step is identifying locations on the branches to be paced. This may be based, for example, on identifying a possible location of a block (to suggest which pacing locations are unblocked) and/or on a portion of each ventricle to be directly paced. Next, if a single lead is used, a trajectory is optionally determined that includes a satisfactory (or better) location on each bundle. Such trajectory may depend, for example, on the distance between the points, any important blood vessels along the trajectory and/or directions possible due to cardiac geometry, lead and/or delivery system limitations and/or avoiding of scar tissue for path and/or pacing locations. Optionally, more than one trajectory is designed and/or the trajectory is reselected during a procedure, based on potential issues with the planned trajectory.

In some embodiments, in procedure, a lead is aimed to have a direction along the trajectory and then a part of the lead is extended (or the lead itself inserted) to the target. In some embodiments, the lead is selected according to the planned trajectory and/or the planned trajectory is selected according to the lead, e.g., inter-electrode distance. In some embodiments, the lead has a screw or helical tip electrode, and the number of rotations needed to reach the target on the far side of the septum is planned and counted. In some embodiments, while penetrating guiding is shown, for example, one or more of—a distance to go, a closest distance to the far surface of the septum, a trajectory and a transverse view or other view, which shows a side, non-parallax view of the trajectory.

An aspect of some embodiments of the invention relates to pacemaker implantation for valve implantations, for example aortic and/or tricuspid implantation. The process of valve implantation can damage the conduction systems, and in some cases, this cannot be avoided. The risk and/or location of such damage is optionally determinable during a planning phase. In some embodiments, based on location of damage, a pacing lead is implanted below a block or damage caused (or expected to be caused) by the valve implant (or other structural heart intervention). In some embodiments, the pacing is within 29 mm, 10 mm, 5 mm or smaller or intermediate distances from such expected location of damage.

An aspect of some embodiments of the invention relates to a procedure planning phase of a cardiac intervention, in which the planning takes into account a location of at least a part of a cardiac conduction system. Optionally the planning takes into account additional information, such as one or more of tissue viability (e.g., scar tissue), ischemia, geometry and location of important blood vessels and/or nerves.

In some embodiments, the planning includes a simulation, for example, a functional simulation and/or a geometric simulation. The simulation may be, for example, for one beat, for part of the heart and/or for a series of beats and/or all of the heart.

In some embodiments, the functional simulation comprises simulating the effect on electrical activity (e.g., action potential propagation, surface ECG, intracardiac electrogram and/or cardio-vectorgram) and/or mechanical activity (e.g., muscle contraction, contraction wave, intracardiac pressures, stroke volume and/or cardiac output). ISE the simulation includes pacemaker parameters and/or logic.

In some embodiments, a geometric simulation includes simulating one or more of possible access paths and how a lead (or implant such as leadless pacemaker) will lie after implantation.

In some embodiments a simulation includes a search function whereby one or more parameters are varied and for each parameter set variation a score or result may be collected. This may be used, for example, to display a map or range of possibilities and/or find one or more working solution. This search may be wholly automatic, for example, or manual or semi-automatic.

In some embodiments the search is used to find solutions for the procedure which normalize (e.g., vary by less than 30%, 20%, 10% or intermediate percentages relative to a baseline value) one or more cardiac-related parameters, such as systolic duration, cardiac output and surface ECG (e.g., time-aligned traces vary by average intensity by less than 30%).

An aspect of some embodiments of the invention relates to using conduction system data and tissue viability data to decide on pacing type and/or location. In some embodiments, tissue with low viability is assumed to be unsuitable as a target for pacing, as a location to place a pacing lead and/or to be interfering with any conduction system portions therein.

In some embodiments, such data is used to select a location for conduction system pacing, for example, below a blocked section of the conduction system and/or above an unblocked section thereof.

In some embodiments, such data is used to evaluate if CRT (e.g., with an electrode in the free wall of the LV, for example, in a coronary's unis or branch thereof) is a suitable pacing regime (e.g., that electrodes can be expected to pace tissue as desired. Optionally alternatively or additionally, such data is used to determine which type of pacing might be expected to be more effective-CS pacing or CRT, or perhaps, neither is expected to be satisfactory and/or clinically worthwhile to attempt.

In some embodiments, viability data is collected from CT data or from nuclear imaging. Optionally conduction system data is collected by analyzing a structural image, such as a CT image, optionally from the same CT data set used for collecting viability data. This may help reduce alignment errors. OP this same image is further used for planning and/or for guiding a procedure, potentially further reducing errors due to misalignment between data sources.

An aspect of some embodiments of the invention relates to personalized pacing in which a location of pacing is selected and executed with a sufficient precision to provide personalized pacing. In some embodiments, the personalized pacing comprises conduction system pacing. In some embodiments the personalized pacing comprises selecting (during a pre-operative stage) and achieving (during an operative stage) a pacing location which is not inhibited by scar tissue or other non-responsive tissue and which can be expected to contract and/or propagate contraction according to said pre-planning and a patient's needs.

An aspect of some embodiment of the invention relates to a pacemaker modified for use with conduction system pacing. In some embodiments, the pacemaker is preprogrammed with one or more parameter sets or ranges that are calibrated for a particular patient, the patient's conduction system, potential failure modes of the implantation (such as missing part or all of the conduction system) and/or optionally the actual location of lead implantation.

In some embodiments the pacemaker is designed, e.g., by programming and including a suitable switching circuitry, to work with a multi-electrode lead so that the specific electrodes to electrify are selected after implantation and based on a relative actual position of the leads and parts of the conduction system

An aspect of some embodiments of the invention relates to a procedure plan for a cardiac procedure. In some embodiments the procedure plan includes one or more of imaging angulation angles, tool angulation settings, expected images, expected in-procedure results, one or more dangers to be avoided and/or one or more alert parameter for an automated system to present. In some embodiments, the plan is generated with the help of simulation and includes both parameters and expected results of following such parameters. Optionally alternatively or additionally, the plan includes one or more alternatives with expected results for such alternatives.

In some embodiments, the plan relates to one or more specific tools, for example a lead and/or a sheath and images and/or guidance based on using such specific tools.

An aspect of some embodiments of the invention relates to using a sheath for guiding interventional tools and/or implants. In some embodiments, the sheath is a cardiac sheath and includes two or more bending locations, though two may be sufficient and/or preferable in some cases. In some embodiments, a plan for a procedure or a suggestion made by a computer or another during a procedure defines these bending angles and optionally axial positioning of the sheath, so as to guide a tool to a desired target area. In some embodiments, a system is provided which translates a cardiac coordinate, for example, a location on a wall into sheath settings.

In some embodiments, a cardiac coordinate includes a plurality of locations, such as two and/or a trajectory and sheath settings are suggested which will result in the sheath aiming along such a trajectory.

An aspect of some embodiments of the invention relates to parallel views in fluoroscopy, in which views objects of interest may be foreshortened. In some embodiments a navigation system is used which detects such foreshortening and alerts a user and/or suggests imaging angles which prevent the parallax and foreshortening. In some embodiments, the system recommends viewing angles where there is no foreshortening if the tools are correctly arranged, as per plan.

In some embodiments of the invention, the system generates a synthetic view in which the foreshortening is corrected, based on the foreshortened view.

In some embodiments, two views are shown, a first view in which a tool and/or its trajectory lie flat, and a second view aligned along an axis of the tool or trajectory thereof.

An aspect of some embodiments of the invention relates to an augmented fluoroscopy image showing annotations generated based on a 3D data set such as a CT. In some embodiments, the image includes both a tool, as imaged, and annotations, for example, of anatomical features that are not directly delineated on the image (e.g., conduction system) and/or of guides for a procedure, such as target locations.

In some embodiments, a user navigates using such an image, in which the user can see, for example, two points of a tool and one or more target indications. In some embodiments, one or both of two images are shown, a first image in which the tool and/or trajectory are aligned with the target(s). A second image is shown along the axis of the tool. In use, the first image shows distances and if the tool is in a correct distance and/or trajectory. In a “correct” situation, all the markers may lie along a single line. The second image, when in a “correct” position, optionally shows all the markers co-located.

It is noted that if the annotations includes two markers, then the alignment method can be used to guide a tool and/or verify that the tool is oriented correctly and optionally positioned correctly as well.

In some embodiments, one or both of the images is synthetically created, but includes both a tool indication and the annotations.

An aspect of some embodiments of the invention relates to mapping from a fluoroscopic image to a CT data set (or other 3D data set) and optionally back to a 2D image such as a fluoroscopic image. In some embodiments, the 2D image has a marking or an object not found in the CT image. Optionally the object's location in 3D space is deducted from the fluoroscopic image with the help of a physical constraint, such as contact between the object and an anatomical structure (which can be detected in various ways, for example, as described herein, and for example, by detecting a change in the shape of the tool when it contacts tissue). In some embodiments of the invention, the system includes a database or a programmed ML system with a set of typical layouts of a tool in the heart. This may be used to deduce the layout, for example, by selecting a closest layout or by interpolation of by ML generated output trained on such layouts. Optionally, an indication of the object position, or an annotation relative to such position, such as a reference location thereon or a current or future interaction of the object or a geometry thereof (e.g., a trajectory) with the anatomy is shown on the 3D image or a derivative thereof. In some embodiments of the invention, the reference location of the tool used for annotation is not on the tool itself, but tracks the tool position and/or orientation. Knowledge of the tool position may be used to calculate the location of this annotation and show it.

In some embodiments of the invention, the 2D image is used to generate a theoretical space of possible locations of the tool and the contact is used to contract the space. Optionally or additionally, anatomical constraints and/or limits on how the device might fit in the body, for example, the heart are used for constraining the set of possible positions.

In some embodiments, the 3D data set is shown as a synthetic two-dimensional image, for example, a simulation of a fluoroscopic image. It is noted that such images may also be shown during a preoperational stage or during operation, when the tool is not yet in the visualization space, but plan for such tool can be shown.

Such mapping may be especially useful for conduction system-related cardiac procedures. First, it allows one to see the conduction system (and/or other annotations) and a tool on the same image. Second, it allows a synthetic view/image to be created, allowing a physician to avoid moving the fluoroscopic imager, while still seeing the tool and target form a desired view. In some embodiments, a plurality of views is shown, for example views perpendicular to each other and/or which align a tool with a target or other marker.

In some embodiments, the view is shown from a point of view of the tool.

Optionally alternatively or additionally, as the tool is mapped back to the 3D data set, a physician can optionally perform the procedure in 3D space, possibly with a 3D imager, the fluoroscope may be relegated for data collection and/or verification, for at least 20%, 30%, 40%, 60% or more of a duration of the procedure where the tool is in the heart.

Optionally alternatively or additionally to a 3D view or a 2D synthetic fluoroscopic view a simplified view is shown, for example, based on segmentation of the CT data and/or the fluoroscopic data. In some embodiments, the view is a wireframe view. In some embodiments of the invention, the coordinates used to show the images are coordinates of the ultrasonic system (e.g., if any, e.g., a trans-esophagus imager), with one axis being the LV axis and the other axes defining the base of the LV. This has the potential advantage of invariance over changes in the cardiac orientation.

An aspect of some embodiments of the invention relates to showing an operator a simulated fluoroscopic image from a view which is physically difficult or impossible to obtain. In particular, a transverse view image may be provided. Such image may be useful when showing how a lead is penetrating a ventricular septum. In some embodiments multiple views are shown simultaneously, optionally with a “real” fluoroscopic view.

It is noted that in general, each of these aspects may be practiced separately without other aspects. In implantation, for example, according to embodiments described below, features not relating to a particular aspect being practiced may be omitted. For example, geometric simulation need not use conduction system detection and 3D navigation need not show annotations of the conduction system.

It is noted that the terms “physician”, “operator” and “user” are used herein substantially interchangeable and relate to a person operating and/or receiving guidance form the system, which are typically physicians but may be technicians or other operators. Also, while CT data is used for some embodiments of the invention, it is noted that CT data can be replaced by MRI data or other 3D imaging data, at least for some embodiments.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

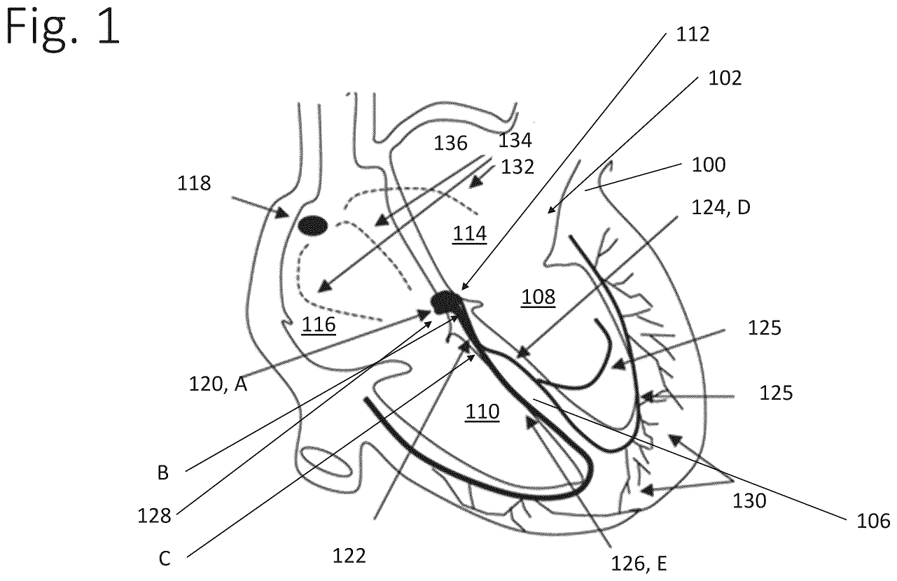

FIG. 1 is a front cross-sectional view of a patient (not shown) 104 having a heart 100 with a conduction system 102, for diagnosis and/or treatment in accordance with some embodiments of the invention. In particular, the heart includes a septum 106, a left ventricle 108 and a right ventricle 110. Also indicated are a membranous septum 112, left atria 114 and right atria 116. As to the conduction system, indicated are an SA node 118, an AV node 120, a bundle of His 122, a LBB 124 (optionally including fascicles 125), a RBB 126, Purkinje fibers 130, fibers of Mahaim 128, Bachmann's bundle 132, a fast pathway 134 and a slow pathway 136. In view of the process for marking them, the following letter annotation is used as well—AV node: A, Penetrating bundle: B, Branching bundle: C, LBB: D, RBB: E. The letters may be used to designate, for example, the start, middle or end of such conduction system component, along the conduction pathway.

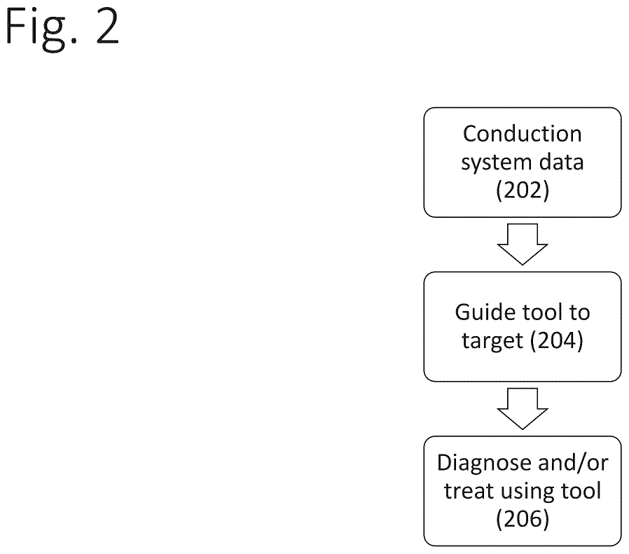

FIG. 2 is a flowchart of a method 200 of diagnosing and/or treating such heart 100, taking into account conduction system 102, in accordance with some embodiments of the invention.

At 202, data indicating the layout of at least part of conduction system 102 of heart 100 are provided. While several ways of such providing are envisioned, in a particular set of embodiments, a cardiac CT is analyzed to generate this layout. This cardiac CT is typically acquired and/or analyzed before active intervention with the body of patient 104. In some embodiments such acquisition and/or analysis is during intervention, such as during catheterization.

At 204, a tool, such as a catheter is guided in and/or to heart 100 to a location selected (e.g., targeted) according to the provided conduction system layout.

At 206, optionally, the guided tool or other tool is used to diagnose the patient and/or treat the patient.

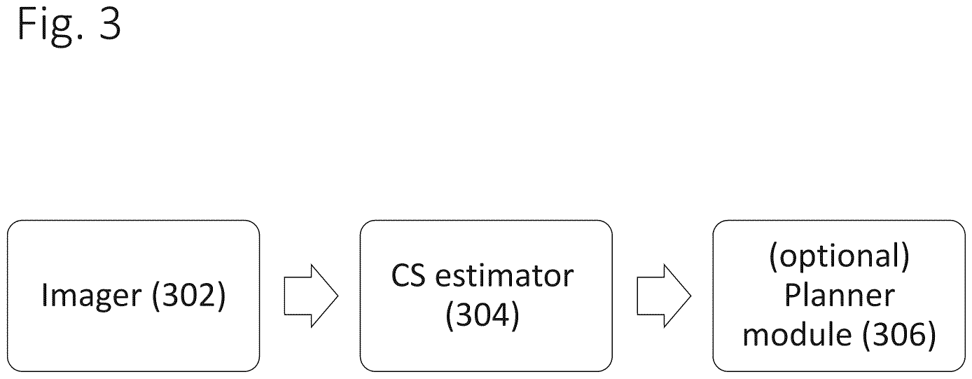

FIG. 3 is a top level block diagram of a pre-procedure system 300 which may be used for CT image acquisition and/or analysis to provide the data of act 202, in accordance with some embodiments of the invention. It should be noted that other apparatus may be used to provide such data, for example, as described below. In some embodiments of the invention, system 300 may also be used for planning. It is noted that such planning may be beneficial, for example, in generating guidance for an actual procedure. However, such planning prior to a procedure is optional and may instead (if at all) be carried out during a procedure.

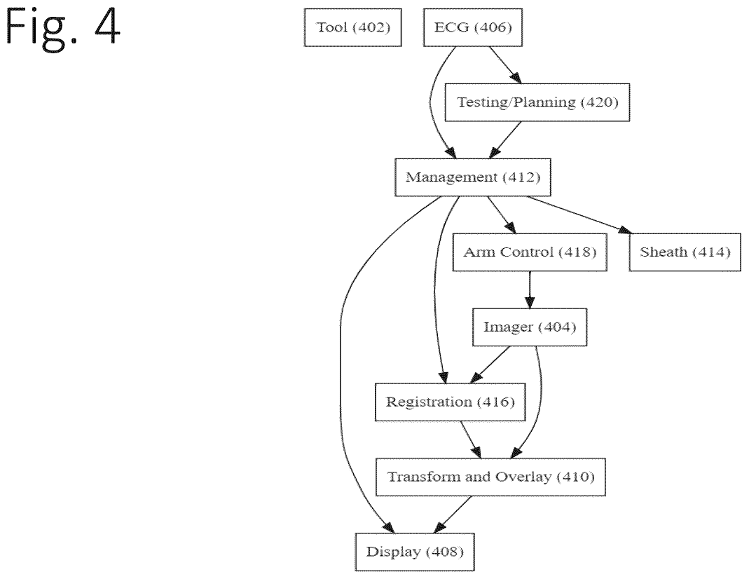

FIG. 4 is a top level block diagram of a system 400 which may be used for said guiding during act 204 in accordance with some embodiments of the invention.

Referring first to FIG. 3, a CT imager 302 is shown as an example of an anatomical imager. Other imager types may be used as well. The image acquired by imager 302, for example, a 3D data set, may be analyzed by an anatomical landmark/gross anatomy features finder 302 to identify anatomical landmarks. After anatomical landmark finding, a conduction system estimator 304 may be used to identify the location of at least a portion of conduction system 102 on the 3D image. In some embodiments of the invention, modules 302 and 304 are combined into a single module, for example, a machine learning model which maps anatomical images to conduction system layouts. The output of estimator 304 may include conduction system layout coordinates for use before and/or during a procedure.

An optionally planner module 306 (which may include a simulator) may be used to help plan a diagnostic and/or therapeutic procedure, based on conduction system 102. Module 306 may output plan data, which can include, for example, instructions to machines and/or humans.

Referring now to FIG. 4, a tool 402, for example, a catheter is guided to a target location, for example a particular location in conduction system 102. Tool 402 and/or heart 100 may be tracked during the procedure, for example, using an optional imager 404 (e.g., x-ray fluoroscopy and/or ultrasound). Data may be collected from the heart, for example, using an ECG 406. A registration module 416 is optionally used for calculating registration of a fluoroscopic (or other in-procedure) image to the conduction system data and/or CT image. Guidance to the user and/or portions of the conduction system may be presented on an optional display 408, for example, as markup on a fluoroscopy image, for example, as a color overlay. Optionally, a transform and overlay module 410 is used to calculate position and/or generate an overlay for display 408. Optionally or additionally, a management module 412 is used to help guide the procedure, for example, by generating indications and/or alerts and/or responding to user requests. Optionally, an arm control module 418 controls a position of a C-arm or other imager according to said guidance. Also shown is an optional guide sheath 414 which is optionally manufactured, controlled, and/or manually manipulable to help guide tool 402 to the target location. In some embodiments of the invention, system 400 may also be used for planning. A testing/planning module 420 is optionally provided for guiding testing of an ongoing procedure and/or in-procedure planning and calculations relating to the procedure itself, for example, paths and/or simulation of effects. In this application, the term “system” is used to describe system 300 and/or system 400 (which can be implemented, for example, as a single configuration, two separate subsystem and/or as multiple components, possibly some remote). In some embodiments of the invention, processes such as image analysis, simulation, guidance generation, image generation and/or guidance providing are performed by the system. In some embodiments, the performance is semi-automatic—with a user requesting an action and/or in response to user action and/or with a user providing significant input (e.g., for locating a conduction system or suggesting a treatment). However, automated methods are described as well (e.g., for conduction system data extraction, target selection and/or plan evaluation) and may be used in some embodiments of the invention.

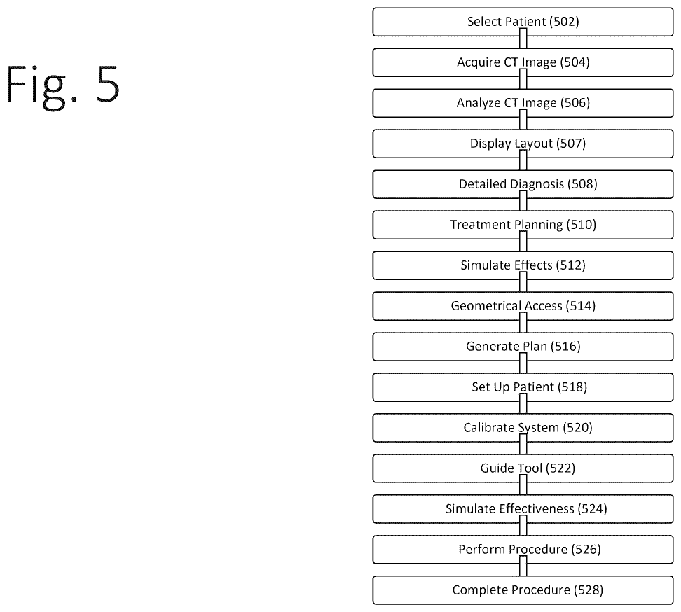

Referring now to FIG. 5, which is a more detailed flowchart 500 of a method of diagnosing and/or treating patient 100 (e.g., as an example of FIG. 2), in accordance with some embodiments of the invention. The acts in this flowchart will serve as the backbone of a part of the detailed description of this disclosure, with each act described in greater detail in separate sections below. Details in one section may be applied in other sections as well.

Flowchart 500 focuses on a particular procedure, that of conduction system pacing, where a pacing lead is positioned to stimulate a part of a conduction system 102, for example, using the methods described herein.

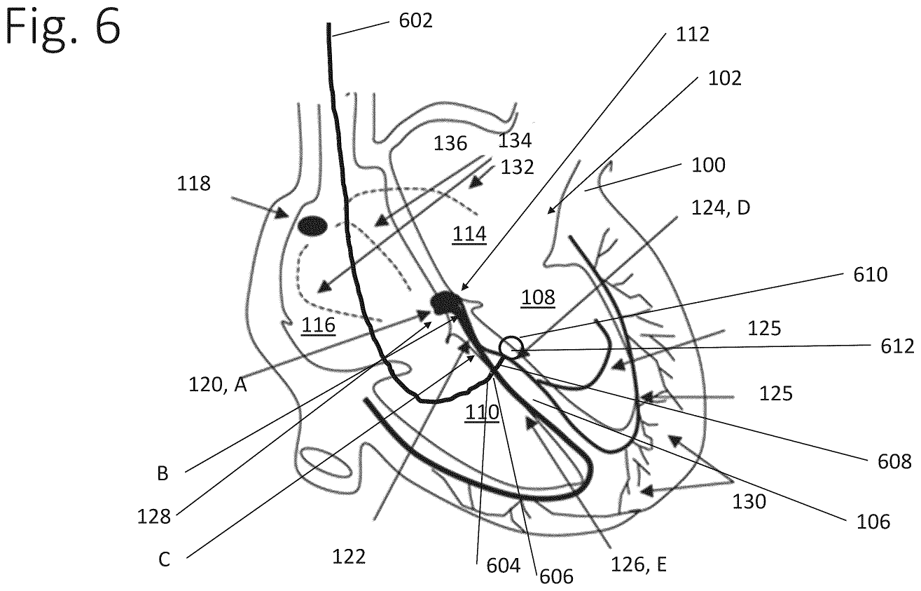

FIG. 6, which is similar to FIG. 1 showing heart 100, is used to illustrate one representative example of conduction system pacing in accordance with some embodiments of the invention. FIG. 6 shows a pacing lead 602, having a tip 604 reaching a surface target 606 and which optionally penetrates a depth 608 into the septum so that an electric field 610 generated thereby reaches a targeted conduction system location 612. More details and alternatives for pacing and/or other procedures are described herein.

Referring now back to FIG. 5, the acts are described briefly and as a prelude for the below, noting that the order of the acts can change, some cats are optional and the contents of the acts can be different.

At 502, a patient is selected, for example, for CS (conduction system) pacing.

At 504, a cardiac CT image (or other anatomical image) is acquired of heart 100.

At 506, the CT image is analyzed to identify a layout of conduction system 102. Optionally, such layout is displayed on the CT image (507).

At 508, a more detailed diagnosis may be performed, for example, combining data from the CT image and ECG data.

At 510, a treatment is planned, for example, determining the surface 606 and/or CS location(s) 612 for pacing heart 100.

At 512, effects of such treatment are optionally simulated and/or treatment changed accordingly.

At 514, geometrical access to surface location 606 and/or CS location 612 are optionally selected and/or simulated optionally resulting in changes in treatment planning and/or target locations.

At 516, a plan for the procedure is optionally generated.

At 518, the patient is set up for treatment, for example, in a cath-lab. It is noted that in some embodiments, one or more of acts 502-516 are done after such setup.

At 520, system 400 is optionally calibrated to actual patient conditions.

At 522, tool 402 is guided to the heart, optionally a sheath, optionally to surface target 606. In some embodiments of the invention, such guiding may include indications shown on a fluoroscopic image and/or machine control of tool 402. Optionally or additionally, such guiding includes one or more alerts and/or warnings. At 524, a simulation of the effectiveness of the treatment may be made based on actual location of tool 402 and/or data measurements, optionally using a simulation.

At 526, the procedure is performed, for example, screwing a pacing lead at surface target 606. Such performance may be under monitoring of system 400.

At 528, the procedure is completed. Optionally, data generated before the procedure and/or collected during the procedure are used to predict the effects on patient 104, which maybe used a monitoring physician to determine effectiveness of treatment and/or suggest follow up treatment.

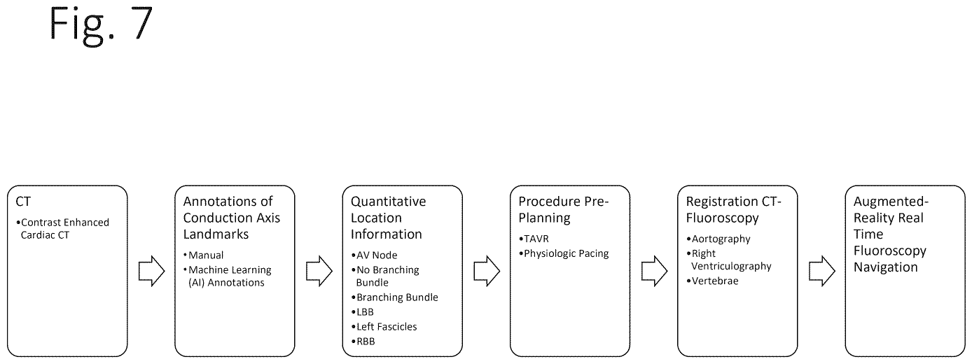

Referring now to FIG. 7 which shows an example flow diagram 700 of a method of diagnosis and/or therapy in accordance with some embodiments of the invention. As can be seen, the system may be divided into two stages with corresponding hardware and/or software. First, at a pre-procedure stage 702, a CT image is acquired (706), various landmarks connected to the conduction system are noted (708) and actual conduction system portion locations may be generated (710). Second, a navigator stage 704 includes a pre-planning process 712 (which may include simulations, for example as described herein), a registration process 714 where the coordinates for the data from the CT is aligned with coordinates of the fluoroscope image (if any, or other image) and finally augmented reality display on a fluoroscopic image to help actual navigation, diagnosis and/or treatment 716. Each such process may use separate computing hardware which can be, for example, in a cath-lab, in a physician office or, at least in part, on a local or remote computing location (e.g., a cloud).

In one example type of implementations, planning is done on a remote system, for example accessed as a cloud. In some cases, the user interface and some functions run locally, for example, to enhance reaction time, but certain features, for example, conduction system detection, searches, simulations, recommendations and/or image generation, are handled remotely.

In-procedure support, for example, registration, image alignment, alerts, measurements, imager control, angulation output and/or overlay generation is optionally performed locally. This may be useful to reduce a risk of a procedure getting stuck due to a data outage. Some functions may be provided remotely, for example suggestions and/or requests for re-planning.

The plan itself may be stored locally and/or remotely.

A potential benefit of remote functionality is ease of updating functionality and/or reacting to potentially serious software problems and/or reduced hardware requirements (e.g., a capital expense). Remote access potentially supports easier protection of secret methods and data. Remote access potentially enables gatekeeping by the remote system which can be used, for example, to limit unauthorized use of the system and/or using metering methods for charging (e.g., counting number of uses and/or patients)

As noted, in some embodiments of the invention, time critical and/or clinically critical (e.g., lifesaving and/or death preventing) information is processed and/or used locally. Information which can bear time delay, requires heavier processing and/or is less clinically critical, may be done by remote.

### Patient Intake

Referring back to FIG. 5, at 502, a patient is selected for a procedure. In some embodiments of the invention, the patient is any patient about to undergo catheterization and/or EP procedures. As noted, in some particular embodiments of the invention, the procedure is a pacing lead implantation procedure. A particular potential benefit of some embodiments of the invention is in the ability to guide an implantation to a desired physical and/or functional location in the heart and patients may be selected to take advantage of such benefits. This can support conduction system pacing. In conduction system pacing a lead is delivered to the heart (e.g., through the right ventricle) with a stimulation electrode located so that it can stimulate a desired part of the conduction system. It is believed that such stimulation results in more physiological and/or effective cardiac contraction, for example, due to timing and sequencing benefits provided by using the native conduction system rather than general cell-to-cell propagation.

Potentially, simulation, for example as described herein, can help guide the selection of an appropriate pacing mode for HF patients or at risk for HF, or bradycardia patients, for example, by comparing location of scar and conduction on the left ventricle myocardium. The presence of a scar suggests a reduced ability to reach conduction system within it and/or reduced probability of muscle recruitment in areas of scar. The selection of mode and/or location of pacing leads can therefore potentially be personalized, in accordance with some embodiments of the invention.

Some particular procedures that can be provided using methods described herein include:
(a) AV node pacing;
(b) His bundle pacing;
(c) pacing of the Left bundle branch (LBB) and/or Right Bundle Branch (RBB);
(d) simultaneous pacing both the LBB and RBB and optionally the AV node, optionally or alternatively with a single lead;
(e) pacing of the Mahaim fibers;
(f) pacing below a conduction system block;
(g) pacing at an arrhythmia foci or part of a circuit or other anti-arrhythmia pacing;
(h) CRT using leads positioned to selectively stimulate the left or right ventricle;
(i) general multipoint pacing with electrode placement with an accuracy of greater than 1 cm;
(j) pacing including any combination of the above;
(k) ablation, for example of the fast pathway, slow pathway, AV node and/or one or more conduction pathways in an atrium and/or ventricle;

It is noted that simultaneous and/or separate pacing of multiple regions using a same lead may require more careful selection of penetration point (e.g., near but not through; or through the RBB) and/or penetration trajectory and/or penetration angle so that there are electrodes positioned correctly to electrify all the desired locations. pacemaker settings may also be different and/or the search space for such setting may need to take into account the desired simulation locations and relative timings therebetween.

In some embodiments of the invention, a pacing lead is used which includes multiple electrodes, allowing for applying a pacing signal at multiple points along the lead, for example, 2, 3, 4 or more points. For example, in a bi-polar each point (or rather pacing capture volume) is defined by two adjacent electrodes, such as a tip or ring electrodes. In some embodiments of the invention, electrodes that are not adjacent may be used. In a uni-polar mode, each electrode defines such a volume. The pacemaker optionally includes switching circuitry for selecting which electrodes/pairs to stimulate when pacing A potential advantage of such a lead is that after implantation, the actual electrodes used for pacing may be varied, for example, to provide pacing of both an LBB and an RBB separately, though with the same lead. Optionally, after the procedure the pacemaker is associated with or programmed with instructions and/or data regarding the leads and/or which pacing settings (including electrode selections) had what effects during the procedure. It is noted that post implantation there may be changes in electrode-tissue interaction for various reasons (e.g., post procedure edema, inflammation and/or fibrosis cap formation), at various times and/or for various lengths of time.

A particular benefit of using some methods described herein for pacing is that pacing parts of the conduction system often require precision in targeting of a tissue which is not easy to image. Methods described herein can be used to locate the relevant part of the conduction system to pace and/or evaluate effectiveness of pacing and/or guide lead implantation to perform such pacing.

### CS Imaging

A CS map (or more generally, data about parts of the conduction, such as location relative to other parts of the heart) may be created in various manners. In some embodiments of the invention, a 3D structural image of the heart is used to generate such CS data.

In some particular embodiments, at 504, a cardiac CT image of the heart is acquired as such a structural image. But other structural images may be used, for example, MRI and ultrasound.

At 506, the structural image (with CT as an example) is analyzed to identify the layout of conduction system 102. As will be explained below, in some embodiments, the conduction system location can be overlaid on a fluoroscopy or ultrasound (or other) image acquired during a procedure.

In some embodiments of the invention, the CT image is analyzed using a CT analysis package that suggests the location of one or more of the following portions of the conduction system, relative to gross anatomical features, for example, relative to valve annuluses, leaflets, septum and/or the membranous septum:
a. SA node
b. Bachman Bundle
c. Slow pathway
d. Fast Pathway
e. AV node
f. His Bundle
g. Branching bundle
   i. Left Bundle Branch (LBB)
   ii. Left Anterior Fascicle (LAF)
   iii. Left Septal Fascicle (LSF)
   iv. Left Posterior Fascicle (LPF)
   v. Right Bundle Branch (RBB)
   vi. Mahaiem Fibers (MF)
h. Left ventricle papillary muscle
i. Right ventricle Papillary muscle

The specific conduction systems portions identified and/or anatomical features identified (and/or later visualized) are optionally selected according to the target procedure.

A potential advantage of using a CT or structural image is that the conduction system can be mapped without catheterization of the heart, especially no left ventricle or left side catheterization.

Another potential advantage of structural based mapping of a conduction system over EP electrical mapping is that structural based mapping can show the location of a blocked conduction system section, which, if suitably paced, may provide a desired conduction. In some embodiments of the invention, the structural image is analyzed to identify dead areas, in which the conduction system may be inoperative. Such areas may be identified, for example, using MRI, nuclear-medicine imaging and/or CT images (e.g., where wash-in and wash-out are analyzed).

In some embodiments of the invention, a CS map includes multiple layers, for example, anatomical-based conduction system location, electrical measurement based data and/or functional data such as perfusion or viability (e.g., from a PET or SPECT image or a contrast CT image). Such added layers may be aligned using the CT image or anatomical data extracted therefrom.

In some embodiments of the invention, the CS data is used to build a conduction model of the heart which is optionally used to predict the effect of electrical stimulation on the propagation of electrical signals in the heart and/or contraction thereof. Optionally, the model is a standard model and the CS data is used as parameters for such a model, such as more exact location of certain pathways or using anatomical data, for example, to model a more exact shape of the heart.

In some embodiments of the invention, the conduction system data is provided using methods described in PCT application IB2024/057540, the disclosure of which is incorporated herein by reference.

In one example embodiment of the invention, the following method is used to identify the conduction system. This method may be applied automatically, manually or semi-automatically, for example. It is noted that some parts may be skipped and some parts of the conduction system unmarked or be marked differently, the phrase “drawing” intended to convey a mathematical equivalent which may also be used for marking up an image. Any of the anatomical landmarks described herein may be identified based on location, shape and/or tissue type by image analysis of a CT data set, for example, using image segmentation methods, optionally using templates. Other methods may be used as well. In some embodiments of the invention, the methods use take into account anatomical structures which are visible, for example, as landmarks on a CT image and/or an MRI image. In some embodiments of the invention, the anatomical structures are selected because they constrain the possible locations for a portion of the conduction system and/or because they guide and/or co-develop with the conduction system during fetal development.
1. Segment patient CT
   a. Identify chambers, vascular system, bones,
2. Mark the tricuspid annulus (e.g., by identifying the plane between the RA and RV)
3. Calculate center lines of the vascular system, e.g., one or more of:
   a. Subclavian
   b. SVC
   c. RA; and
   d. RV
4. Mark one or more of the A B C points of the conduction system:
   a. A—AV Node
   b. B—penetrating bundle; and
   c. C—branching bundle
5. Mark point/area D, in one method:
   a. Draw a line connecting the point C to the LV apex lying on the LV septal wall
   b. Mark point D1 at 1 cm below point C on that line
   c. Mark point D2 at 2 cm below point C on that line
   d. Mark point D3 at 3 cm below point C on that line—this may be repeated for more points D
   e. Optionally place a perpendicular line through points D with a fixed width of 5 mm or 10 mm or any value from 3 to 12 mm.
   f. Optionally place a perpendicular line through points D with a non-fixed, increasing width of, for example, 5 mm for D1 7 mm for D2, with the line widening from a minimum of 3 mm or more to a maximum of 15 mm or less.
6. Mark point/area D, in another method
   a. Draw a line connecting the point C to the LV apex lying on the LV septal wall—this line will be the direction the septal fascicle will be oriented for orienting the placement of the LB template (below)
   b. Draw a line connecting the point C to the base of the anterior papillary muscle in the LV—this line will be the direction of the anterior (superior) fascicle will be oriented for orienting the placement of the LB template (below)
   c. Draw a line connecting the point C to the base of the posterior papillary muscle in the LV—this line will be the direction of the posterior (inferior) fascicle will be oriented for orienting the placement of the LB template (below)
   d. An LB template is scaled to the real patient heart anatomy—the distance from C to LV apex, the width of the LV base based on weighing their ratio to the ratio the template describes, and overlaid on the heart, for example, curved to conform to the surface of the LV wall. In some embodiments of the invention, the LBB estimation or template is urged against the wall as the pathways are extended. For example, first the LBB is positioned against the wall and then the fascicles are positioned against the wall. In one example, an estimation process extends along the conduction pathway, one small part at a time and each part is positioned against the wall and laterally on (or in) the wall.
      In some embodiments of the invention, the conduction pathway is shown narrower and/or shorter than it actually is, for example, 70%, 60%, 40% or smaller or intermediate percentages. This may assist in preventing an operator from aiming to a more peripheral part of the conduction system which, due to intra-personal variations, might not exist at that location. Optionally alternatively or additionally, the showing of the conduction system is shaded or stippled or otherwise indicated to show confidence.
7. Point D may be marked at the lower part of the LBB (before it bifurcate to the fascicles) and may be a useful target for LBB pacing
8. Point E (RBB split from the His bundle toward the RV lumen) may also be noted; points D and E may be useful for dual chamber pacing, for example, using a single lead. In some embodiments of the invention, when two leads are used a safety distance is kept and/or monitored e.g., by the system (e.g., during planning and/or during implantation) between the contact points of the leads where they contact cardiac tissue, for example, 1, 2, 3, 4, 6, 10, 15 mm or smaller or intermediate distances. Alternatively, no safety distance is kept.
9. Optionally parts of the atrial conduction system are marked, for example, as described herein, for example, one or more of the SA node, Bachmann's bundle and intermodal pathways.

Another set of methods method which may be used for identifying the conduction system, together with or instead of other methods herein, comprises:
a. Identifying the Inferoseptal recess, for example, by one or more of
   i. By its differentiated content from the rest of the adjacent tissue (fibrous tissue vs. muscle or blood) as seen by various volumetric imaging system
   ii. By its neighbors
      1. The roof of the inferoseptal recess is formed by an area of fibrous continuity between the leaflets of the mitral and tricuspid valves. This fibrous tissue supports the base of the atrial septum
      2. Anterior/Superior: The aortic leaflet of the mitral valve forms the anterior/superior border
      3. Posterior/Inferior: The septal surface of the left ventricle forms the posterior/inferior border
      4. Right side: The right wall of the recess is formed by fibrous tissue through which the conduction axis (His bundle) typically penetrates
      5. Left side: The left ventricular cavity borders the left side of the recess.
   iii. By an alternative analysis of nearby anatomy:
      1. The inferoseptal recess is bounded on the atrial side by the diverging vestibules of the mitral and tricuspid valves. These vestibules are part of the atrial anatomy and play a role in the function of the atrioventricular valves
      2. The ventricular boundary of the inferoseptal recess is formed by the crest of the muscular ventricular septum. This septum is the thick, muscular wall that separates the left and right ventricles of the heart
      3. At its apex, the inferoseptal recess is marked by the inferior margin of the membranous septum. This septum typically acts as an atrioventricular partition at this level and is often overlaid by a layer of atrial vestibular myocardium. This layer separates the apex of the pyramidal space from the inferoseptal recess within the left ventricle
   iv. And/Or by the following set of neighboring anatomical structures:
      1. Base—The base of the IPS is located on the diaphragmatic surface of the heart. This is the bottom part of the pyramid-shaped space.
      2. Apex—The apex of the IPS is near the central fibrous body of the atrioventricular septum, close to the noncoronary sinus of the aortic root.
      3. Anterior Border—The anterior border is formed by the crest of the interventricular septum.
      4. Posterior Border—The posterior border is continuous with the diaphragmatic surface of the heart at the crossing of the left and right inferior atrioventricular and interatrial grooves.
      5. Lateral Borders—The lateral borders are defined by the two septal atrial walls.
b. Infrapyramidal space may be identified, for example:
   i. By identifying on the volumetric imaging its differentiated content (fat and fibers as compared to surrounding muscle)
      1. For example identifying a low HUE epicardial invagination volume of cardiac CT
      2. For example identifying a high signal intensity on T1 weighted images of cardiac MRI
   ii. And/or based on structure, for example:
      1. Location and Shape
         a. The inferior pyramidal space is a pyramid-shaped fibrofatty structure located on the diaphragmatic (inferior) surface of the heart. It wedges between the four cardiac chambers from the diaphragmatic surface
   2. Its border are:
      a. The apex of the pyramid is located near the central fibrous body of the atrioventricular septum, close to the noncoronary sinus of the aortic root
      b. Inferior: The base of the pyramid is on the diaphragmatic surface of the heart
      c. Formed by the two septal atrial walls
      d. Bounded by the crest of the interventricular septum
      e. The space is continuous with the left and right inferior atrioventricular and interatrial grooves at their intersection on the diaphragmatic surface
c. Using the infero septal recess and the infrapyramidal space identifications, one can locate the patient specific location of the conduction system axis. Using, for example, these rules:
   i. To understand how the infra septal recess and the infra pyramidal space relate to the cardiac conduction system, including the AV node, Bundle of His, and the left and right bundles, one takes into account their anatomical positions and their relationship with the conduction pathways.
   ii. The Infra Septal Recess is significant because it is close to the atrioventricular (AV) node and the Bundle of His
   iii. The Infra Pyramidal Space is closely related to the AV node and the initial part of the Bundle of His.
      1. AV Node—is situated within the atrioventricular septum, near the opening of the coronary sinus, and is part of the triangle of Koch, which is bounded by the tendon of Todaro, the tricuspid valve annulus, and the coronary sinus ostium, The AV node is located just above the infra septal recess, making this recess a landmark for accessing the AV node during procedures as well AV node is also adjacent to the apex infra pyramidal space, which provides a pathway for the AV node to transition into the Bundle of His.
      2. The Bundle of His originates from the AV node and travels through the floor of the membranous part of the interventricular septum
      3. The Bundle of His passes close to the infra septal recess as it descends through the septum. The Bundle of His is closely associated with the infra pyramidal space, particularly at its origin from the AV node. During the path of the bundle of His over the membranous septal floor it give rise to the right and left bundle branches.
      4. The Right Bundle originates from the branching bundle (during its path over the membranous septal floor) and travels along the right side of the interventricular septum toward the medial papillary muscle of the right ventricle
      5. The Left Bundle originates from the branching bundle (during its path over the membranous septal floor) and travels along the left side of the interventricular septum
d. According to the above methodology one can localize the landmarks that describe the location of the conduction system axis of a patient. These annotations can be achieved manually or by image processing of CT or MRI using the difference between the elements attenuation properties (HUE in CT) or T1 weights in MRI, or can be generated by a ML network or a combination of 2 or more of the techniques.

For example, for a HUE based method in CT, an image processing method performing HUE filtering of the Cardiac CT and generating CT images of values of HUE that corresponds with tissue like fat, fibrous, connective tissue may be performed. For example the filtering select a window of HUE of values below 30 HUE or below 0 HUE. In some embodiments, by generating multiple images of different HUE windowing. In some embodiments by using a dual beam CT image and selecting a different HUE for each beam depending of the beam energy. In some embodiments in a dual beam image using a ratio between the two energy values of the corresponding voxels. In some embodiments using a conventional cardiac CT and processing the image into multiple HUE windows and using a derived ratio formula that enhances the difference between the above mentioned tissues and the rest of the myocardium and the blood within it.

In some embodiments of the invention, the membranous septum and the membranous septal floor are identified by searching the most proximal endocardium of RV and LV area.

In some embodiments of the invention, the membranous septal floor is identified for example by drawing a highest elevation line of the myocardium and identifying the crossing of the membranous septum (e.g., previously identified) and the myocardium highest elevation line. Once identified, the Infrapyramidal space (IPS) and the inferoseptal recess (ISR) are optionally segmented.

In some embodiments of the invention, the following points are identified and/or marked:
1. AV node (Point A) sits at apex of IPS closest or at the intersection between IPS and ISR (in one embodiment).
2. Point B sits on intersection of ISR and posterior border of MSF (MS floor) in (in one embodiment)
3. The Non branching bundle of His extends from point A to Point B over the surface of the ISR.
4. Point C sits on the MSF at, for example, 70% of MSF length (other values, for example, between 50% and 80% may be used)
5. The Left bundle (sheath) starts emerging from the MSF and will commonly continue to extend all the way to 100% the MSF length and cover the endocardium of the LV on its common path a distance of, for example, between 1-4 times the MSF length and then the Left Bundle will break into fascicles and each will continue its path toward the anterior, posterior papillary muscles and the LV apex (superior, inferior and septal respectively).
6. The right bundle emerges as a narrow bundle at, for example, 60% of MSF (other values, for example, between 40% and 80% may be used)

In some embodiments of the invention, locations in the atrial conduction system are identified.
1. The SA node is optionally identified by identifying the sulcus terminalis on the epicardial surface of the right atrium (e.g., fat surrounding and separating the SVC and RAA) in a CT, typically is located lateral side of the SVC.
2. Bachmann's bundle is optionally identified by identifying points as follows:
   (a) BAC 1—Point on the SVC RA junction in a medial-posterior position. On the border between RA and RAA.
   (b) BAC 2—a point between the roof of the LUPV (Left Upper pulmonary vein) and the Left Atrial Appendage.

The following anatomical structures/landmarks may be useful for identifying the atrial conduction system and are generally suitable for automated identification on a structural image due to material and/or geometry:
1. Sulcus Terminalis: A fat-filled groove on the external surface of the right atrium. This can be identified by material and shape on a structural image. The Sulcus Terminalis typically corresponds to the crista terminalis and marks the boundary between the smooth and rough parts of the right atrium. The sulcus terminalis is located close to the superior cavoatrial junction. The SA node is located there as well.
2. Crista Terminalis: The crista terminalis is a prominent muscular ridge in the right atrium. This can be identified by material and shape on a structural image. The Crista Terminalis separates the smooth-walled Sinus Venosus from the rough pectinate muscles of the atrial appendage. Typically, the crista terminalis extends from the superior vena cava to the inferior vena cava and the pectinate muscles extend from it.
3. Fast and Slow Pathways: These are part of the atrioventricular (AV) nodal conduction system and/or are part of the inter-nodal conduction pathways. The fast pathway is typically located in the transitional zone leading into the compact node, while the slow pathway is an extension of the inferior nodal. The crista terminalis connects to the slow pathway and the interatrial septum connects to the fast pathway. In a healthy heart, the fast pathway is the main route for conduction from the SA node to the AV node. The slow pathway can act as a backup.

### Planning

While planning of a cardiac procedure can be started before providing conduction system data, it is a particular feature of some embodiments of the invention that planning takes into account such data. Optionally or alternatively, the execution of such a procedure uses conduction system data, for example, as will be described below.

A simplest type of planning (510) uses a visualization of the conduction system (507) for example for manual planning (e.g., a user marking possible locations related to activities and evaluating by eye).

A more sophisticated type of planning uses one or more of the following tools:
(a) combined visualization (507) showing structure, conduction system and more data, such as functional data or electrical data;
(b) simulation of effects of a proposed treatment (512);
(c) geometric planning of access and/or layout (514);
(d) automated or semi-automated search of a solution space for effects and/or geometry (516);
In some embodiments of the invention, a more in depth diagnosis is carried out after the conduction system data is available, for example, using ECG data (508).

During planning, visualizations and/or other data as expected to be seen during an actual procedure may be created and shown to a user. This may allow, for example, for a user to test-run a treatment.

After planning is completed, one or more outputs may be generated. In some embodiments of the invention, the output includes one or more of:
(a) views;
(b) synthetic images showing what should be seen at various stages;
(c) synthetic data showing what might be measured during a procedure;
(d) recommended fluoroscopic viewing angles and/or other settings;
(e) data for a computer for generating indications (e.g., alerts and/or a traffic-light type indication) showing a user if a procedure is progressing as planned; and
(f) instructions for a user and/or devices to be carried out at various stages of the procedure.

### Display

At 507 a display of the heart including the conduction system is optionally provided. In some embodiments of the invention, the display is a 3D display showing the conduction system overlaid or otherwise indicated on a 3D rendering of the heart. Optionally or additionally, the display is a display of one or more 2D projection images which can be used to simulate fluoroscopic views. Optionally, views not readily available in fluoroscopy, such as a transverse view, are generated and displayed.

In some embodiments of the invention, one or more of the following components of the conduction system are indicated. It is noted that the certainty of location (or spatial extent) may not be 100% and may be different for different components or parts thereof. Optionally, the display includes not only an indication of the component location but also an indication of uncertainty, for example, a lightly shaded area or dashed line. The following list shows one possible order of certainty of location:
a. Point B (penetrating bundle);
b. AV Node;
c. Point C (branching bundle);
d. LBB;
e. RBB;
f. Proximal Septal fascicle;
g. Proximal Superior fascicle;
h. Proximal posterior fascicle;
i. Distal Septal fascicle;
j. Distal Septal fascicle; and/or
k. Distal Superior fascicle.

In addition other data may be overlaid, for example:
(a) Targets (such a location on the septum to penetrate and/or a location in the septum to capture;
(b) places to be avoided;
(c) less or unviable locations;
(d) previously treated locations;
(e) suspected locations of blocks or previous ablation; and/or
(f) locations that are and/or were targets of structural intervention.

In some embodiments of the invention, instead of or in addition to marking conduction system locations, what is marked is anatomical locations. While such locations may be visible on a CT image, they are often not easily and/or precisely detectable on a fluoroscopy image. As will be described herein, marking can be used to indicate such locations during a procedure.

In some embodiments of the invention, instead of natural cardiac locations, what is marked are anatomically arbitrary locations which are otherwise selected for targeting. For example, when planning an ablation to block a reentrant loop one can identify the location on a CT image, possibly with the help previous electrical mapping. This location, however, may be in an otherwise anatomically arbitrary location on the wall of a cardiac chamber. However, the location can be identified with respect to gross anatomy. As is described herein, such location can be marked on a fluoroscopy image or other image generated during a procedure.

A particular type of arbitrary marking is guide marks for a procedure, such as locations to ablate or attach an implant or safety boxes (e.g., not to pass a line).

It is noted that the indication need not be on a cardiac structure. In one example, the indication is within a cardiac structure, for example, within a wall, such as the septum. The location may be shown with an error and/or certainty indicator and/or may be matched to a particular (or several) phases of breathing and/or cardiac cycle.

In another example, an indication may be in a lumen of the heart. Such indication may be useful, for example, to show a desired path of a catheter. In a particular example, such indication may be used to indicate a location of a radio-opaque marker or electrode or other identifiable part of a catheter or other interventional tool.

As can be seen, in general, any location which can be defined relative to cardiac structure can be indicated. In some embodiments of the invention, the definition is relative to cardiac structures in a particular phase of the heart, such as diastole. This phase may be standardized and/or may depend on the cardiac procedure. The phase may also be different for different cardiac locations. In some embodiments, however, an indication is located for multiple cardiac phases. When marked, for example as described herein, such marking can move with cardiac dynamics.

In some embodiments of the invention, when abnormal cardiac dynamics are suspected (e.g., due to an arrhythmia) a marking may be indicated as tentative. Optionally or additionally, different locations will be shown for different cardiac dynamics, for example, one location for regular sinus rhythm and another for when a heart is over paced.

An indication can also include what is expected to be measured (or seen) at a location during a procedure, for example, an electrical measurement or a physical movement. Such indication can be tied to, for example, a stage in the procedure, a triggering activity (which is optionally detected automatically), a manual request and/or a tool location. One way of identifying the viability of a location is used contrast CT and looking at the difference between uptake and washout in various areas-ischemic or scar tissue is expected to have slower uptake and slower wash-out. Another way is using electrical measurement data. Another way is from nuclear medicine imaging, such as PET or SPECT or other functional imaging modalities. In some embodiments of the invention, for example for pacing, scar tissue is identified manually or automatically as a location not likely to be captured by pacing, so it is not selected as a target area and/or its effects on signal propagation otherwise taken into account. It is noted that scar tissue may be a target area for penetration into a tissue in order to electrify viable tissue laying past it. Features other than scar tissue may be identified instead or as well, for example, one or more of myocardial viability, scar, fibrosis, amyloid deposition, contractile behavior, ischemic state, and/or inflammatory conditions.

In some embodiments of the invention, the display depends on the planned procedure. Optionally, the user enters the desired procedure into a user interface of the system.

For example, for performing conduction pathway interventions the following locations may be marked:
(a) Location of AV node (e.g., found within the fat of the infra-septal recess at the level close to the septal commissure of the mitral annulus.
(b) Location of the perforating bundle of the conduction pathway
(c) Location of the branching bundle of the conduction pathway
(d) The connecting path between points (a), (b) and (c)

In another example, for example for treating atrial and ventricular arrhythmia including for example reentrant atrial tachycardia (such as atrial flutter), atrial or ventricular fibrillation, one or more of the following locations can be marked:
(a) The circumference of the pulmonary veins in the left atrium;
(b) The isthmus in the right atrium;
(c) The endocardium and/or epicardium around a ventricular scar;
(d) The endocardium and/or epicardium around a ventricular area of myocardial compaction or other anatomical or functional (e.g., as seen with specific contrast enhanced imaging for example, higher or lower sympathetic activity); and/or
(e) The outline of a box procedure path on the left atrial wall or other procedure guide.

In another example, of performing atrial trans-septal puncture sites, one or more of the following can be indicated:

The RA puncture point, the LA point, and/or a line assuming one pre-planned optimal trajectory between two electrodes of a tool. For example, when using a sheath containing the needle with two radio-visible markings, have one location indication in a known position proximal to said sheath distal opening, with a second indication being a desired distal end location of the needle.

In addition to or alternatively to automatically added markings (e.g., based on preset or procedure) and semi-automatically (e.g., a user selects from a list or by name), a user can optionally modify the location of an added marking, for example, by editing a 3D location, for example by dragging on a 3D image. Optionally or additionally, a user can add a new marking, for example, by indicating a 3D location and selecting a marking type (e.g., dot, sphere, line or other geometric shape). Optionally, the marking system allows a user to snap a marking to a cardiac structure, such as a cardiac wall.

Different indications may have preset graphical properties when displayed and/or user selectable properties, for example, line shape, transparency, color, shadow, width, texture and/or variations of any of the above.

Other data can be shown as well and/or have indications added therefor in some embodiments. This includes, for example, electrical information and tissue viability and/or type.

Visualization may include a 3D image visualization. In some embodiments of the invention, 2D visualizations and, in particular, 2D projection visualizations are shown. One type of visualization shows an expected view during a procedure. A user optionally adds indications to be shown that are tied to particular visualizations. This may be useful as changes in visualizations directions may correlate with stages of a procedure, allowing data to be shown as needed.

In particular, it may be useful to show non-parallax visualizations, e.g., which have a point target on the left bundle and the right ventricle penetration point align. This can include selecting the LAO and CRA angles of the fluoroscopy such that they belong to the corresponding S curve. For example, this can include portrait and enface views of the heart. In some embodiments of the invention, an enface view is a non-parallax view of the left bundle and its fascicles viewed at the LV septum from a left lateral view where the dimensions of the left bundle and the fascicles are kept without parallax. In some embodiments of the invention, a portrait view refers to a view from the point of penetration of the septum that includes in the image the target point for pacing and the distance between the penetration to the target is kept accurate.

Another type of visualization is a virtual visualization not possible to be acquired during a procedure. A particular example is a transverse view of the heart. This may be useful to help a user see a trajectory and/or amount of penetration of a pacemaker lead tip from a right side of the heart to the left side of the heart to a target pacing location.

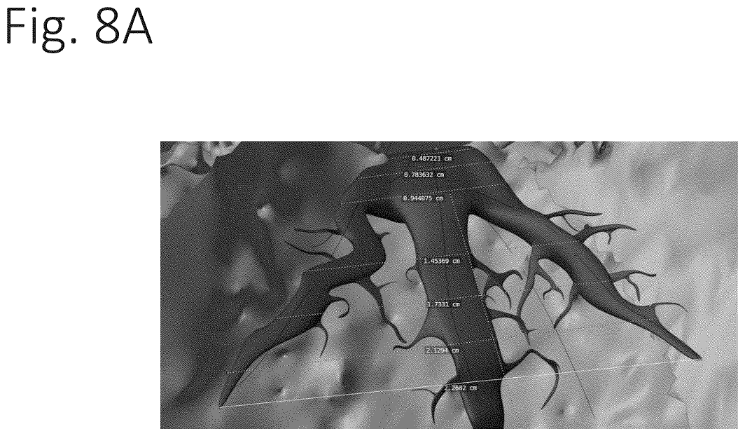

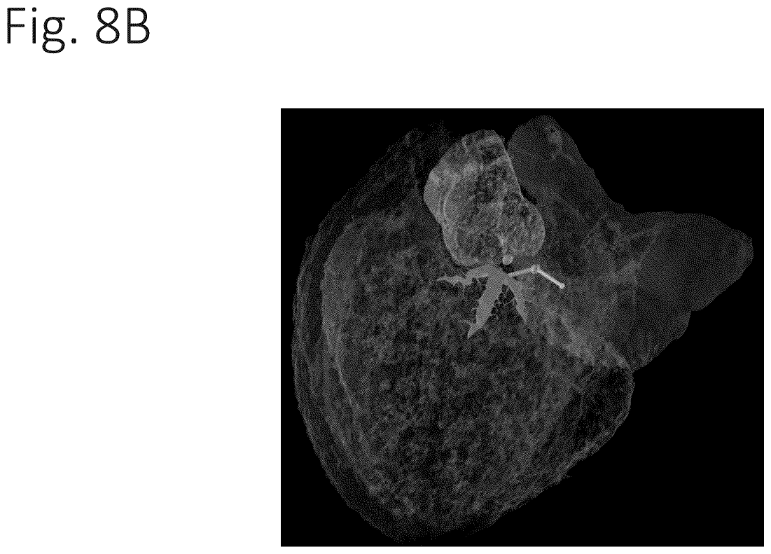

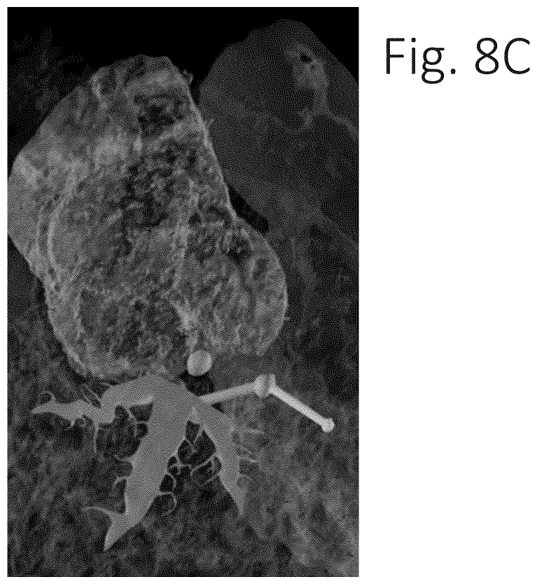

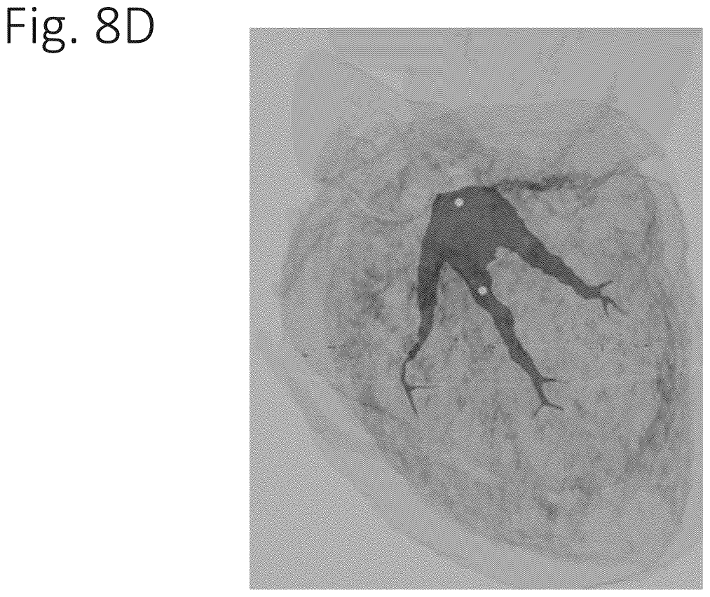

FIG. 8A shows an example template for a left bundle branch. During visualization, this template may be parameterized and overlaid on an image at a location of the left bundle block as determined, for example, by CT analysis.

FIG. 8B shows a 3D segmented view of a heart, for example, based on CT image data and on which the left bundle branch of FIG. 8A is overlaid. Also shown are the conduction pathway from the AV node to the LBB, including the bundle of His.

FIG. 8C is a zoomed in view of FIG. 8B showing the conduction system parts.

FIG. 8D is an enface view of the left ventricle showing the left bundle branch and its three fascicles, from right to left: LPF, LSF and LAF.

Some additional markings, not shown, one or more of which may be provided, especially for LBB-related treatments, include:
(a) continuation of the fascicles to the base of papillary muscles. One continuation is clockwise and the other is counterclockwise. In some embodiments of the invention, the continuation is truncated at, for example, 30%, 50%, 60%, 80% or intermediate or smaller percentages of the path connected to the papillary muscles
(b) a line connecting point C to the LV apex; and/or
(c) The right bundle, for example, shown as a path from point C to the medial papillary muscle of RV and/or a path or part thereof from the medial papillary muscle to the moderator band.

In some embodiments of the invention, marking is by taking templates (e.g., such as shown FIG. 8A), positioning them according to the conduction system location and morphing them to lie flat on the ventricular wall (or other anatomical structure). In some embodiments of the invention, such overlaying of a template includes scaling the template, for example, according to the LV dimensions, such as width at base and/or vertical height to apex. Optionally, a table is maintained mapping LV geometry/measurements to template deformation. In some embodiments of the invention, scaling includes setting the apical length of the LBB to be 5% of the distance between point C and the LV apex. Other values can be used as well, for example, between 3% and 10%.

### More Diagnosis

In some embodiments of the invention, further diagnosis is performed during the planning stage. In some embodiments analysis of the CT image can be used on its own or with other data to better understand the condition of the patient.

One example of further diagnosis involves combining electrical information with structural information. For example, ECG data can be used together with the conduction system data to better evaluate a patient situation and/or plan a more effective and/or safer procedure.

One type of diagnosis which may be useful is identifying potential locations of conduction system blocks using ECG.

For example, ECG analysis of, for example a 12 lead ECG, optionally in the form of a vectorcardiogram may be used to detect an infra or supra nodal conduction disturbance.

In some embodiments of the invention, an analysis of 12 lead ECG is used to create a vectorcardiogram by transforming the 12 lead data into data from co-planar composite leads. In some embodiments of the invention, the vectrocardiograms are analyzed to show the progress of the ventricular activation front over time. Such progress may be used to generate a rating for the well-being of some or all the entire Left Bundle and/or its fascicles. In some embodiments of the invention, the ranking is based on general measures, such as rate of change. Optionally alternatively or additionally, the progress is compared to normal progression rates as measured in healthy hearts or in hearts with particular pathologies. Optionally alternatively or additionally, a machine learning based classifier is used to classify the vector gram and/or its progress. Such a classifier may be trained on vector electrocardiograms collected from hearts with known pathologies.

In some embodiments of the invention, the LBB state is used to plan a location for pacing, for example manually e.g., by showing a projected state of the LBB and/or automatically, for example by calculating a blockage location in the LBB and suggesting a pacing location below such location.

In some embodiments of the invention, ECG analysis is used to identify deviations in the ECG of a patient as compared to a normal ECG, a past ECG in the patient and/or an expected ECG in a patient with such pathologies as the patient is known to have. Data generated by the analysis may be used in planning a treatment, for example, in planning a treatment which results in a smallest deviation of the resulting ECG form a target (e.g., normal) ECG.

In another example of further diagnosis, the ECG is analyzed to detect myocardial infraction locations. Such locations may be untreatable and/or may block conduction and/or may be a target of therapy. For example, methods such as discussed in the following article may be used: “Deep Learning for Detecting and Locating Myocardial Infarction by Electrocardiogram: A Literature Review” by P. Xiong, S. Ming-Yuen Lee & G. Chan, Front. Cardiovasc. Med., 25 Mar. 2022, Sec. Coronary Artery Disease Volume 9—2022 doi: 10.3389/fcvm.2022.860032.

In other examples of further diagnosis, non-ECG data may be used, optionally with ECG data. For example, location of scar tissue is used to evaluate expected ECG behavior and compared to actual measured ECG to identify potential problem parts or undiagnosed problems in the conduction system.

### Geometric Planning

Actual treatment of the heart typically includes two geometric-based constraint issues which may affect treatment or other access to the heart. In some embodiments of the invention, one or both of these issues are considered during planning. In general, geometric considerations can be relevant not only for treatment of the conduction system (e.g., pacing or ablation), but also for other procedures, such as “structural heart” such as implanting, replacing and/or repairing an implant such as a valve, as such interaction with the heart by the implant and/or delivery system hay cause damage to the conduction system, for example, by applying pressure or shear forces.

A first issue is access. The heart is typically accessed in one of two ways. In a first manner rigid tools are used and then care must generally be taken to avoid damaging critical structures. This limits possible access paths. In a second manner, catheters or other flexible tools are used and which can deform to follow the shapes of lumens in the heart (though some tissue penetration, such as the atrial septum, is often used). However, the geometry of the heart affects the directions from which a target can be accessed.

A second issue is success of treatment as affected by geometry. Depending on the access direction a procedure may be more or less likely to fail and/or otherwise be suboptimal. For example, penetration into tissue and/or correct coverage of a target for lead placement may depend on access direction. In another example, a pacemaker lead may be more likely to fail (or a more expensive or thicker lead needed) for certain access directions. It should be noted that combination of access direction and target location can define a bend in the catheter and/or expected movement during cardiac activity, one or both of which may have a degrading effect on such lead.

Typically, but not always, a first consideration is the location to where treatment is to be provided. A second consideration can be the location from which the tool is provided (and any limitations on tool flexibility and/or steerability. For CSP (conduction system pacing) or ablation in the right ventricle, the location from which the tool is provided is usually the tricuspid valve.

For example:
(a) for reaching the slow pathway of the AV node, the target for ablation can be more superior than the marking of the AV node (point A as marked on the right ventricular wall);
(b) for reaching the fast pathway of the AV node the target for ablation is more to the right and superior of point A;
(c) the target may be near the point A marking of the AV node that operator, based on operator selection based on a desired intervention;
(d) for reaching a point on the left bundle that is below the conduction block
   a. Based on the QRS axis of the ECG the operator may decide how far below point C is appropriate to deliver the pacing;
   b. To reach optimal hemodynamic response to conduction pacing the operator may try to activate the left bundle as high as possible in its path toward to the inferior segments of the left ventricle;
(e) to perform biventricular pacing using a single pacing lead the operator may choose a trajectory inside the septum where a distal ring electrode of the lead (the LV electrode) is positioned within the left bundle and/or a proximal point for activation (e.g., where a proximal ring will it) is positioned next to the right ventricle endocardium; Optionally the user/operator either notifies the system regarding inter-electrode spacing or the system will suggest (e.g., based on simulation as described herein) a suitable lead for such pacing;
(f) for some types of biventricular pacing, two leads will be implanted, each with its own geometric considerations and target; and/or
(g) to perform conduction system pacing achieving both activations of the left and the right bundle the operator implants in certain qualified patients, the target location may be the highest position on the path just below the branching bundle (optionally indicated as point C); this may provide for physiological pacing of both the left bundle and the right bundle.

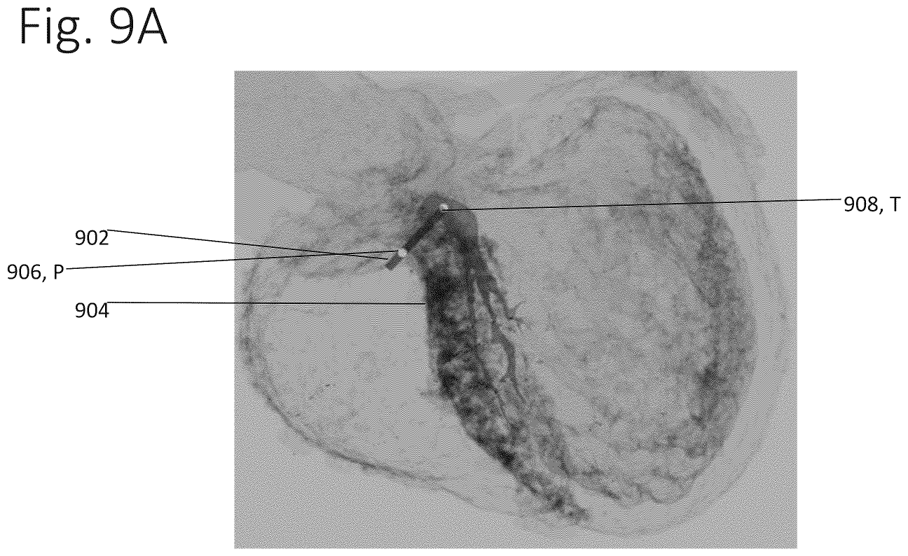

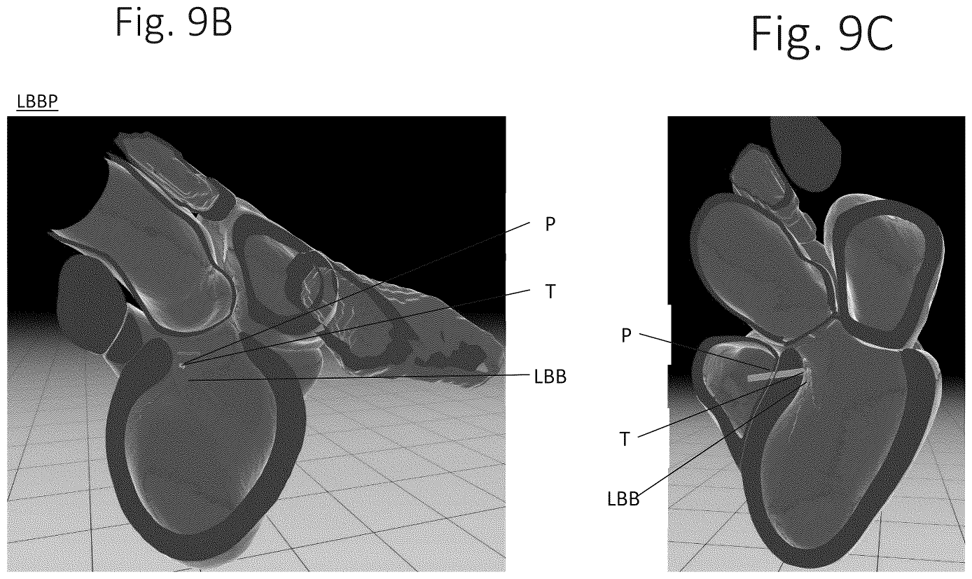

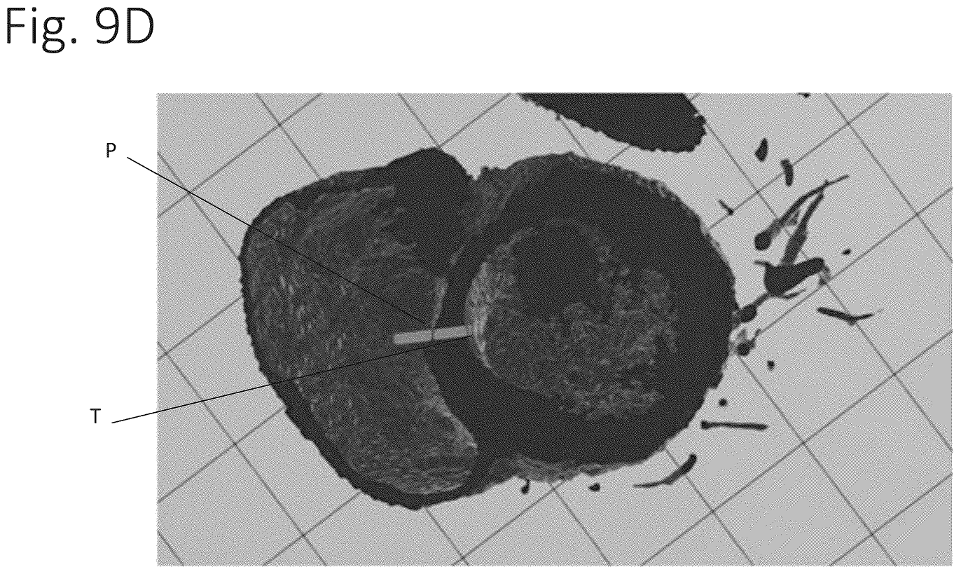

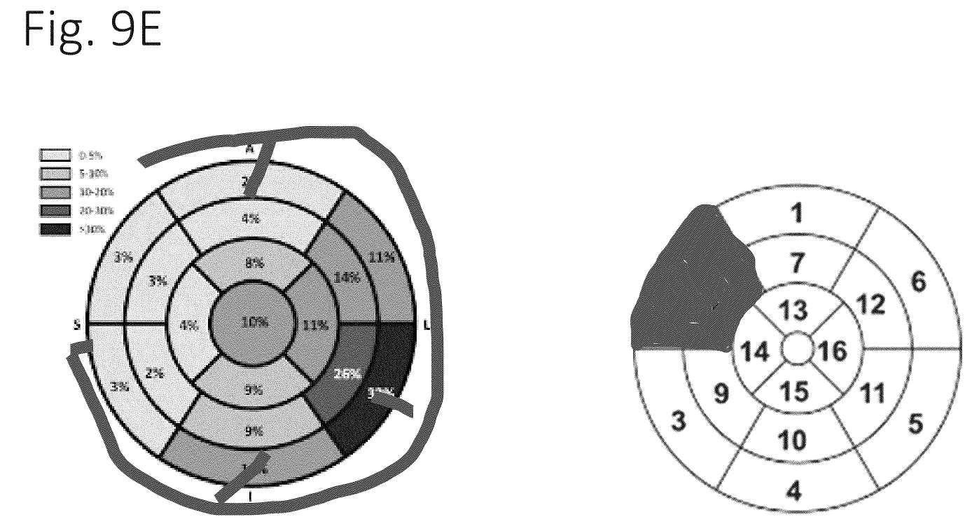

FIG. 9A shows an example visualization of trans-septal LBB pacing, the path is shown as a short cylinder 902 penetrating a septum 904 at a penetration (P) point 906 and continuing to a LBB capture point (T) 908 (which may correspond to a radiopaque marker; if not, a lead-tracking annotation may be provided during navigation, for example as described here). If carried out as planned, the lead might penetrate at point P and be screwed in (or an extension thereof advanced and/or screwed in) to reach capture point 908. It is noted that for each lead a there can be defined how many rotations of the lead result in what distance of penetration into a septum, for example, between 5 and 13 rotations. In some embodiments of the invention, part of the output of the planning activity is the number of rotations. It is noted that this number may depend on the actual thickness of septal tissue to be traversed, which may, for example, depend on the actual achieved point P. So even if the plan includes a recommended number of rotations, this number may change based on the actual penetration location. In some embodiments of the invention, the plan will include multiple such numbers, for example, for different leads (models) and/or for different penetration locations P.

In some embodiments of the invention, during planning and/or during actual implantation, the system may show the thickness of the septum, in addition to or instead of the thickness along the trajectory, at one or more points on the septum which may be used for penetration of a pacing lead. During a procedure, the pacing lead position and/or orientation may be used to report in real-time (e.g., frequent update such as faster than every 5, 3, 1, 0.5 seconds (or intermediate values and/or no faster than every 0.01 seconds) and/or on request and/or of the lead is moved), such distances.

FIGS. 9B and 9C are other views of a heart (e.g., using segmented CT data) showing a conduction system portion including the LBB (FIG. 9B) and a trans-septal cross view (FIG. 9C). FIG. 9D shows a transverse view.

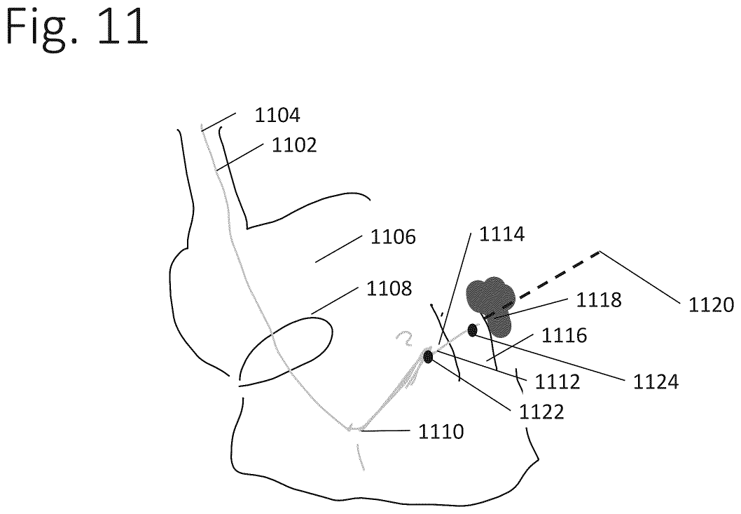

For example, in some implementations the following actions are taken by the operator and/or the system (some automatically or semi automatically by the system):
(a) loading a CT of the patient and calculating the conduction system layout;
(b) optionally shown the LV and its conduction system
(c) optionally show an enfase (non-parallax) view to the operator
(d) allow the operator to mark the target (T); this may also be done on a 3D image;
(e) optionally showing an enfase view from 180 degrees (e.g., from right ventricle);
(f) allowing the system to recommend point P (penetration); after which the operator can change point P; point T is optional fixed, though an operator can optionally change it;
(g) optionally the trans-septal path is shown on a view of the septal cross-section, for example as a dashed line;
(h) display of trans-septal distance and/or angle and/or suitable pacing lead;
(i) calculate two views and for each one report (and/or put in a plan) the LAO and/or CAUDAL of these views; optionally, once the patient is registered under fluoroscopy these values are corrected and reported to the operator:
   (1) Side view (FIG. 9C) shows T on the right, P on the left, both are place in a graphical dashed area extending straight 1 cm to the left of P—this is the landing path for the operator to make sure his sheath is correctly oriented and positioned (see also FIG. 11).
   (2) overlay view (FIG. 9B) where T, P and the trans-septal tunnel (connecting P and T) are positioned one above the other (it's a perpendicular view to View 1)—this is an overview of the landing path—the sheath/lead is just above P and the Tis straight below it.

As can be seen, one result of such process is viewing angles usable for fluoroscopy during a procedure and including optionally views where radio-opaque markers and computer generated markers are expected to be inline (for a side view) or overlapping (from a view along the path). Such views may be simpler to use in a procedure to ensure alignment.

In some embodiments of the invention, the location P is selected by the system to define a shorted path through the septum. Optionally alternatively or additionally, the location P is selected according to an inter-electrode distance between pacing electrodes on the lead to allow bi-ventricular pacing. Optionally alternatively or additionally, the location P is selected to allow better access based on sheath selection, availability or limitations. It is noted that the more superior points of the conduction system are typically more difficult to access for the RV for a catheter which enters from the tricuspid valve. Optionally alternatively or additionally, location P is changed in order to sit in scar tissue or to avoid scar tissue or other functioning or non-functioning cardiac tissue.

An additional possible consideration relates to ease of screw/helix/lead penetration. Some leads may have difficulty penetrating certain types of scar tissue and/or may sleep while being inserted. In some embodiments of the invention, the system takes such tissue properties into consideration when scoring a location as suitable for penetration.

An additional possible consideration relates to anchoring. For example, the free wall of an atria or a location covered with fat may be less suitable for anchoring a lead and/or may risk puncturing a hole in the heart. In some embodiments of the invention, the system takes such tissue properties into consideration when scoring a location as suitable for lead attachment and/or penetration.

Another possible consideration relates to sensing. Some parts of the conduction system may be sensed from the RV or RA. This applies, for example, to parts of the His bundle. However, other parts are covered with fat and may be difficult to sense in a non-penetrating way. In some embodiments, the planning includes selecting penetrating sensor. Optionally alternatively or additionally, ease of sensing form a surface is used when scoring a location as suitable for pacing, for example, if surface sensing may be used for conduction system verification.

FIG. 9E shows two charts. A first chart 950 shows degree of tissue viability and/or fibrosis. Such data can be obtained, for example, from CT imaging or from MRI or SPECT or PET imaging. Also noted is a schematic showing of the coronary sinus 952 and its main branches, potentially indicating possible locations for placement of a CRT electrode for the free wall of the left ventricle. A second chart 954 shows the same sectors and also showing where the main conduction system is located. In use, if the conduction system is traversed or covered by high fibrosis areas, it may be that conduction system pacing is less effective and rather CRT with an electrode in a free wall of the left ventricle should be used. Possible locations are the shown veins (which can be schematic or based on the CT data, for example. In some embodiments of the invention, fibrosis is such that also CRT might not be expected to be effective and/or that an epicardial electrode may be more suitable.

For simulations which search for a solution, it is noted that multiple trans-septal distances may be calculated, for different penetration points P. Similarly, for other procedures where penetration through tissue is needed, trans-tissue length may be provided in a similar manner.

In some embodiments of the invention, for example, for semi-automatic planning, the output of this stage is a plurality of suggested penetration points and paths for the trans-septal lead. For example, functionally similar target points and/or penetration points. Each such point may have associated risks, for example, of failure to deliver or of failure to provide the desired healing function (for example as explained here).

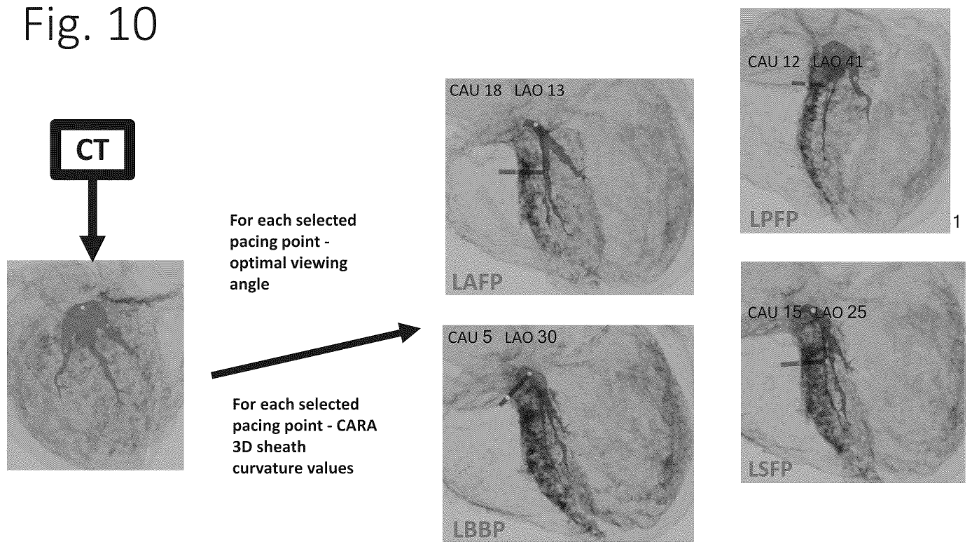

Referring now to FIG. 10 showing various trans-septal paths for different targets, in accordance with some embodiments of the invention.

The sub-image marked LBBP shows targeting of the left bundle. The sub-images marked LAFP, LPFP and LSFP show targeting of different fascicles. It is noted that the LAFP is shown lower, this can indicate a block lower down the conduction system than in the LPFP example. In any case, a pacemaker may be configured to time the stimulation in the paces Fascicle according to a sensed and/or calculated activation time in a parallel fascicle, for example, so as to provide balanced and more natural pacing of the heart.

It is noted that each pacing example is shown in a different view and with recommended angulations (CAU, LAO) of a fluoroscopy system during a procedure.

Geometrical considerations may also reflect limitations of sheath positioning and allowed pacing lead layout.

FIG. 11 shows a sheath 1102 arriving from a superior vena cava 1104, through a right atrium 1106 and a tricuspid valve 1108 and then curving 1110 inside the right ventricle before second-curving 1112 and extending along a straight path 1114 to a ventricular septum 1116 and penetrating (the sheath penetrates or a lead in the sheath penetrates) through septum 1116 to a target pacing location 1118 in the left ventricle or inside the septum, for example, a LBB location. While a pacing lead may be implanted in other geometries, FIG. 11 reflects a common geometry for pacing lead delivery, with the added feature of trans-septal penetration in accordance with some embodiments of the invention. Also shown in the figure is an extension of straight path 1114 past target pacing locations 1118, as a dashed line 1120. Also shown are two optional radio-opaque markers 1122 and 1124 on either side of straight path section 1114 of sheath 1102 (and/or on the lead). The first, proximal bend may be configured, for example, to reach an angle between 30-130 degrees, rotating posteriorly (e.g., range between +10 to −70 degrees) and the distal bending can be, for example over a range of −20 to +90 degrees).

In some embodiments of the invention, a “correct” sheath layout will include an alignment of straight path section 114 with a trans-septal path, for example as shown in FIG. 10. Moreover, the bending at curve locations 1110 and 1112 may be limited by the properties of sheath 1102. The user may notify the system which sheath or sets of sheaths may be used and/or their bending angles and/or adjustability.

In some embodiments of the invention, sheath 1102 is a sheath with fixed curves, for example part of a set and different curves are achieved using a different member of the set. In such a case, the planning system may try out various sheaths geometries to find a sheath which provides a “correct” sheath layout, e.g., as shown. Optionally alternatively or additionally, the curve values may be outputted and a user will select a closest match.

In some embodiments of the invention, for example, as described herein, a controllable sheath is used, for example a sheath with one or two or more controllable bending locations. The output of a sheath layout such as shown in FIG. 11 can be a set of instructions what angle to bend each sheath at. In some embodiments of the invention, the sheath layout is selected to allow the user enough leeway in bending the sheath (at one or both bending locations) during deployment to compensate for unexpected problems and/or shape mismatches.

In some embodiments of the invention, the search angle ranges are used as an input for a process of parametric searching (by the system) for a solution for sheath layout that also provides a desired path to target T and optionally also to point P.

Selection of a sheath path may also depend on the lead being used and which is to remain in the body after the sheath is retracted.

In some embodiments of the invention, interference of the lead with the operation of the tricuspid valve is used by the system to limit the path of the lead (and earlier, the sheath) to certain paths, which may set a constraint on how the lead can lie in the heart. It is noted that the lead is more flexible than the sheath and can bend differently from the sheath. In general, the path of the lead may be defined by the place where it passes through the tricuspid, the lead flexibility, and the contact point and contact angle (defined by the trans-septal pathway) with the penetration point. Various methods, for example, strain minimization modeling may be used to calculate the sheath layout and expected range of movement during heart beats.

In some embodiments of the invention, bending of the lead and possibly especially flexing of such bends during cardiac activity can cause failure of the lead. In some leads, other lead components may be at risk, for example, a forward extending helix used for penetrating the septal wall. In some embodiments of the invention, certain paths for the lead (and thus for the sheath) are forbidden or less preferred to reduce such fatigue. Fatigue is typically related to a maximum allowed bend radius of the lead and/or amount of flexing allowed over time.

In some embodiments of the invention, the lead selection will also be used to translate the intra-septal path into a number of sheath rotations to be used for septal wall penetration by a sheath forward electrode component such as a screw tip or a helix. In some embodiments of the invention, the planning system estimates a risk of slippage (e.g., due to angle), or other problems with lead penetration, for example due to scar tissue, which is optionally provided as part of the plan, e.g., for the operator (and/or the system used during the procedure) to consider and monitor while working.

In some embodiments of the invention, the planning system may try out several sheath, lead and penetration point options to identify one or more satisfactory combinations. In some cases, this may be done manually by a user and/or a user can place constraints, such as on locations, sheath type and/or lead type and/or restrictions on allows sheath and/or lead paths.

For some treatments, there is no long-term residing sheath, so sheath considerations may be ignored. A particular type of such treatment is a leadless pacemaker. While such pacemaker has no lead, the pacemaker itself may have a non-trivial size and geometric considerations considered during planning may include avoiding inadvertent contact between a body of the leadless pacemaker and the wall of the right ventricle other than at the penetration point, chordae, leaflets and/or another RV implant such as a lead or another leadless pacemaker. It is noted that in some embodiments of the invention a leadless device is used for CRT or other bi-ventricular pacing by being inserted in a way that one stimulation electrode reaches the LBB and the other reaches the RBB. This may require certain electrode (e.g., screw) lengths and/or electrode arrangement (e.g., two electrodes at a tip for bipolar stimulation of the LBB without stimulating the RBB).

In some embodiments of the invention, the output of such geometric planning can be a step by step description of the implantation process including when to bend a bend of the sheath and/or advance or retract a stylet. Such a description can include, for example, including crossing the tricuspid valve, while in the RV lumen, approaching the septum, approaching the puncture point within the septum (location and angulations) and penetration. Each step may include one or more of instructions (e.g., visual and/or audio), expected measurements, expected tip position and/or angulations, expected layout, a synthetic version of what an image (x-ray and/or ultrasound) acquired at that stage might look like, expected position (e.g., using a positioning system such as a magnetic or impedance positioning system) of some part of the tool or sheath, actions to take (such as contrast material injection), alerts and/or conditions at which such alerts will be presented. In some embodiments of the invention, no separate positioning system is used. Rather the acquired images and optionally other constraints are used for position-detection of tools (or for enhancing the accuracy of positioning systems).

It is noted that some procedures require more than one treatment location. For example, multi-point pacing may require multiple places to implant leads and ablation may include movement along a line. Such movements may interfere with cardiac structures, such as chordae. In some embodiments of the invention, such structures are taken into account when planning a path and/or a sequence of locations to which a sheath or other tool is to be breakout.

It is noted that such analysis of geometrical considerations as described herein can be used in other parts of the heart as well. However, there is particular potential benefit in the right ventricle, being a target for lead implantation, for the conduction system, where some parts are more difficult to access and/or for trans-septal penetration (such as to the LBB) where the path includes contact with tissue, floating in a chamber and fixed inside tissue. Other targets which share one or more of the properties may also potentially benefit from such planning and/or simulation as described herein.

### Treatment Simulation

Treatment planning may also depended on the expected outcome of the treatment. In some embodiments of the invention, planning includes estimating, for example by simulation, what such outcome might be. The results of such determining may be used to modify treatment parameters, for example, penetration or treatment location.

In the example of simulating pacing, a pacing location and pacing parameters may determine efficacy and/or other properties of pacing. In some embodiments of the invention, a FEM (finite element model) of conduction in the heart is used to model the heart, with each element representing a small part of the heart—e.g., muscle tissue, non-conducting tissue or the conduction system. Multiple tissue types may be provided, for example, for different muscle and/or for different parts of the conduction system and/or for various degrees of health.

In some embodiments of the invention, the model is personalized to the patient, for example, based on previous imaging or other collected data, such as data on electrical behavior, viability and/or ischemia.

In some embodiments of the invention, the model is verified, for example by comparing a virtual ECG generated by such heart to a real ECG of the patient. If needed, the model may be modified, for example by adjusting parameters (manually or automatically, for example by automated search), until the behavior of the model seems to match what is observed for the patient.

Other models of activity of the heart may be used, including, for example, an electromechanical model which also models mechanical behavior and an abstract model which models the heart as a graph. In some embodiments of the invention, only a part of the heart is modeled, for example, the left ventricle.

A basic simulation reports the effect of a certain treatment on the heart. This may be, for example, a manually triggered function used as part of planning. In some embodiments of the invention, a user may select one or more ranges of values of parameters and/or sets of such ranges and allow the simulation to map out the results over these different values. Optionally, such mapping is used to generate a data set showing various effects. Optionally or additionally, the mapping is applied as a search function to identify a satisfactory (or better) treatment. In some embodiments of the invention, a user may compare two or more treatments. In some embodiments of the invention, a user may generate a score for each parameter value set and optionally rank the value sets based on the score.

For example, one or more of the following parameters may be controlled in the simulation (such may also be varied during geometric simulation as described herein, for example):

Pacing location, pacing electrical parameters, pacing logic, electrode type, cardiac condition such as drugs, exercise status and/or hydration, ANS status (sympathetic and/or parasympathetic), breathing phase, preload, afterload and/or regurgitation.

The simulation can be used to show results of a treatment, e.g., an ECG, an activation profile of the heart, mechanical contraction behavior and/or other calculated patient physiological parameters, such as cardiac output.

In some embodiments of the invention, the simulation is used to generate statistical information which represents the results of more than one run and/or more than one set of parameters of treatment and/or underlying conditions.

In one example, the results show a distribution of success. For example, one set of parameters may result in a 90% success rate and another set may have an 80% success rate. Success may be defined, for example, based on statistics of the desired outcome, such as length of systole, shape of the ECG and/or how similar the resulting contraction is to a physiological contraction (e.g., in time and space).

In some embodiments of the invention, the result of the simulation is the type of failure and/or risk of failure. For example, a less risky pacing location may have a lower overall score, on the average, but be less likely to fail.

It is noted that the combination of potentially precise positioning of a lead and knowledge of the conduction system location potentially make such simulations and statistics more meaningful.

In some embodiments of the invention, a sensitivity evaluation is the result of simulation. For example, such evaluation may indicate to which variable the treatment is more sensitive.

In some embodiments of the invention, a source of likely failure is determined. For example, failure can be a result of doctor skill, accuracy of the reconstruction and/or patient variability. By trying out parameter variations to model each of these options, a degree of sensitivity and expected failure mode can be determined. This may help in selecting operators. In some embodiments of the invention, a better physician (e.g., with a better record) is selected if the expected failure mode is operator related. Optionally or additionally, depending on the quality of the available doctor, a different suggested treatment is proposed.

In some embodiments of the invention, the simulation is used to identify potential side effects and/or incorrect functioning of the therapy, as they relate to pacemaker (or other therapy) settings.

In some embodiments of the invention, such simulation is repeated and/or enhanced once the operation is in progresses and/or completed and more up-to-date data is available. Results of such simulations may include, for example, recommended pacemaker settings, expected pacemaker mis-operation and/or suggested changes in setting to handle such. Such results and analysis are optionally passed on to a handling physician in charge of programming the pacemaker after the operation and/or other care of the patient. In some embodiments of the invention, such information is stored on the pacemaker itself and/or linked in a database to an ID of the pacemaker and/or the patient.

In some embodiments of the invention, the simulation simulates a single beat or multiple single beats. In some embodiments of the invention, the simulation simulates a series of beats and models the effect of electrical conduction and/or contraction in one beat on following beats. One example is modeling the effect of hemodynamic effects on cardiac behavior. Another example is modeling cardiac activity through a breathing cycle and/or during an exercise ramp up or ramp down or steady state.

Another type of time related issue relates to the lead implantation. There is often a physiological reaction which can affect the outcome of treatment or at least modify it over a time period and/or which may suggest the need for different parameters (or expect different results) according to a timeline. This timeline may be measured in weeks after implantation.

For example, one or more of the following outcomes may be simulated: electromechanical effect of lead contact, edema and/or fibrotic cap.

In some embodiments of the invention, the outcome of such timeline is provided to a follow-up physician that treats the patient after the procedure. In some embodiments of the invention, the timelines and/or different pacing parameter sets for different times are provide din the pacemaker memory or in a database associated with the pacemaker ID. In some embodiments of the invention, pacing parameters change automatically under pacemaker control according to changes in timeline and/or according to one or more decision rule programmed therein which takes into account expected effects of a pacing regime and different sets for different conditions, such as different points along a timeline.

Following are some examples of results of treatment that may be simulated:
(a) finding positions for pacing electrodes and pacemaker settings (e.g., power) that can stimulate not only one conduction pathway portion, but several, for example two or more of LBB, RBB, atrium, and AV;
(b) finding satisfactory positions for bypassing a conduction block by pacing below the block (and possibly above the block);
(c) identify a pacing location where the effect of the applied field extends past a block;
(d) identify pacing locations for anti-arrhythmia pacing, arrhythmia counteracting (e.g., against reentrant VT) or prevention of arrhythmia. This simulation may benefit from a map of actual conduction problems and/or reentrant locations in the heart, which is optionally provided by EP mapping, such as using the Carto system by Biosense-Webster;
(e) identify pacing locations more stable under varying conditions of exercise and/or drugs;
(f) suggest one or more personalized pacing locations, optionally including locations that take into account damage caused to the heart by a previous pacing lead location;
(g) suggest locations for multi-point pacing, noting that precise localization helps make such pacing more practical; and
(h) simulation of the electromechanical result and/or hemodynamic result of pacing, for example, resynchronization, achieved for one or more pacing locations.

In some embodiments of the invention, a conduction system portion identified and localized is the location of the fasico-septal fibers (Mahaim) usually connecting between the non branching bundle toward the tip of the intraventricular septum close to the location of the branching bundle (point C). In some embodiments of the invention, simulation is used to assess the distance from the right ventricle endocardium and the Mahaim fibers. Pacing at this location may circumvent certain dysfunctions in the conduction system and such effects are optionally simulated.

Various models may be used to estimate the effect of a certain stimulation location and pacemaker settings on pacing effects. In particular the shape and/or intensity of the electric field generated by an electrode can be considered when deciding if tissue will be stimulated to generate a propagating action potential.

In particular, virtual electrodes models may be used, including for example, virtual anode-based capture estimation, optionally selected to relate to insulated (e.g., separated by collagen) conduction fibers, such as found in the LBB, rather than uninsulated fibers, such as the Purkinje fibers more distally along the conduction pathway. One of the pacing parameters which may be modified, for example by a user and/or as part of a search for a satisfactory outcome, is pulse shape parameters that affect the shape and/or other properties of the virtual electrodes.

In some embodiments of the invention, virtual anodes (or other pacing models) are used to determine if a certain pacing setting (including location) will capture the conduction system. Optionally, the simulation generates synthetic outputs, such as ECG and intra-cardiac measurement that match what is expected for various conditions of capture or non-capture. This potentially allows an operator to determine, during a procedure if the lead location and/or pacemaker settings are effective. In a particular example, a lead may be repositioned if not. Optionally alternatively or additionally, a helical tip may be advanced further or retracted according to such results.

In general, the pacing model used may take into account the directionality and/or other geometry of the pacing filed generated by the lead. In some cases, the system may evaluate several lead designs and recommend different settings for different designs and/or indicate one or more designs as satisfactory and/or better.

In some embodiments of the invention, the plan and/or annotations used during the procedure include volumes of regions expected to be affected by the virtual electrode, for example, indicating a range of locations expected to be affected by pacing, for example, indicating an error up to 2 sigma.

In some embodiments of the invention, the simulation takes into account anatomical considerations, for example, fat content, fiber direction, surface properties, and/or scar tissue. In some cases the anatomy is imaged. Optionally alternatively or additionally, the anatomy is estimated based on patient properties such as disease, age and/or fibrosis.

In some embodiments of the invention, a goal of treatment (or at least a measurement of success) is the normalization of surface ECG (and/or cardiovectorgram) post procedure. Normalization may be, for example, to the patients own baseline ECG (e.g., form the past or from a non-arrhythmic shape), normalization to a population ECG, normalization to ECG at other physiological states of the patient and/or normalization to a physician selected/input template.

In one example, a scoring function provides a normality score for an ECG and scores the outcome of one or more sets of pacing parameters (e.g., including location) and/or uses such score in a search (e.g., manual and/or automatic) process.

While the above simulation has focused on pacing and conduction system pacing in particular, simulation may be used to score the outcome and/or search for parameter sets for other treatments, such as ablation.

### Procedure

Referring back to FIG. 4 which shows a top level block diagram of a navigator sub-system which may be used during a cardiac procedure, in accordance with some embodiments of the invention.

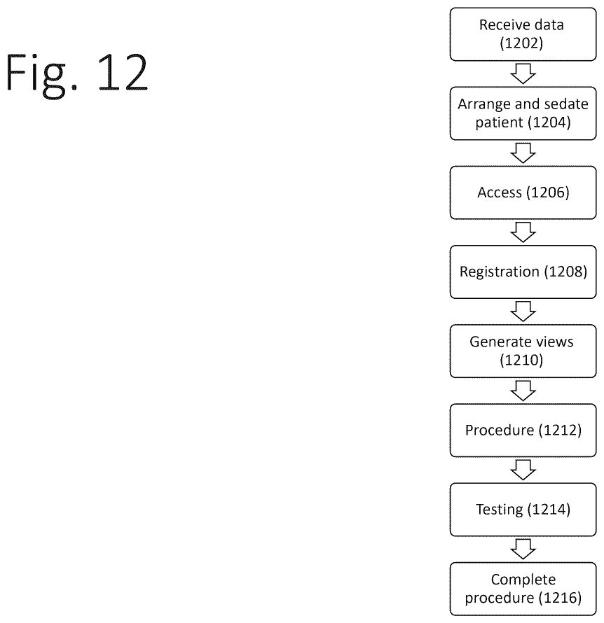

FIG. 12 is a flowchart of a method of an in-procedure diagnosing and/or treating a heart, optionally using the navigator of FIG. 4, in accordance with some embodiments of the invention.

At 1202 various data used during the procedure is optionally received, for example, a procedure plan, conduction system annotations and/or other data.

In some embodiments, what is received includes a pre procedure CT with annotations of anatomical sites that are important for the procedure and/or for image registration during the procedure, conduction system locations and data indicating a desired path and angulation(s) of tools during the procedure, for example, path and angulations at contact with a septum.

At 1204, a patient is arranged (e.g., on a bed in a cath-lab) and prepared for the procedure, for example, by sedation thereof. In some embodiments of the invention, the arrangement is selected to mimic the arrangement during acquisition of the CT image of the patient. If the patient is propped up, information about the propping may be used for a registration, to help align the CT image with a fluoroscopy image.

At 1206, the vascular system of the patient is accessed and interventional tools, such as a guidewire, catheter and/or sheath, are inserted.

At 1208, the patient is registered. This may include, for example, registration relative to a 3D positioning system. In some embodiments of the invention, no such separate position system is needed. Instead, registration matches an imager, such as a fluoroscope, to the pre-operational CT image (or other data).

In some embodiments of the invention, registration uses one or more of the following data types, which may be detected by analysis of one or more fluoroscopy images:
(a) Momentary C arm Caudal and LAO orientation, optionally starting at 30 degrees angle for each;
(b) Location and/or movement of reference catheters and/or interventional tools, for example, radio-opaque markers thereof and/or general silhouette;
(c) Location and/or movement of contrast enhance heart compartment; and
(d) Boundary of a heart lumen enhanced with contrast.

In some embodiments of the invention, reference catheters or other tools have a fixed location relative to an anatomical feature and can be used to locate and/or track that feature and/or assist in registration (e.g., as the tool should be matching the CT image anatomy). One example is a coronary sinus catheter placed in the coronary sinus of said patient.

As an example of tracking directly an anatomical feature, a contrast enhanced sequence of fluoroscopy images can be used to track movement of anatomy such as a septal (atrial and/or ventricle) wall, a valve annulus (e.g., tricuspid, pulmonary, aortic and/or mitral), for example based on leaflet motion.

In some embodiments of the invention, the actual registration comprises finding a best (or satisfactory) match between the pre-operative CT data and the currently collected information. Such a match optionally allows for tissue deformation and affine transformations.

In some embodiments of the invention, registration includes using data about physical contact. For example, if a physician indicates that a catheter is contacting a wall, and the catheter has a radio-opaque marker, then the location of this marker relative to anatomy may be known. In some embodiments of the invention, such indication may be used during the procedure to assist in determining a more exact position of a catheter or other tool relative to anatomy. An example of this is the physician indicating contact between a pacing lead and tissue with the tip of a lead, which tip has a radio-opaque electrode.

In some embodiments of the invention, the fluoroscopic image is segmented, for example, based on the 3D data set, after registration. Optionally alternatively or additionally, segmentation is before matching, for example, based on detection of chamber boundaries, anatomically linked tools and/or movements of one or both thereof.

In some embodiments of the invention, once the images are registered, the fluoroscopy image is analyzed to detect features which can be identified without contrast material and move with cardiac anatomy. Thereafter, these features are optionally used for overlaying anatomically-linked indications on a fluoroscopy image. In some embodiments, once the fluoroscopy image is registered to the CT image, it is assumed there is no patient movement (or movement is detected directly on the fluoroscopy image), so a same or adjusted registration may be used. It is noted that once registered, the fluoroscopy viewing angulations can be changed but the registration can be maintained, if the patient does not move or the movement is known.

In some embodiments of the invention, one feature which is registered (and possibly marked on the fluoroscopy image) is the AV ring. The AV ring may be important because it has a fixed relation to the inferoseptal recess and the conduction system. This has the potential advantage of assuring a higher accuracy of registration of important features. In some embodiments of the invention, one of the views used by a physician is an AV ring centric view (e.g., created synthetically, for example as described herein and/or generated by suggesting angulation angles for the fluoroscopy system) where movement of a catheter can be seen relative to the AV ring. For other procedures, other features may be chosen for registration.

In some embodiments of the invention, an anatomical feature which is shown on the fluoroscopy image as an annotation, possibly the only one, is the inter-ventricular septum.

At 1210, one or more views are created. One type of view is annotating a live (or frozen) image (e.g., fluoroscopy or ultrasound) with data. For example, conduction system layout (e.g., AV node position) and/or target areas and/or procedure plans or markers (including, for example, point markers, line markers and/or area markers) can be overlaid on such image. Optionally, such overlays include an indication of certainty, for example, shading, blurring and/or dashed areas being used to indicate uncertainty level.

A particular type of overlay, described in more detailed herein, is a marker intended to be aligned in a known manner with tool radio-opaque markers. For example, if the tool includes two radio-opaque markers, correct alignment with a target can be determined when, at a certain viewing angle, all three markers are aligned. Correct distance from target can optionally be determined from a view at 90 degrees, which shows the distance between the markers. Such determining may be manual. Optionally alternatively or additionally, such determining is automatic, by segmenting out the radio-opaque markers and checking their alignment with the virtual marker position.

Another type of overlay is a guidance, for example, a “corridor” for a sheath used during the procedure. This may be a preset (manually and/or automatically) corridor and deviations from this corridor are optionally indicated to the operator and/or generate an alert. In some embodiments of the invention, the corridor is used to suggest to an operator in which direction to advance the sheath. Optionally alternatively or additionally, the corridor is shown together with a suggested/preplanned path and/or target locations and/or trajectories. In some embodiments of the invention, the corridor indicates what the expected range of advancing of the sheath (or other tool) are.

It is noted that in a procedure tools often include radio-opaque markers and these are visible on the fluoroscopic image. In some embodiments of the invention, the marker locations are extracted and used for generating indications and/or indications are anchored to such markers. It is further noted that each tool may have its own markers. Optionally, in a kit, for example, a pacemaker kit, tools (e.g., sheath, lead and/or guidewire) have distinguishable markers, for example, based on shape and/or width, so the system can identify the tool based on its markers. Optionally alternatively or additionally, the tools and/or the kit have an ID, such as a QR-code or an RFID or other computer or human readable ID, which is associated with tool parameters such as location and/or shape and/or distance between radio-opaque markers. The data for such markers may be provide don the RFID or QR code instead of or in addition to an ID. Optionally or additionally, the operator selects a kit to be used (or the kit has such ID) and the system retrieves data matching such ID and optionally uses such data for registration, positioning and/or generating annotation s or for other calculations which use such data, for example as described herein.

Another particular type of overlay is an outline of the heart or parts thereof, such as the ventricular septum, possibly using dashed lines to indicate when it is an estimate. If a physician contacts a part of the heart and indicates this to the system such contact may be used to increase the reliability of the marking and it may, for example, turn solid. It is noted that even when all parts of a system are estimated at a high accuracy, direct registration between parts (e.g., the septum and the sheath), often allow a higher accuracy and/or certainty of positioning.

It is noted that registration and generating views are acts which may be repeated and/or adjusted throughout the procedure.

In one example, such adjusting relates to body movements, for example, such as caused by the cardiac cycle and breathing.

In some embodiments of the invention, annotations (e.g., markings in the overlay) are moved during the fluoroscopy to match their correct cycle and/or breathing dependent location by being based on a 4D CT that is segmented at multiple phases. Annotation and/or registration may then match the correct CT data segment to the physiological state at fluoroscopy acquisition (e.g., cardiac phase and/or respiratory phase. Optionally alternatively or additionally, breathing is measured and changes in the image which occur at a breathing frequency or harmonic are corrected for. Optionally alternatively or additionally, the effect of breathing on cardiac movement is modeled, for example, by extracting such effect from a fluoroscopy image and/or based on expected effect using data about a matching population.

In some embodiments of the invention, only a single phase cardiac CT is available and a prediction method is used to separate operator movements from physiological movements. For example, an ARMA (autoregressive moving average) model is created to classify changes in an image to Cardiac, Breathing, Operator induced and noise. The position of a lead (or anatomical marker) over a sequence of fixed phase fluoroscopy images is analyzed to show the Operator induced change only.

A particular type of view which may be created is a DRR-a digital reconstruction of a fluoroscopy image, based on CT data. This may be especially useful for showing views that are not possible, for example, a transverse view (the patient's head is blocking). A DRR view may also be useful for showing multiple live views simultaneously, when only one x-ray imager is available. Optionally alternatively or additionally, a 3D view is shown, using the data from the fluoroscopy image to annotate the CT data set with catheter (and/or other tool) positions.

At 1212 the procedure is carried out, for example, attaching a lead or a pacemaker to the heart, for conduction system pacing, noting that acts like creating views and registrations may continue throughout. Also, it is noted that the system may generate guidance, indication and/or alerts at any stage, for example, automatically or per request by the user.

In one example, for example depending on the stage of the procedure and/or tool location, the system may create and project (as an overlay), a theoretical direction in line with a pre-procedure planned orientation and/or a continuation of said tool direction.

In another example, data may be presented to the user, for example, on the fluoroscopic image or at a different display, concerning, for example, the suggested angulations for the C arm to achieve a navigation-assisting view.

In another example, the operator may be shown tool markets, targets and/or relative distance and/or alignments between them. In some embodiments of the invention, indications or alerts, such as alignment may be provided using a non-visual method, for example, as a sound.

In some embodiments of the invention, the stage of the procedure is automatically determined by the system, for example, based on the location of radio-opaque markers in the fluoroscopic image and their position relative to anatomical structures and/or each other. In some embodiments of the invention, one or more rules are defined that describe a state and/or a movement between states based on the contents of the fluoroscopic image. Optionally alternatively or additionally, an operator may notify the system, for example, using a GUI or by voice.

At 1214 testing may be made to see if the procedure was performed correctly (and changes made if needed. It is noted that testing may also be performed during the procedure, for example, testing a pacing location before lead attachment. One type of testing includes comparing an outcome of the treatment, such as pacing, to outcome predicted and/or desired during planning and/or stimulation.

In some embodiments of the invention, testing and/or prediction values are shown as an overlay during the procedure. For example, as a lead is used to scan pacing locations, the display may show an expected quality of pacing at that location. Optionally or additionally, an overlay indicating relative quality of locations (e.g., a heatmap) may be shown, for example, based on simulation.

At 1216, the procedure may be completed. In some embodiments of the invention, data collected during the procedure is provided to a further treating physician. In some embodiments, the data is provided in a memory of or associated with the pacemaker. For example, the pacemaker may be preprogrammed with recommend parameter sets or ranges which are expected to be effective and/or avoid side effects.

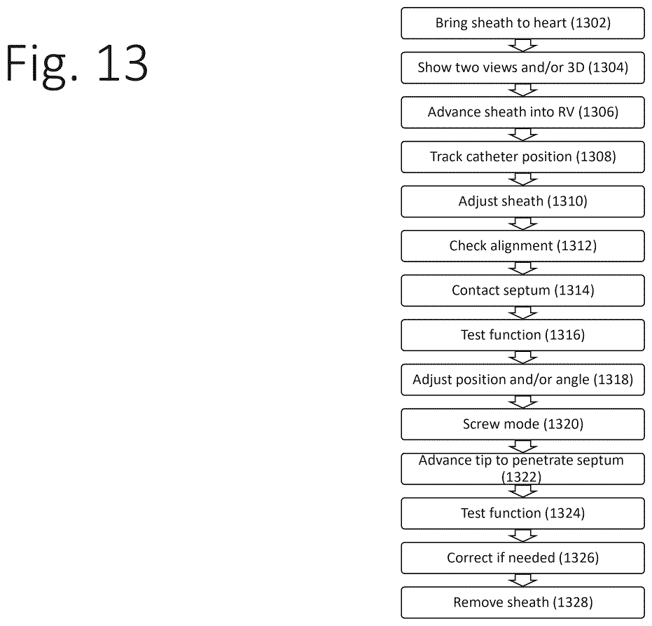

FIG. 13 is a flowchart of a method of lead placement for conduction system pacing, in accordance with some embodiments of the invention.

At 1302, the sheath (e.g., 414) is brought to the heart, for example, using conventional means.

The sheath may be a standard sheath or a non-standard one, for example, as described herein. In some embodiments of the invention, the sheath and/or pacing lead have an associated ID, for example, as a QR code or RFID code which is read by the system (e.g., using a reader, not shown) to know various parameters about the tool, for example, inter-electrode distance and/or flexibility parameters.

At 1304, appropriate view(s) are displayed. It is noted that these views may be provided sooner in the procedure as well.

In some embodiments of the invention, the view includes a synthetic 3D view, for example, as described herein. In some embodiments of the invention, the view includes two views. Optionally, one view is real (captured by an x-ray c-arm, for example) and the other view is synthetic, for example as described herein, for example a transverse view. In some embodiments of the invention, the two views are perpendicular to each other.

All these view types can be annotated. For conduction system pacing, for example, the conduction system is shown, for example, as described herein.

Optionally alternatively or additionally, annotations include relevant anatomical indications. For pacing, this may be the inter-ventricular septum, a penetration point, a target location (e.g., in LBB) and path therethroughbetween, optionally with distance indications.

Annotations may include stage specific annotations, for example, a recommend sheath path, which may include a suggestion on path position relative to the tricuspid valve.

At 1306, the sheath is optionally advanced from the RA/SVC junction towards and through the tricuspid valve and into the RV.

At 1308, the location of the sheath is optionally tracked, for example, manually. Optionally or additionally, the tracking is automatic, for example by comparing the radio-opaque markers of the sheath (or an internal catheter) to expected positions of such markers. Optionally or additionally, a positioning system may be used.

The views used by the operator at this point may be selected to assist in navigation. For example, a view may be selected which is perpendicular to a desired path of the sheath. As the sheath may include a bend and/or is bendible, different views may be used at different times.

At 1310, the sheath is optionally adjusted, for example, to follow the suggested path, to reach a desired penetration point P, to have a desired angle to the septum which will reach target T and/or to avoid chordae.

In some embodiments of the invention, adjustment comprises axial movement of the sheath and/or changing a bending angle at one or more joints of the sheath.

At 1312, the alignment of the sheath with a desired path is optionally determined. In some embodiments of the invention, this is determined manually. In some embodiments of the invention, the location of radio-opaque markers together the known registration of the CT data to the fluoroscopic image allow such determination to be performed manually.

In some embodiments of the invention, alignment determination is assisted by using appropriate views. This may be helped by activities in a planning stage or during the procedure. In one example, during planning An S-curve showing X Ray tube orientations that will create a non-parallax images of one or more of the conduction axis, sheath or lead and/or both is created. This indicates what images to use. If the procedure does not exactly follow the plan and/or as the tools move, new viewing directions may be determined, to avoid parallax. Optionally, two views are used, to avoid ambiguity possibly present with one view.

Such viewing angles are optionally presented to the operator at one or more times during the procedure, optionally triggered by sheath location and/or determined angulations (e.g., based on foreshortening of radio-opaque marker inter-distance).

In some embodiments of the invention, the suggestion of a non-parallax view takes into account information about the lead's (or sheath) mechanics and flexibility between the proximal and distal lead. In some embodiments of the invention, such view is based on real time detection of each lead principal component (e.g., radio-opaque markers, general outline) and checking its contributor from being in a non-parallax position, and decipher its real position. This may also be used when generating a synthetic view, for example, as described herein.

At 1314, the sheath is advanced to contact the septum. Optionally or additionally, the lead is advanced out of the sheath to contact the septum. In some embodiments of the invention, it is desired that the contact be at point P, the planned penetration point. Optionally, the operator indicates to the system that contact is made, which means that the position of the tip of the catheter can be mathematically constrained to be at a surface of the heart wall, for example, the ventricular septum.

At 1316, in some embodiments of the invention, the functional location is tested, for example, by sending an electrical signal through the lead and determining if the resulting ECG is what is expected.

At 1318, if needed, in some embodiments of the invention, the position of the tip is adjusted, for example, by modifying one or more angles of joints of the sheath (if they exist), for example, the last joint/bending region or by other manipulations.

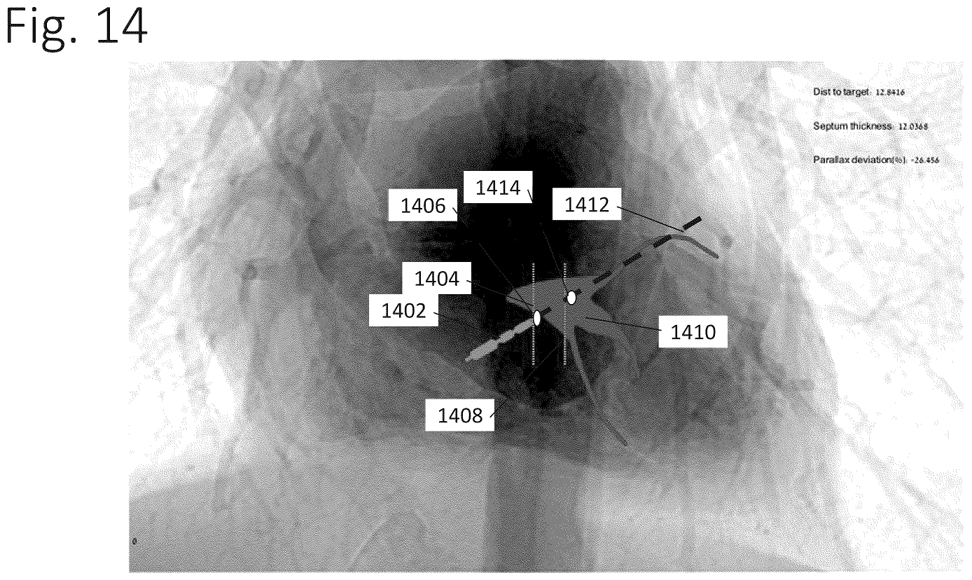

FIG. 14 is a side view showing a pacing lead 1402 in a common plane with desired penetration points (1406) and pacing points (1414) on opposite sides of a ventricular septum (marked with annotations 1404, 1408), in accordance with some embodiments of the invention. A vector 1412 created by the electrodes on a lead and/or radio-opaque markers on a sheath may be shown as well. In some embodiments, it is desired that the vector align with the path between the penetration and target points. An LBB annotation 1410 is also shown. If the image is real, rather than synthetic, lead 1402 will be visible as such, instead of or in addition to the annotation shown in FIG. 14.

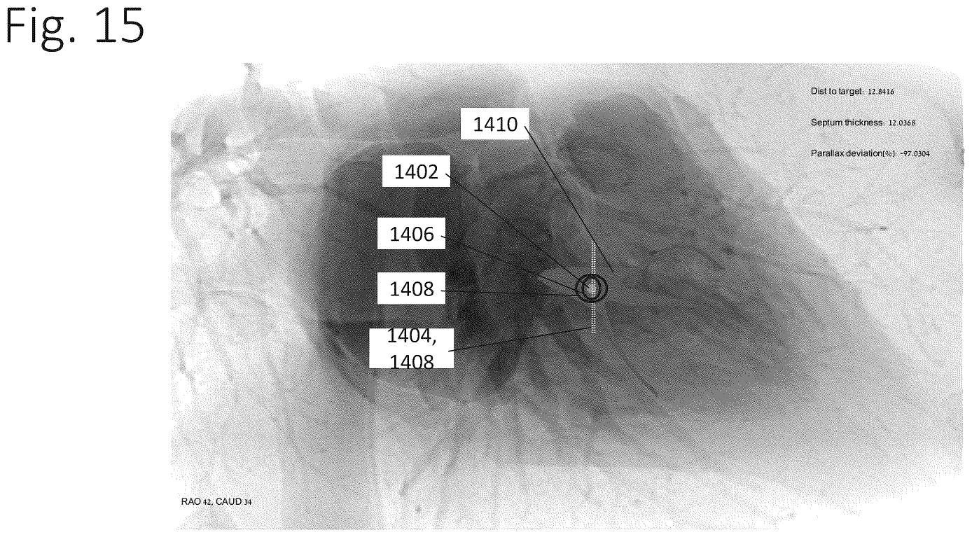

FIG. 15 is an axial view showing a view along an axis of a pacing lead and a line connecting a penetration points and a target pacing point on opposite sides of a ventricular septum, in accordance with some embodiments of the invention. As can be seen, if correctly oriented, only the markers 1402 for the lead is visible. Optionally, some annotations, such as 1406 and/or 1414 are shown as circles larger than the lead annotation 1402, so alignment with the target and/or trajectory can be seen as well.

These two views which may be shown before act 1320 or after can allow the operator to evaluate the alignment of the sheath/lead direction with the desired path through the ventricle to a pacing target T.

In some embodiments of the invention, the view (angulations suitable for) FIG. 15 (for the catheter and/or for the trans-septal line) is called a perpendicular navigateble view. In some embodiments of the invention, it is desired to match the anatomical needs (e.g., and so calculated to align with the trans-septal line). Optionally or additionally, it is designed to align with the probe. In either case, it is noted that when the two tool (e.g., sheath) markers are aligned on a line perpendicular to the view, only one maker is visible, if the two markers are the same size and shape. If one marker is larger then one might be seen surrounding the other. In some embodiments of the invention, the angulations for these views are calculated during a planning stage and/or recalculated if a physician changes a target location and/or penetration point.

When considering a fluoroscopic image, parallax can be detected by detecting foreshortening. For example, comparing a momentary distance between two lead markers to actual screen lead markers generates a measure of foreshortening. New C-arm angulations can be calculated so there is no foreshortening. Further, a C-arm angle can be further calculated so that there is planar alignment between the line connecting the two markers and a line indicating a desired trajectory (f any) or a point on the heart. The resulting view may be palled parallel navigable view, for example, as shown in FIG. 14.

In some embodiments of the invention, parallax indicators are generated for fluoroscopy images, for example, being shown over most or all of the procedure, for example, when the lead and/or sheath is in the heart.

In one example of using such views, an operator implants a branching bundle pacing lead to achieve correct anatomical positioning of commonly termed left bundle pacing. Using the planning process, an operator can select the appropriate NAVIGATBLE view they wish to use. In procedure, the C arm si moved to the calculated angulation generating such view and the tool (e.g., sheath) is maneuvered such that the markers match the selected view. For example, the markers can overlap in the PERPENDICULAR NAVIGATEBLE view and/or the 2D projection distance between the two markers matches the physical distance between the markers in the PARALLEL NAVIGATEBLE view.

In more detail, in an example of a LBP (left bundle pacing) procedure a peelable sheath is inserted from the Subclavian vein, going through the SVC into the RA, crosses the RV with pre-shaped or optionally personally adjusted curvatures—to reach the septal side of the intra ventricular septum-across from the LB.

In a planning phase points A, B and C are marked on the conduction axis. Optionally, the estimated location of the LB is derived by looking at the line connecting point C to the LV axis and drawing a theoretical LB triangle (the top corner is C and the middle of the base is at a certain length along the axis, for example 2 or 3 cm. A location within the triangle marked area is optionally marked on the LB estimated path, for example a certain distance such as 1 cm below point C toward the axis.

When navigating, the operator moves the sheath or lead so that the trajectory of the two markers on the distal end of the delivery sheath (which can be, for example, markers on the pacing lead, e.g., ring electrode and tip electrode with a known distance d) are in line with the pre-planned trajectory that crossed the septum from the RV-point of contact of the delivery sheath to reach the LV lumen. In some embodiments of the invention, the pre-planned trajectory includes a target point I located on the LV endocardium and within the LB triangle, and a second point on the trajectory is located on the continuation of said trajectory, for example at a distance of about 3 times the distance between ring electrode to tip electrode of the lead.

In some embodiments of the invention, the view is selected so that the septum lies in a plane. Optionally, this view (or other anatomically oriented views) are calculated based on the actual layout of the heart, for example as shown by a contrast injection. Optionally alternatively or additionally, the view is pre-calculated and an operator optionally makes a small change in angle as needed. Optionally, the c-arm reports its actual angle to the system.

A second view is optionally selected so that the septum lies perpendicular to the viewing direction. This may be useful for judging screw-in depth. In some embodiments of the invention, the views are selected according to desired sheath path, rather than anatomically, so one view has the sheath in a plane and one view is along a sheath axis.

In some embodiments of the invention, if a user does not aim the c-arm to a correct angle the correct alignment of markers is shown as non-point indications, such as circles or ellipses. Optionally or additionally, to such visual indication, a visual and/or audible alert may be provided if the user does not reach alignment and/or is off the desired path. Such alerts may be triggered, for example, if a user indicates that they are ready to move to “screw mode”. Optionally alternatively or additionally, the system may provide a user with advice on what to do, for example, how to move the sheath. Such advice may be generated in real time and/or be available (e.g., manually and/or automatically generated) from the planning stage.

Acts 1316 and/or 1318 may be repeated as desired.

At 1320, a screw” mode is optionally started whereat a helical tip of the lead is screwed into the ventricular septum to a point where it will be suitable for stimulating cardiac tissue, such as conduction system tissue not at the surface of the right ventricle. In general, the number of rotations indicates a depth of penetration. A desired depth of penetration may be provided as a result of the planning stage.

In some embodiments of the invention, during screw mode and/or at other times, two distances are indicated and optionally noted with length values (e.g., in mm). This may increase a confidence of a user in the system. For example, during screw mode, two distances may be shown to an operator-distance along the trajectory to target location and distance to closest far wall of the septum. This may assist in reducing concerns regarding exiting the septum into the left ventricle, with the helix/screw.

In some embodiments of the invention, during screw mode a transverse DRR view is used. The location of electrodes on a DRR view may be only estimated before the lead contacts the septum, but after contact, the position may be more definite.

At 1322, in some embodiments of the invention, the lead is rotated to screw a helical tip thereof into the septum.

At 1324, the functionality of the helical electrode is optionally tested, for example, by sending a pacing pulse. Optionally or additionally, a range of different pulses is used to assess the stability and sensitivity of the treatment. The range may be automatically generated by the system with each pulse parameter set being scored, for example, according to the ECG generated therefrom and/or compared to desired results described in the planning stage and/or entered by a physician during the procedure.

In some embodiments of the invention, testing comprises performing one or more EP measurements in the heart.

It is noted that while testing may involve verifying that the lead is located at, for example, the bundle of HIS, in some embodiments, the bundle is not found, or primarily found by HIS bundle mapping or other EP mapping, but rather using conduction system data as described herein. In some embodiments, such conduction system data is used to perform a relative limited (in space, based on the CS data) His bundle mapping process.

In some embodiments of the invention, testing is performed using a temporary pacing wire extended from the sheath and only after verification of the location and trajectory is a lead permanently attached.

In this and other embodiments and/or for this and/or other uses, sensing circuitry (e.g., amplifiers) may be integrated into the system (e.g., for control and/or data transfer and/or timing). In some embodiments of the invention, the system does not include sensing. If desired, a separate sensing system may be attached to the lead and used for measurement. Data from such measurement is optionally entered by hand into the system.

In some embodiments of the invention, tip and/or ring electrograms are measured repeatedly, for example, continuously and/or periodically and optionally collected to generate an electro-anatomical map (the structure known, for example, from the CT data set).

In some embodiments of the invention, some types of sensing and/or validation are performed while pacing or over pacing the heart, for example, by an external pacemaker and/or separate pacing wire.

At 1326, the position of the helix (or of the lead, by retraction) may be corrected, and appropriate acts repeated, if desired.

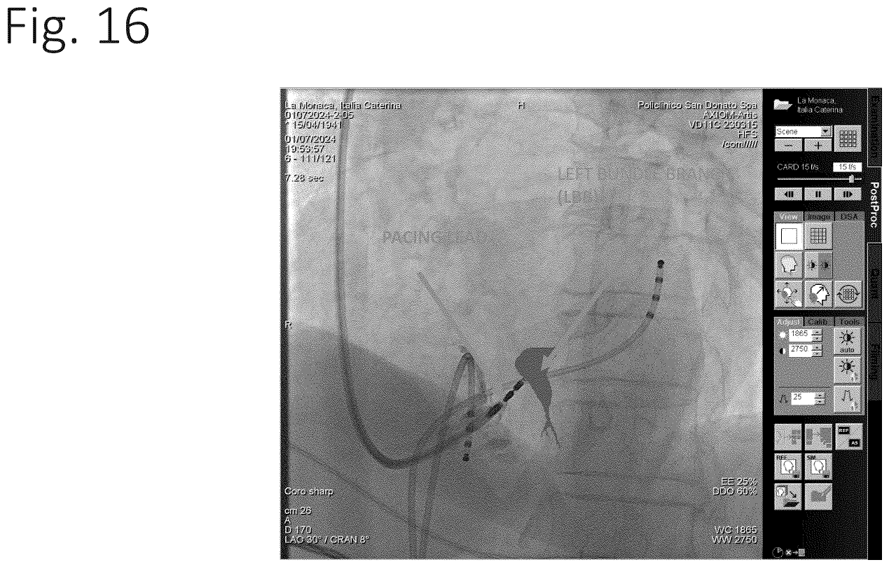

FIG. 16 is a fluoroscopic image showing a lead with a helix extending to an annotation representing the LBB, showing an exemplary pacemaker lead placement according to some embodiments of the invention.

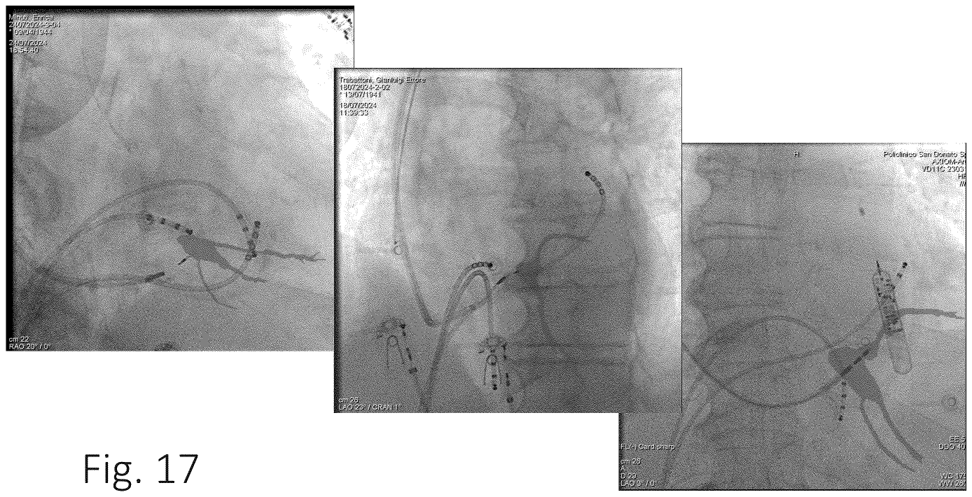

FIG. 17 shows several views of a pacemaker lead positioned relative to an LBB, in accordance with some embodiments of the invention.

At 1328, the procedure is complete and the sheath may be removed. It is noted that such procedure can be the same for implantation of a leadless sensor or pacemaker. The delivery system of such pacemaker is optionally modified and/or uses a sheath as described herein.

### Synthetic Imaging and Bi-Directional Mapping

It is noted that a transverse view is generally not physically possible but may be helpful in some situations, such as penetrating through a septum. The 3D CT data can be converted to a synthetic 2D projection. However, the image is more useful if it shown the sheath and/or other interventional tools. In some embodiments of the invention, the sheath position is identified in real time, for example, better than 1 or 5 or 10 frames per second, (using a positioning system and/or using image processing methods, for example, as described herein) and this is used to generate a synthetic view including the sheath and/or other tools. In some embodiments of the invention, the generation of sheath location is by matching. For example, different possible layouts of the sheath are tried out until a best or satisfactory match to what the fluoroscopic image shows, is found. In some embodiments of the invention, a suggested angle for the fluoroscopy imager is generated by the system to provide satisfactory or better data for such determining of sheath position and/or layout.

In some embodiments of the invention, a two directional mapping method is provided in that 3D-aligned data, such as data aligned with a CT image, is shown on a 2D image, such as a fluoroscopic image and data extracted from the fluoroscopic image is mapped back to the 3D alignment. Once this data is mapped back, the data may be shown in 3D, or a new, synthetic, 2D image may be created from the 3D data. This results in a 2D image, for example, a DRR which can simulate a fluoroscopic image, and which shows both annotations relating to the 3D data set and annotations relating to the 2D data, for example, live annotations. In some embodiments synthetic views are shown side by side with real views.

In some embodiments of the invention, the mapping back from the 2D data set to the 3D data set uses one or more constraints to restrict the possible mappings. In one example, a 3D orientation and/or shape of an object to be mapped back (or possibilities thereof) are deduced from the 2D image. Optionally or additionally, a physical interaction between the object and a structure is identified and sets a constraint on the possible mappings.

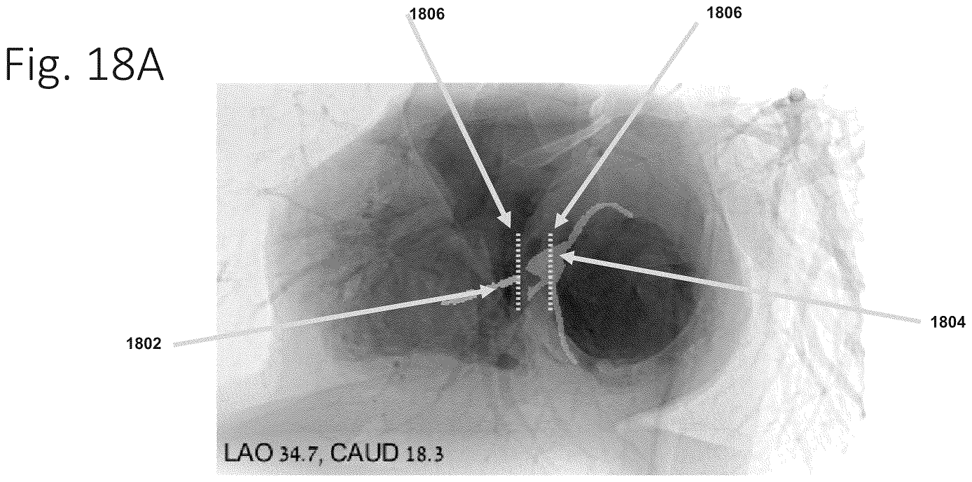

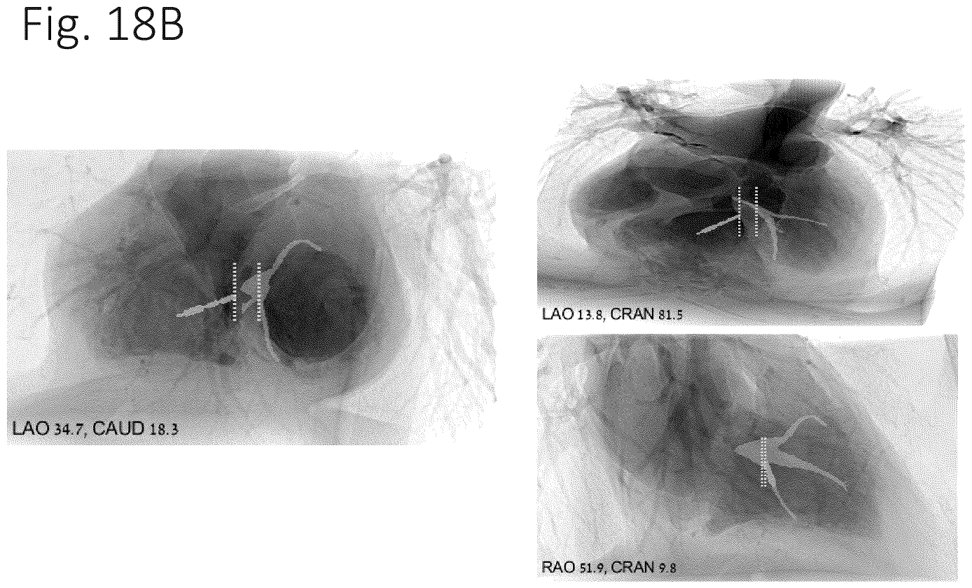

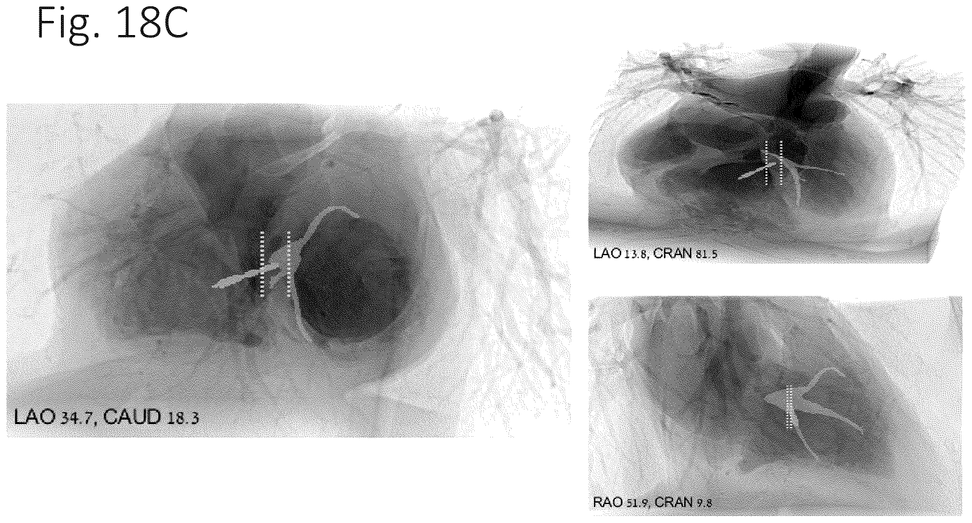

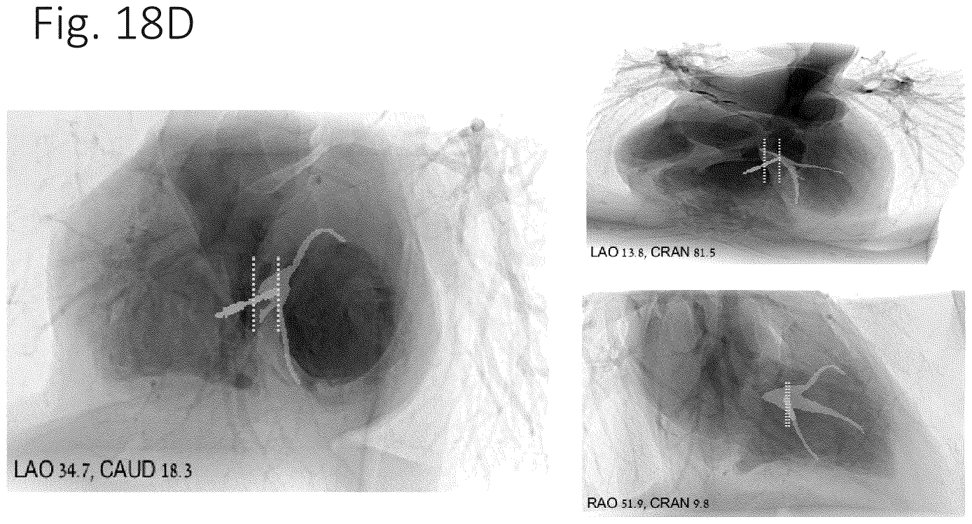

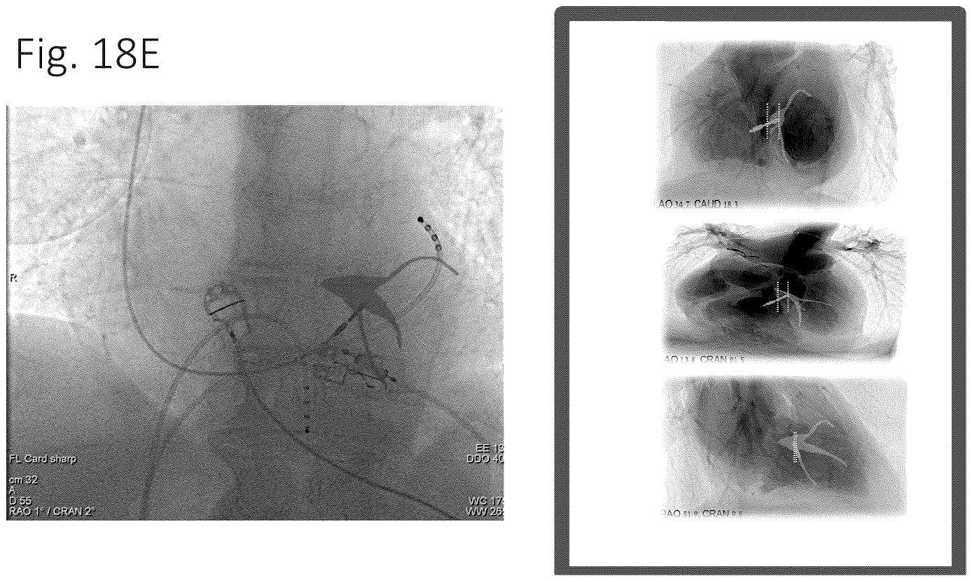

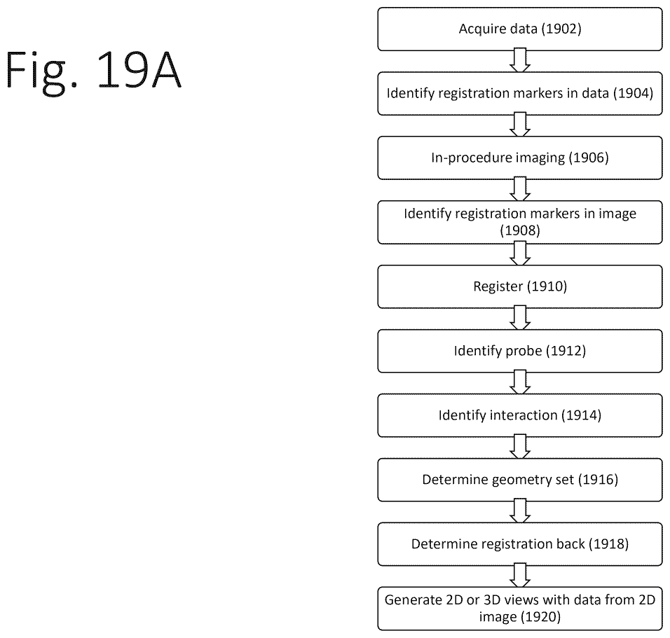

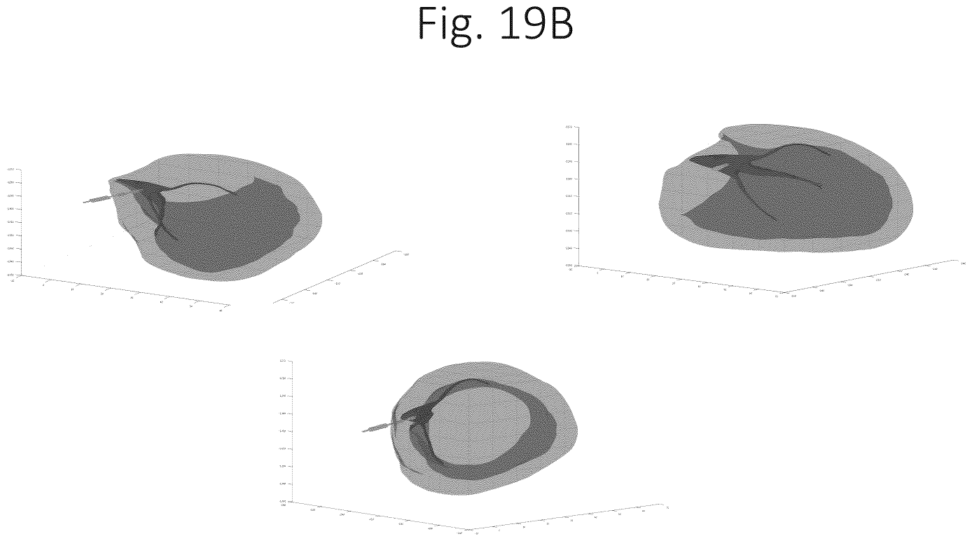

FIGS. 18A-18E and 19B show various views using synthetic imaging in accordance with some embodiments of the invention and will be described in greater detail below.

FIG. 19A is a flowchart of a method of generating a synthetic image including a mapping forward and a mapping backwards, in accordance with some embodiments of the invention.

At 1902 3D data is acquired, for example, a pre-procedure CT image, for example as described herein. The data is optionally converted into an image and/or processed manually and/or automatically to create annotations, such as a conduction system.

At 1904, one or more markers are identified in the data. One type of marker which may be identified is image registration markers, e.g., used for registering to a 2D fluoroscopic image. Such markers may include, for example, vertebrae, calcification, and/or vascular markers.

Another type of marker which may be identified is tool markers which are expected to interact with a tool to be mapped back, for example, the location of the right ventricular septum wall.

At 1906, during a procedure, one or more 2D images are acquired, for example fluoroscopic monoplane (or biplane) imagers may be captured continuously using a video grabber. It is noted that while fluoroscopic images are 2D (monoplane) images they actually are a projection image. Ultrasound images, another type of 2D image which may be used for this method are generally slice-like—an image of a 2D slice of tissue. A series of images may be useful for generating more registration information, such as due to motion.

At 1908, registration markers are optionally identified in the 2D image(s). Examples of registration markers include external radiopaque markers, vertebra (or other bones), contours of the heart and/or particular anatomical landmarks such as valves and the RV wall. Additional registration data which may be acquired (or controlled) includes the orientation of the imaging machine probe relative to the patient body and/or bed coordinate systems.

At 1910 a registration of the 2D image to the 3D data may be generated, for example, using the registration markers and/or, at least as a starting point, imaging system orientation. In some embodiments of the invention, this registration defines a first transformation or mapping between the fluoroscopic image and the CT data. Such registration is optionally based on an algorithm that finds a best or satisfactory (e.g., with respect to error) matching between the co-location of the image registration markers (for example matching vertebrae) of the pre procedure volumetric image or data and the intra procedure image. Optionally, this first registration/mapping is adjusted when the imager is moved from a known angulation to a new angulation.

At 1912, a probe or other tool in the body is identified in the 2D image. In some embodiments of the invention, the identification is based on silhouette and/or radio-opaque section(s) and/or marker(s) thereon, for example, for a pacing lead, electrodes.

As (if) the 2D image is a projection image, the exact location in space of the tool relative to the 3D coordinates cannot generally be known from the 2D location of the tool on the image, possibly even if the distance and magnification of the imager are known (and/or at least not to a desired precision).

In some embodiments of the invention, one or more interactions between the tool and anatomical features which position is known (e.g., due to the first registration/mapping) is used. In one example, an interaction is identified (1914), for example, between a tip of a lead and the RV septal wall. This can be detected, for example, based on resistance, deforming of the lead (or sheath and/or electrogram. The detection can be automatic by the system or manually and reported by an operator, to the system. An image may be captured when such contact is detected, for example, by the system signaling image capturing circuitry. Once identified, this interaction limits the possible locations where the tool can be. In some embodiments of the invention, more than one interaction is used, for example, one or more locations where the tool leans against tissue (e.g., the tricuspid annulus) is used.

At 1916 a set of possible locations for the tool is optionally generated. The set may be larger, for example, if computed before the contacting and contracted using the contacting, or smaller, for example generated after the contracting.

At 1918, an exact location may be determined, for example, base done the above constraints or on an additional constraint, for example, that the catheter is restricted by its flexibility and entrance point to the RV. This location can be converted into a second registration/mapping, back from the fluoroscopic image to the CT/volumetric image.

In general, the system has two complementary units of information: 1. The 2D location of the indwelling tool on the intra procedure viewer monitor (e.g., based on the first registration mapping which using the transformation that was formed in the CT to fluoroscopy step) correspond to a line of possible positions within the patient body or conversely in the patient pre procedure volumetric image.

The system can detect where the line intersects the position of the RV septum (or other anatomical marker), to identify a 3^{rd} dimension of location of the tool.

At 1920 one or more views can be generated (e.g., 2D and/or 3D) from the 3D data, using annotation on the data such as conduction system data and/or back projections from the fluoroscopic image. In particular, the views may be generated using a DRR (Digitally Reconstructed Radiograph) technique.

In some embodiments of the invention, the layout of the sheath (or at least its tip) in the RV is calculated using the point of entry into the heart and electrode locations from the fluoroscopic image. The following method is optionally used:
(a) Compute the registration between the CT and the current fluoroscopic image. Optionally, this registration is calculated using anatomical structures such as spinal vertebrae the diaphragm and/or the heart outline. These structures can generally be detected in fluoroscopic images their location in a CT image can also be extracted, for example, using templates followed by pattern matching or other feature identification methods.

It should be noted that in some embodiments of the invention, registration can be thought of as a function F(x,y,z)->(I,j), where (x,y,z) is a point in CT coordinates and (I,j) is a point in fluoroscopic image. One direction of registration is unambiguous-given a point (x,y,z) one can compute the point (I,j). The other direction of registration is not unambiguous but can still be useful: given a point (I,j) one can compute a straight line in CT coordinates with all locations which are mapped to (I,j).

It is noted that this registration method may also be used for the above registration at the start of a procedure. It is noted that the actual c-arm angulations provide a starting point for this registration, as a range of possible angles, assuming a generally prone (or otherwise positioned) patient.
(b) In some embodiments of the invention, it is assumed (e.g., based on a physician reporting) that the distal electrode (e.g., screw) of the pacemaker lead is in contact with RV endocardium, specifically with the septum wall. The surface of RV septum is known from CT data. Given the location (I0,j0) of the screw in the fluoroscopic image, and using the registration from (a) one can construct the straight line of all locations in CT coordinates which are mapped to (I0,j0). The intersection of this straight line and the RV septum surface can be assumed to be the location of the screw in 3D.

Other methods of detecting septum contact can be used and/or a combination thereof, for this and other uses. For example, deformation of the sheath may be detected by image processing of a fluoroscopic image, which deformation is due to advance against the septal wall. In another example, when a lead contacts the septum, an electrogram can be detected. In another example, the lead measures an impedance which changes markedly when the lead is contact with muscle (as opposed to blood). Electrograms and impedance may also be used to detect penetration (e.g., of helix) into the septum, due to changes thereof.
(c) To compute the location of the proximal electrode in 3D, it is optionally assumed that the distance D in mm between the proximal and distal electrodes is known and fixed. Given the point (i1,j1)—the location of the proximal electrode in the fluoroscopy image one can construct a straight line in 3D with all possible locations of the proximal electrode in 3D. There will be two points on this line which are at distance D from the 3D location of the screw computed in (b). Optionally, one of these possibilities is eliminated based on an understanding of possible orientations of the sheath. Specifically, in some embodiments of the invention, the location of entry into the heart (and/or other anatomical constraints such as papillary muscles and locations of RV walls) and general properties of resilience of the sheath or lead are used to calculate possible layouts of the sheath between the entry point and the septum under the constraints that the two electrodes must be in the known locations. After implantation, there is another constraint of fixed angle of the tip do to ventricular tissue engagement. Optionally alternatively or additionally, if the lead is in a sheath, the sheath constrains the layouts as well. This can result in generating an entire layout for the lead/tool and/or at least for a tip of the lead.

In some embodiments of the invention, once the lead layout is known, it can be presented as an overlay on the DRR. Before contacting the septum, the layout may be based on the sheath geometry and entry point into the heart and/or anatomical shape of the heart, which constraints layout. However, the marking may show the placement as estimated.

FIG. 18A shows a transverse view DRR image showing a tool layout of a head of lead (shown as annotation 1802) in the RV with conduction system annotations 1804 (e.g., the LBB) and optional anatomical annotations 1806 (e.g., the septal walls), in accordance with some embodiments of the invention. It is noted that a transverse view is one which physicians cannot generally generate using fluoroscopy but it shows the trajectory through the septum to great benefit. It is noted that DRR methods can be used to generate a selectively enhanced DRR, for example, enhancing anatomical boundaries which might be less visible on a natural fluoroscopic image.

FIGS. 18B-18D are synthetic view sets at various stages of septum penetration, in accordance with some embodiments of the invention. While three views are shown, a different number, such as 1, 2 (e.g., perpendicular), 4 or more may be used.

At FIG. 18B, three DRR views, from different angulations, are shown when the lead is only contacting a septum (shown as annotation 1806).

At FIG. 18C, the same three DRR views are shown when lead 1802 is partly penetrating the septum on its way to the LBB (shown as annotation 1804).

At 18D, the same three DRR views are shown when lead 1802 is at the target LBB, as can be seen by the overlap of lead annotations 1802 with LBB annotation 1804. It is noted that the lead annotation is shown as not crossing the septal wall annotation. In some embodiments of the invention, another annotation may be shown (not depicted) of the expected range of capture of the electrode.

FIG. 18E shows matching real and synthetic views, in accordance with some embodiments of the invention, with, for example, the annotated real fluoroscopic image corresponding to the lowest shown DRR image. Optionally, the fluoroscopic image is used for verification and/or for observing details not mapped back to the volumetric data set.

FIG. 19B shows a 3D view (as three different views) showing an interventional tool, in accordance with some embodiments of the invention. In some embodiments of the invention, instead of or in addition to a 2D view, what is shown is a 3D synthetic view based on the CT data, optionally including deformation according to cardiac and/or breathing phase. By overlaying the location of the interventional tools on this 3D image, in some embodiments of the invention, the fluoroscopy is used mainly for data collection for the system and/or verification by the operator, while the navigational process is performed on the 3D image.

It is noted that annotations and/or synthetic views may be based on and/or overlaid on an ultrasound image instead of or in addition to x-ray and CT images.

### Sheath

A particular feature of some embodiments of the invention is that the recommended pacing locations and/or trajectories may be more difficult for physicians to achieve than typical RV apex locations.

Some embodiments of the invention provide a sheath which allows an operator to more easily make use of the conduction system data to place a pacemaker lead or a pacemaker.

In some embodiments of the invention, the system recommends a sheath based on the anatomy of the patient and the desired pacing location and/or trajectory. For example, a set of sheaths, for example, between 3 and 15 or 3-9 may be available (e.g., numbered) and the system recommends which number sheath to use. The sheath layout may be shown and/or used to generate synthetic views. If a user selects a different sheath of those recognized by the system or inputs the specific parameters of such sheath, the layout of that sheath may be shown during the procedure.

Optionally or additionally, a sheath may be printed on demand, for example, based on the CT data and the planning stage.

In some embodiments of the invention, a controllable sheath is used. In one example, a sheath such as described in U.S. Pat. No. 11,344,702 or a Vizaramed sheath, is used. Optionally, this sheath is modified, for example bending angles/controls made more accurate and/or diameter reduced to be suitable for subclavian pacemaker implantation. In use, the planning stage optionally outputs recommended angular settings for such a sheath (and/or a recommendation of which sheath to select). During a procedure, a physician can optionally indicate a new target location and the system optionally generates a new recommendation for angular settings.

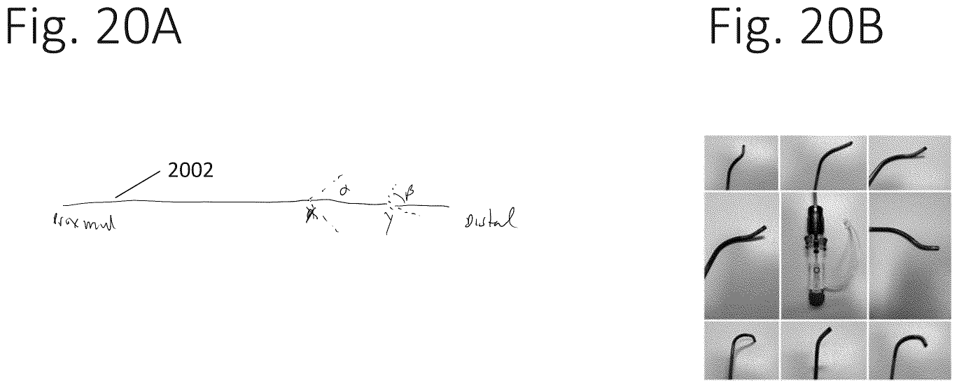

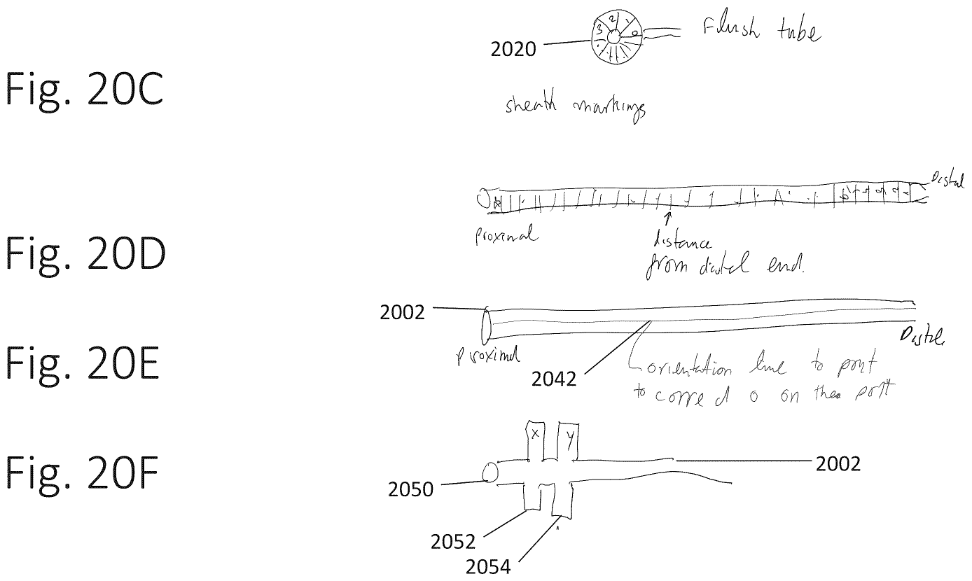

FIG. 20A shows a schematic of a sheath 2002, including two bending locations marked X and Y. The planner optionally generates values of alpha and beta for bending at such locations. While the sheath is shown as straight, in some embodiments of the invention, the sheath is pre-curved. In some embodiments of the invention, the planner also generate axial positing values (or a range of such values, as a guideline).

FIG. 20B is a picture of a two-location deformable sheath, which can be sheath 2002 in various deformation states, in accordance with some embodiments of the invention.

FIG. 20C is a schematic showing of a port face of a port 2020 for use with a sheath (adjustable e.g., sheath 2002 or not adjustable) in accordance with some embodiments of the invention, the port face includes indications which allow an operator to rotate the sheath relative to the port. This may be useful for following suggested sheath rotational positioning instructions provided by the system and/or to control relative rotations during a procedure. In the example shown, a flush tube (optional) is used to set the “zero” angle.

FIG. 20D is a schematic side view of a sheath (adjustable e.g., sheath 2002 or not adjustable) showing axial markings in accordance with some embodiments of the invention. This may be useful for following suggested sheath axial positioning instructions provided by the system and/or to control axial movement during a procedure.

FIG. 20E is another schematic side view of a sheath (adjustable e.g., sheath 2002 or not adjustable) showing an axially extending orientation line 2042 in accordance with some embodiments of the invention. In use, line 2042 may be used to indicate a rotation relative to the port face of FIG. 20C.

FIG. 20F is a schematic showing of a handle 2050 for sheath 2002 showing controllers for two bend locations, in accordance with some embodiments of the invention. In the example shown, two separate controls 2052 and 2054 are provided for separate control of two bends (e.g., X and Y). In some embodiments, a single 2D control is provided. It is noted that while a sheath may be provided with more than two degrees of freedom, in some embodiments of the invention, only two are provided.

In some embodiments of the invention, the controls are calibrated to be prices. In some embodiments, calibration is by manipulating the controls according to a present value (e.g., provided by the system) and the system detecting on a fluoroscopic image a mapping between actual bending degree and the amount applied by the controller. This mapping may be used when later giving instructions to an operator to move to certain angles.

An example use of sheath 2002 can be as follows:
(a) A port, such as shown in FIG. 20C is introduced to the subclavian vein. As noted, the port can have markers on its face, where in one example the point where a flush tube is connected is marked as zero and sequential numbers are marking parts of the port perimeter going counterclockwise from the port flus tube. In one example there are 12 markers. Optionally the port is rotated to a stable and known location, for example, such that the side flush tube is touching the patient's body surface toward the patient's leg. The sheath is optionally oriented such that the orientation marker (FIG. 20E) shows a given value relative to the port markers.
(b) The sheath is advanced via the subclavian vein toward the SVC and from there to the Right Atrium (RA). Then the operator may inject contrast agent through the flush port into the right atrium. In some embodiments, a fluoroscopy image sequence is acquired (cine sequence). The acquired sequences is optionally segmented and/or otherwise processed for registration to the CT data.
(c) In some embodiments, the tricuspid annulus is marked in the pre procedure CT, and is overlaid over the 2D fluoroscopy. In some embodiments, the operator navigates the leading edge of the sheath to the tricuspid annulus plane shown on the fluoroscopy. Optionally, the operator takes note of the distance of insertion of the sheath from the port by reading the value of the insertion distance marker printed on the sheath. This value may be called the “Tricuspid Crossing” and is optionally reported to the system. It is noted that this helps define the distance between the tricuspid crossing (contact with the annulus) and contact with the septum, which may be used or calculating a sheath layout, for example, as described herein.
(d) Next, the operator optionally sets the bending of the proximal joint of the sheath and advances the sheath a first distance (D1), for example, as provided by the system. In some embodiments of the invention, this angle and this distance are personalized values calculated by the system and or the operator during planning.
(e) Next the operator advances a second distance D2 and sets a second angle of the sheath, e.g., of the distal joint thereof. At this time contact with the septum is optionally expected and/or optionally sought (e.g., by advancing the sheath past distance D2. The second angle may be varied of the operator scans a portion of the septum.

This process optionally allows an operator to reach a desired point on the septum (or other part of the heart) and at a desired approach angle.

In some embodiments, the two angles and the two distances (and/or the identity of the sheath) are determined by the system using reverse kinematic methods, after an operator indicates (e.g., during a planning phase) what is the desired contact location and angle of approach, and/or what is the target on the far side of the septum.

In some embodiments, the system outputs during planning a prescription for the operator to reach a target on the far side of the septum (or other location in the heart). The prescription optionally includes angulations (for the two bends), distances (D1 and D2) and an identity of a sheath to use. If the operator uses a different sheath, the prescription may be changed, or the procedure indicated as not possible. It is noted that changing a sheath may change the location of contact with the septum. The sheath optionally includes one or more radio-opaque markers (and/or an enclosed lead may include them) and these markers are optionally identified by the system on a fluoroscopic image and using a known geometry of the sheath, be used to verify sheath identify, track progress and/or suggest new values. For example, a physician may reject a septum location after reaching the septum due to a perceived lack of stability and/or interaction with chordae of the sheath and/or for other reasons. This may require new angulations and/or distances to be suggested. Optionally the angulations and/or distances are provided by the system optionally with a recommended order of application.

After verification, the patient may be treated, e.g., deploy a leadless pacemaker or complete lead implantation and attachment to a pacemaker. Typically, the procedure can now be completed.

### Monitoring and Follow-Up

A particular feature of some embodiments of the invention is the precision at which a pacing lead can be placed and/or expected results simulated. In some embodiments of the invention, after the procedure is completed, the results of the planning and/or of the actual treatment are used to predict an expected time line for the patient. If the patient does not follow the time line, even if the patient is within normal parameters, this may indicate to a treating physician that something is amiss. Optionally alternatively or additionally, the time line may include tests to be made and expected measurements to detect and/or potential signs of disorder. For example, at a certain time there may be a risk of a fibrosis cap interfering with pacing (e.g., based on actual placement and/or pacemaker settings. A patient may be tested at this time to ensure that failure is not occurring.

In some embodiments of the invention, the treatment includes programming a pacemaker (or other implant) with parameter values, or parameter sets or allowed ranges based on the planning and/or the procedure. For example a range of intensities may be selected that are expected to capture, but not expected to cause side effects. The treating physician may then select one of these preprogrammed values, when fine-tuning treatment for the patient. Optionally or additionally, such values are stored off of the pacemaker, for example, in a database associated with the patient and/or pacemaker ID.

### Atrial Pacing

The above description of conduction system pacing has focused on pacing the ventricles. In addition or instead, atria may be paced as well, using methods described herein. In many cases, the atria is paced from within the right atrial appendage, trusting conduction in the atria and between atria to correctly capture the right and left atria. In some embodiments of the invention, an atrial pacing lead (or leadless pacemaker) is implanted at a conduction system location of the atria. Optionally additionally or alternatively, in some embodiments of the invention, when locating the pacing lead, care is taken to ensure it can sense atrial activity and/or AV node activity.

FIG. 1 shows also Bachmann bundle 132 straddling left and right atria. In some embodiments of the invention, pacing is provided where Bachmann bundle 132 straddles the two atria. This location may also have tissue thickness usable for engagement and avoiding penetrating out of the heart by mistake, for pacing in accordance with some embodiments of the invention.

In some embodiments of the invention, a pacing lead is guided to and placed at the Bachman bundle (identified on the CT data, for example), so as, for example, to ensure simultaneous pacing of left and right atria.

### Some Non-Pacing Examples

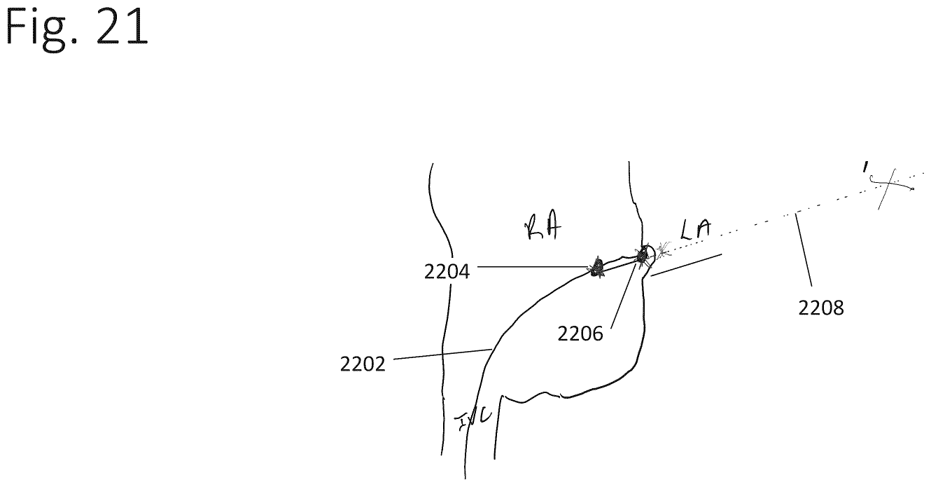

As noted, the indications and mappings and/or other techniques and methods and apparatus described herein can be used for features that are not conduction system and/or for procedures that are not pacing. FIG. 21 is a schematic cross-sectional view of a heart showing a tool, such as a sheath, oriented for trans-septal penetration guided according to the methods of some embodiments of the invention. In this example, markings on the fluoroscopic (or other) image are used for a trajectory to cross the atrial septum. Optionally, the location at which to cross is selected based on anatomical considerations (e.g., in a planning stage). As can be seen two radio-opaque markers 2204 and 2206 on a tool 2202 define a trajectory 2208.

In another example, described in more detail, for example, in the above referenced applications, annotations and/or conduction system data are used to guide an implantation process for an aortic (or other, e.g., tricuspid) valve.

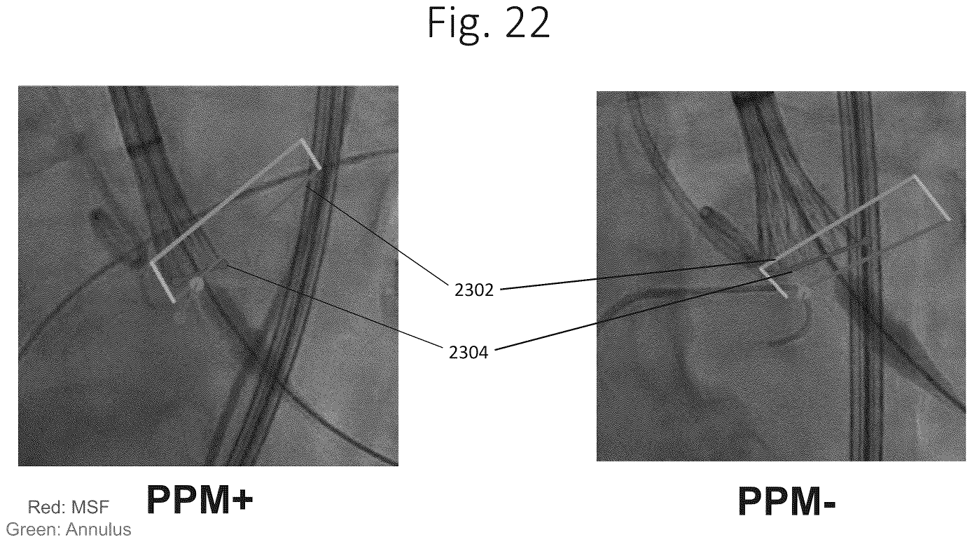

FIG. 22 shows guide annotations for valve implantation, in accordance with some embodiments of the invention, optionally, with a goal of setting depth of implantation so that a need for a pacemaker is avoided or reduced. A trapezoid guide 2302 indicates a danger zone outside and a safe zone inside, where valve expansion may cause damage to a conduction system section 2304. A potentially damaging overlap between the valve and the conduction system is shown in the left image, and absent in the right.

In some embodiments of the invention, these guides are shown not (only) on a fluoroscopic image but also on a synthetic images, for example a 3D image. Such image (and/or projections thereof) can enjoy a mapping back form the 2D fluoroscopic image to the CT data set coordinates. For example, the outline of the valve may be extracted and projected back to the CT image.

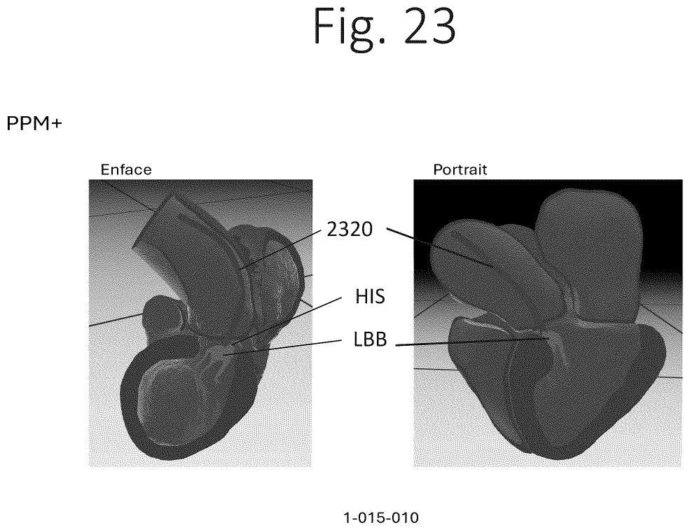

FIG. 23 shows 3D model views of an undesirable interaction between a valve implantation and the conduction system, which may be avoided in accordance with some embodiments of the invention. A delivery tool 2320 is shown overlapping an LBB and/or a His bundle section which suggests that valve delivery using this location will cause pressure on the LBB and potentially cause an LBB block or other conduction problem which might require a pacemaker. In some embodiments of the invention, such planning is used to avoid the need for a pacemaker and/or for allowing preemptive implantation of a pacemaker, optionally with physiological pacing close to the expected location of damage.

In other applications, the conduction system imaging is used to plan positioning of tricuspid replacement implants to spare the conduction system. It is noted that the conduction system can be very close to the tricuspid annulus, for example, within 2-4 mm, so the conduction system is at risk of damage during tricuspid implantation. Similar methods may be used as for sparing of CS damage during aortic valve implantation.

Other structural interventions and especially catheter based device implants may also cause conduction system damage, for example, devices for repair of septal defects and devices for creating a shunt between ventricles or atria. In both cases the device may press against sensitive conduction system components after implantation and/or during implantation (or the delivery system or associated tools may). Knowledge of the location of sensitive conduction system parts can be used to generate guidelines for location which should be avoided and/or for showing safe locations. The target of implantation may also be annotated, for example, on real or synthetic fluoroscopic images and/or on a 3D guiding image.

### General

It is expected that during the life of a patent maturing from this application many relevant electrical system maps of the heart will be developed; the scope of the term conduction system data is intended to include data provided by all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### EXAMPLES

Reference is now made to the following examples, which together with the above descriptions illustrate some embodiments of the invention in a non limiting fashion.

### Example 1

### Experimental Procedures

Continuous and categorical data were compared across groups of interest using non-parametric Kruskal-Wallis statistic and Pearson's Chi-Squared test with continuity correlation adjustment where applicable. Analysis of co-variance (ANCOVA) was performed to compare the response parameters between groups of interest while adjusting for potential confounders. Several multiple linear models were regressed on response parameters at follow-up, while adjusting for baseline values as well as the breadth of available clinical and demographic variables. Final models were selected in the process of backward elimination, in favor of lower Akaike Information Criterion values, better goodness-of-fit (as tested by Hosmer-Lemeshow statistic), higher adjusted R-squared values and residual analysis.

For subgroup analysis of response parameters between heart failure categories, post-hoc Tukey HSD method was applied to the respective ANCOVA models.

The location of the pacing lead was determined by CTA as exemplified in FIG. 24.

The present inventor introduced three new absolute pacing lead location measures from post CSP CTA: (1) the geodesic distance between the pacing lead and the anterior position of the floor of the membranous septum (LC distance), (2) the aerial distance between the pacing lead and the LV endocardium (LE distance) and (3) the geodesic distance from pacing lead to the mid apical part of the interventricular septum (LX distance). It is believed that the following physiologic significance: LC—the distance of the pacing lead from the origin of the left bundle (calculated with its geodesic path through the interventricular septum); LE—the location of the lead within the septum, where LE=0 was set at the LV endocardial surface and negative value refer to the tip of the pacing lead helix located within the interventricular septum and positive LE refer to pacing lead helix located inside the LV cavity; LX—is the distance through the interventricular septum that activation needs to reach to via the conduction system to generate a physiologic normal left ventricle activation. FIG. 25A is a depiction of geodesic distance path from a pacing lead intra ventricular location (green ball) to the anterior aspect of the membranous floor LC. FIG. 25B is a depiction of the aerial distance between the pacing lead location to the LV endocardium; and FIG. 25C is a depiction of PX the geodesic distance to the apical part of the mid interventricular septum.

### Example 2

### Pacing Lead Proximity to the Bundle of his and to the Left Ventricular Endocardium May Affects Outcome of Conduction System Pacing

A total of 180 participants were included in the study, of which 147 (82%) and 33 (18%) were seen at the MADU and VCU tertiary referral centers, respectively. The mean (SD) age was 62.1 (13.3), 54% were female. The indications for conduction system pacing included (in the order of frequency): AV block (39%), ejection fraction of <35% (26%), sick sinus syndrome (16%), ejection fraction range of 35-50% (8%), pacing-induced cardiomyopathy (6%), and AV node ablation (4%). Participants were categorized as having HFpEF (50%), HFmEF (11%), and HFrEF (38%) based on the baseline ejection fraction. Mean (SD) baseline LVEF was 48.1% (15.4); LVESV, LVEDV, LVESD, and LVEDD were 79.7 mL (57.0), 135.8 mL (62.4), 31.0 mm (5.1) and 51.2 mm (9.3), respectively. The frequency of NYHA clinical heart failure severity classes II, III, and IV were 48%, 45%, and 7%, respectively. Based on CTA measurements, the mean (SD) geodesic distance of lead tip from conduction system was 28.3 mm (7.8), and the mean (SD) aerial distance from endocardium was 0.3 mm (SD 2.9). The exact location of the lead tip with reference to the conduction system constituents (i.e., Trunk vs Fascicles, vs Myocardium) was possible to ascertain for 80% of cases. Accordingly, participants were found to have received pacing at the Trunk (13%), Fascicles (47%), and Myocardium (21%). The comprehensive clinical and demographic characteristics of study participants are presented in Table 1, below.

TABLE 1
Clinical and Demographic Characteristics of Study Sample

| | Trunk | | Fascicles | | Myocardium | | Total | | |
|---|---|---|---|---|---|---|---|---|---|
| | (n = 23) | | (n = 84) | | (n = 37) | | (n = 180) | | |
| | Mean/NSD/% | | Mean/NSD/% | | Mean/N SD/% | | Mean/NSD/% | | P-value |
| Age | 62.6 | 10.9 | 63.4 | 12.7 | 64.5 | 12.6 | 62.1 | 13.3 | 0.7312 |
| Gender (Female) | 8 | 35% | 42 | 50% | 19 | 51% | 97 | 54% | 0.0645 |
| Heart Failure | | | | | | | | | 0.0001*** |
| HFpEF | 5 | 22% | 44 | 52% | 19 | 51% | 90 | 50% | |
| HFmEF | 3 | 13% | 8 | 10% | 4 | 11% | 20 | 11% | |
| HFrEF | 15 | 65% | 31 | 37% | 13 | 35% | 68 | 38% | |
| NYHA | | | | | | | | | 0.1509 |
| II | 7 | 30% | 44 | 52% | 14 | 38% | 86 | 48% | |
| III | 14 | 61% | 38 | 45% | 17 | 46% | 81 | 45% | |
| IV | 2 | 9% | 2 | 2% | 6 | 16% | 13 | 7% | |
| Baseline Echo-CG | | | | | | | | | |
| LVEF (%) | 39.2 | 15.2 | 48.9 | 14.7 | 48.6 | 15.8 | 48.1 | 15.4 | 0.0358* |
| LVESV (mL) | 118.7 | 61.7 | 73.9 | 47.9 | 88.8 | 68.1 | 79.7 | 57.0 | 0.015* |
| LVEDV (mL) | 175.7 | 69.2 | 129.2 | 50.6 | 146.6 | 79.8 | 135.8 | 62.4 | 0.0372* |
| LVESD (mm) | 27.0 | 2.8 | 31.9 | 5.0 | 30.4 | 5.6 | 31.0 | 5.1 | 0.3627 |
| LVEDD (mm) | 56.6 | 11.5 | 50.6 | 8.1 | 51.5 | 9.9 | 51.2 | 9.3 | 0.0933 |
| Pierce Sign | 19 | 83% | 49 | 58% | 0 | 0% | 68 | 38% | 0.0002*** |
| Non-specific Septal Pattern | 3 | 13% | 22 | 26% | 9 | 24% | 37 | 21% | 0.0000*** |
| LBB Potential | 12 | 52% | 49 | 58% | 23 | 62% | 112 | 62% | 0.1049 |
| Indications | | | | | | | | | |
| AV block | 5 | 22% | 36 | 43% | 9 | 24% | 70 | 39% | 0.3919 |
| AV node ablation | 1 | 4% | 4 | 5% | 2 | 5% | 8 | 4% | 0.1704 |
| Sick Sinus Syndrome | 3 | 13% | 11 | 13% | 9 | 24% | 28 | 16% | 0.0018** |
| Pacing-induced CMP | 1 | 4% | 5 | 6% | 2 | 5% | 10 | 6% | 0.4834 |
| EF <35 | 12 | 52% | 20 | 24% | 9 | 24% | 47 | 26% | 0.0002*** |
| EF 35-50 | 2 | 9% | 7 | 8% | 3 | 8% | 14 | 8% | 0.0649 |
| Geodesic L-C (mm) | 22.2 | 6.6 | 27.3 | 6.3 | 34.2 | 7.9 | 28.3 | 7.8 | 0.0000*** |
| Aerial L-E (mm) | 0.8 | 2.1 | 0.8 | 2.7 | −1.0 | 3.3 | 0.3 | 2.9 | 0.0140* |
| Follow-up Echo-CG | | | | | | | | | |
| LVEF (%) | 54.2 | 12.2 | 57.1 | 9.3 | 53.6 | 12.2 | 55.7 | 10.5 | 0.3768 |
| LVESV (mL) | 62.8 | 39.9 | 49.1 | 20.8 | 71.4 | 64.7 | 55.3 | 40.1 | 0.3979 |
| LVEDV (mL) | 127.5 | 41.0 | 112.3 | 27.7 | 133.2 | 78.1 | 116.6 | 48.7 | 0.5928 |
| LVESD (mm) | 24.5 | 4.9 | 33.2 | 10.1 | 59.6 | 85.4 | 41.6 | 50.7 | 0.4523 |
| LVEDD (mm) | 50.0 | 7.3 | 48.0 | 6.2 | 60.1 | 65.4 | 50.4 | 29.6 | 0.8106 |
| Follow-up duration (weeks) | 112.8 | 45.4 | 98.8 | 60.7 | 84.0 | 60.2 | 95.4 | 61.2 | 0.3010 |

Continuous variables compared using Kruskal-Wallis test statistic
Occurrence frequencies compared using Pearson's Chi-Squared test with continuity correlation adjustment where applicable.

Compared to participants with pacing leads at any of the fascicles or the myocardium, participants who received pacing at the trunk included slightly fewer women (35% v 50% v 51%, p=0.0645), were more often categorized as having HFrEF (65% v 37% v 35%) and less often as HFpEF (22% v 52% v 51%, p=0.0001); accordingly had on average lower baseline LVEF (39.2% [SD 15.2] v 48.8% [SD 14.7] v 48.6% [SD 15.8], p=0.0358) as well as larger end-systolic and end-diastolic volumes (p=0.015 and p=0.0372, respectively; above). Sick sinus syndrome was a more common indication among participants who received myocardial pacing (24% v 13% v 13%, p=0.0018), whereas Ejection fraction of below 35% was more often the indication for Trunk-paced participants (52% v 24% v 24%, p=0.0002). The ‘Piercing sign’ was apparent more often among trunk-paced participants, compared to fascicle- and myocardium-paced cases (80% v 58% v 0%, p=0.0002), while the non-specific septal pattern was seen less often (13% v 26% v 24%, p<0.0001). There were no significant differences between groups paced at different sites with regard to age, NYHA categories, presence of LBB potential, duration of the follow-up, or the frequency of remaining indications, including AV block, AV nodal ablation, pacing-induced CMP, and ejection fraction in the range of 35-50%.

The geodesic distance of lead tip (L) from the conduction system (C) was significantly shorter for the group paced at the trunk (22.2 mm, SD 6.6) compared to the group paced at the fascicles (27.3 mm, SD 6.3), which in turn was shorter compared to the group with myocardial-pacing (34.2 mm, SD 7.9; p<0.0001). Additionally, the aerial distance of the lead tip (L) from left ventricular endocardium (E) was significantly longer for trunk—(0.8 mm, SD 2.1) and fascicles-paced groups (0.8 mm, SD 2.7), compared to myocardium-paced cases (−1.0 mm, SD 3.3; p=0.014; FIG. 26A and FIG. 26B and above).

Over an average followed-up duration of 95.4 weeks (SD 61.2), participants paced at the Trunk experienced significantly larger absolute increase in LVEF (15.0% [SD 15.0]) compared to fascicle—(8.7% [SD 14.4]) and myocardium-paced groups (6% [SD 11.5]; p=0.00132), as well as a larger absolute decrease in LVESV (−55.9 mL [SD 51.7] v −24.9 mL [SD 45.6] v 17.4 mL [SD 25.9]; p<0.0001), and a larger absolute decrease in LVEDV (−48.2 mL [SD 56.1] v −16.9 mL [SD 48.4] v −13.4 mL [SD 31.1], p=0.0011), while adjusted for baseline LVEF and LVEDD. The relative (%) change followed a similar pattern ( ).

In a linear model adjusted for baseline LVEF, for every 1 mm decrease in geodesic L-C distance, follow-up LVEF increased by 0.36% (SE 0.11, p=0.0010). When additionally adjusted for an aerial L-E threshold of >−2.5 mm, shorter geodesic L-C increased follow-up LVEF by 0.28% (SE 0.11. p=0.0135) for every 1 mm, while aerial L-E of greater than −2.5 mm increased follow-up LVEF by 5.58% (SE 2.43, p=0.0234; FIG. 27 and Table 3.

TABLE 2
Change in echocardiographic parameters at follow-up.

| | Trunk | | Fascicles | | Myocardium | | Total | | |
|---|---|---|---|---|---|---|---|---|---|
| | (n = 23) | | (n = 84) | | (n = 37) | | (n = 180) | | |
| | Mean | SD | Mean | SD | Mean | SD | Mean n | SD | ^{†}P-value |
| Absolute Change | | | | | | | | | |
| LVEF (%) | 15.0 | 15.0 | 8.7 | 14.4 | 6.0 | 11.5 | 9.1 | 14.0 | 0.00132 ** |
| LVESV (mL) | −55.9 | 51.7 | −24.9 | 45.6 | −17.4 | 25.9 | −28.8 | 44.8 | 0.00007 *** |
| LVEDV (mL) | −48.2 | 56.1 | −16.9 | 48.4 | −13.4 | 31.1 | −21.8 | 47.9 | 0.00110 ** |
| LVESD (mm) | −2.5 | 2.1 | −0.7 | 7.1 | 33.6 | 90.0 | 11.1 | 53.6 | 0.33385 |
| LVEDD (mm) | −6.5 | 8.3 | −2.9 | 7.5 | 8.6 | 66.4 | −0.7 | 33.6 | 0.17321 |
| Relative (%) Change | | | | | | | | | |
| LVEF (%) | 52.0 | 52.7 | 29.8 | 48.1 | 19.8 | 33.8 | 31.2 | 46.7 | 0.00043 *** |
| LVESV (mL) | −38.3 | 32.3 | −17.7 | 42.0 | −18.3 | 31.2 | −21.5 | 38.7 | 0.04638 * |
| LVEDV (mL) | −19.9 | 28.9 | −5.8 | 31.3 | −9.1 | 21.2 | −9.1 | 29.1 | 0.09678 |
| LVESD (mm) | −9.7 | 8.9 | −2.1 | 22.3 | 100.5 | 273.2 | 33.1 | 162.6 | 0.34191 |
| LVEDD (mm) | −9.6 | 13.5 | −4.4 | 13.9 | 18.3 | 135.2 | 0.3 | 67.9 | 0.21328 |

^{†}Analysis of co-variance adjusted for baseline LVEF (%) and LVEDD (mm)

TABLE 3
Multiple linear models regressed on follow-up LVEF (%)

| | Estimate | SE | t | p |
|---|---|---|---|---|
| Model 1 | | | | |
| (Intercept) | 51.02 | 3.92 | 13.00 | 0.0000*** |
| Geodesic L-C (mm) | −0.36 | 0.11 | −3.36 | 0.0010** |
| Baseline LVEF (%) | 0.31 | 0.05 | 6.01 | 0.0000*** |
| Model 2 | | | | |
| (Intercept) | 43.22 | 5.15 | 8.40 | 0.0000*** |
| Geodesic L-C (mm) | −0.28 | 0.11 | −2.51 | 0.0135* |
| Aerial L-E >−2.5 (mm) | 5.58 | 2.43 | 2.29 | 0.0234* |
| Baseline LVEF (%) | 0.33 | 0.05 | 6.36 | 0.0000*** |
| Model 3: LVESV | | | | |
| (Intercept) | −28.76 | 27.03 | −1.06 | 0.2898 |
| Baseline LVESV (mL) | 0.53 | 0.08 | 6.56 | 0.0000*** |
| Geodesic L-C (mm) | 0.87 | 0.41 | 2.10 | 0.0378* |
| Aerial L-E >−2.5 (mm) | −11.54 | 12.05 | −0.96 | 0.3406 |
| Baseline LVEF (%) | 0.62 | 0.31 | 2.02 | 0.0460* |
| Model 4: LVEDV | | | | |
| (Intercept) | 12.44 | 29.75 | 0.42 | 0.6767 |
| Baseline LVEDV (mL) | 0.51 | 0.07 | 7.44 | 0.0000*** |
| Geodesic L-C (mm) | 0.87 | 0.49 | 1.80 | 0.0749 |
| Aerial L-E >−2.5 (mm) | −0.58 | 14.11 | −0.04 | 0.9673 |
| Baseline LVEF (%) | 0.28 | 0.29 | 0.98 | 0.3297 |
| Model 5: LVESD | | | | |
| (Intercept) | 419.61 | 169.65 | 2.47 | 0.0258* |
| Baseline LVESD (mm) | −1.04 | 2.68 | −0.39 | 0.7040 |
| Geodesic L-C (mm) | −0.82 | 1.61 | −0.51 | 0.6160 |
| Aerial L-E >−2.5 (mm) | −36.03 | 20.80 | −1.73 | 0.1037 |
| Baseline LVEF (%) | −5.00 | 1.71 | −2.93 | 0.0104* |
| Model 6: LVEDD | | | | |
| (Intercept) | 78.52 | 35.80 | 2.19 | 0.0302* |
| Baseline LVEDD (mm) | 0.17 | 0.42 | 0.41 | 0.6844 |
| Geodesic L-C (mm) | −0.16 | 0.40 | −0.41 | 0.6839 |
| Aerial L-E >−2.5 (mm) | −23.45 | 9.30 | −2.52 | 0.0130* |
| Baseline LVEF (%) | −0.23 | 0.26 | −0.91 | 0.3638 |

It is suggested that shorter L-C distances and L-E values >−2.5 mm are associated with optimal cardiomechanical responses.

These findings were corroborated through the longitudinal evaluation of patients' echocardiographic parameters and clinical outcomes during follow-up. In subsequent multiple linear models, shorter geodesic L-C distances significantly decreased follow-up LVESV (by 0.87 mm [SE 0.41] for every 1 mm; p=0.0378), and decreased LVEDV, albeit at a trend level (by 0.87 mm [SE 0.49], p=0.0749), while aerial L-E distances >−2.5 mm significantly reduced LVEDD (by −23.5 mm [SE 9.3], p=0.013); all models were adjusted for the same echocardiographic parameter at baseline, as well as baseline LVEF.

### Subgroup Analysis

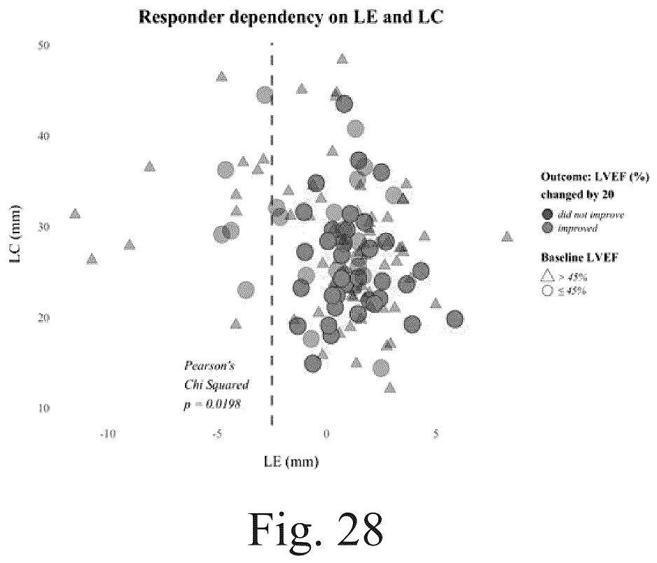

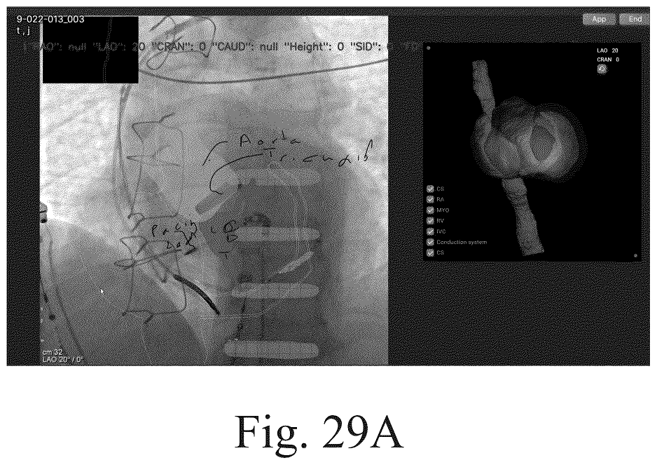

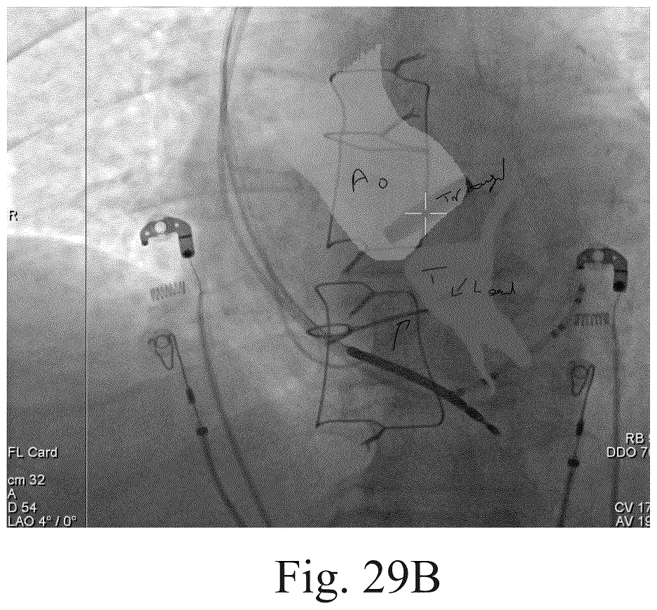

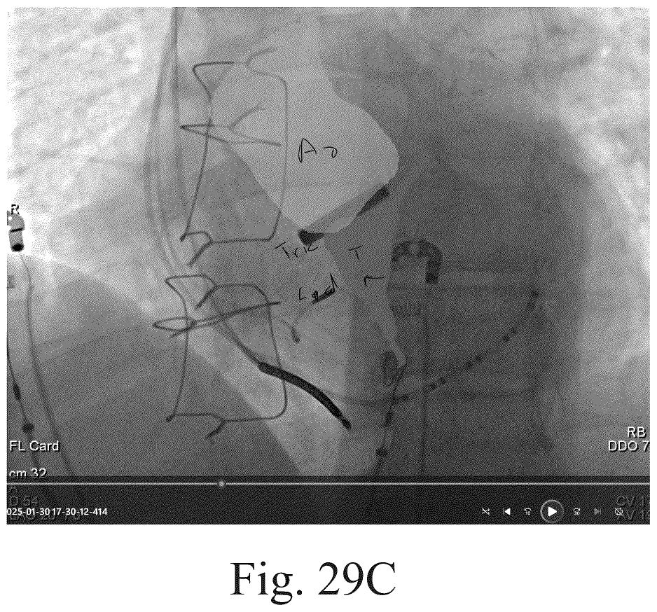

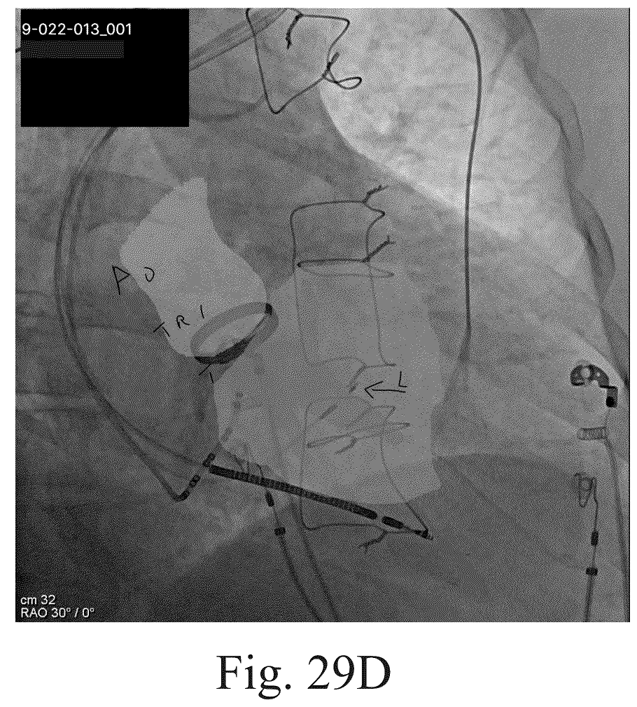

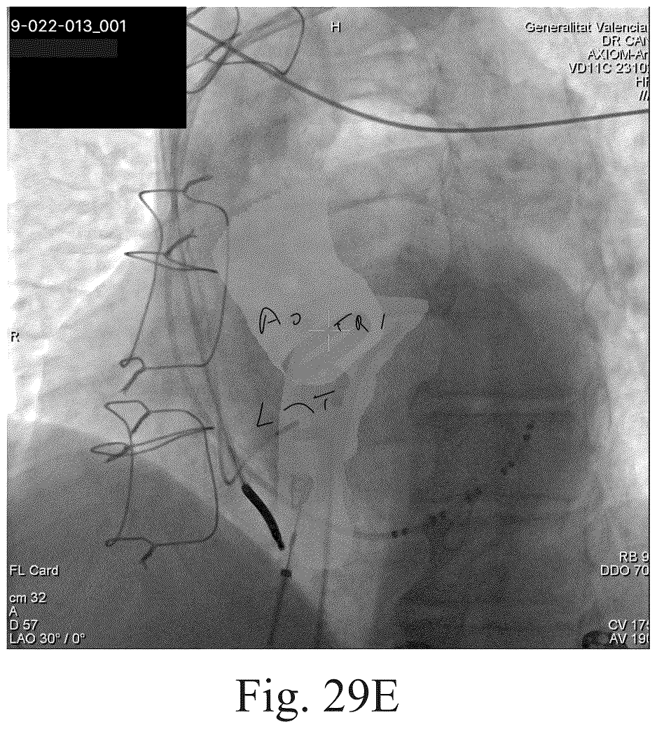

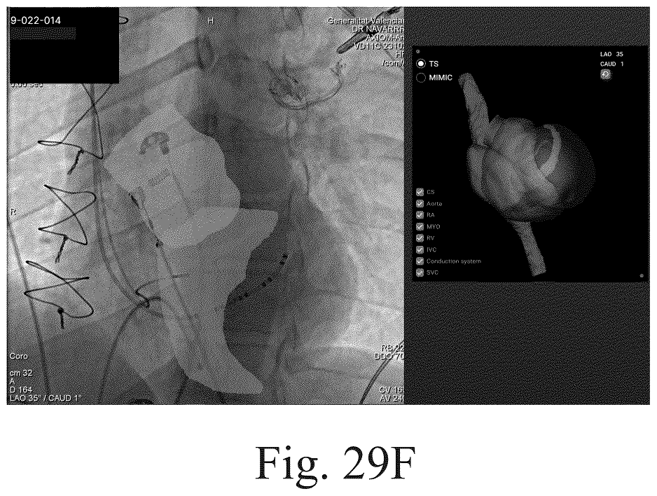

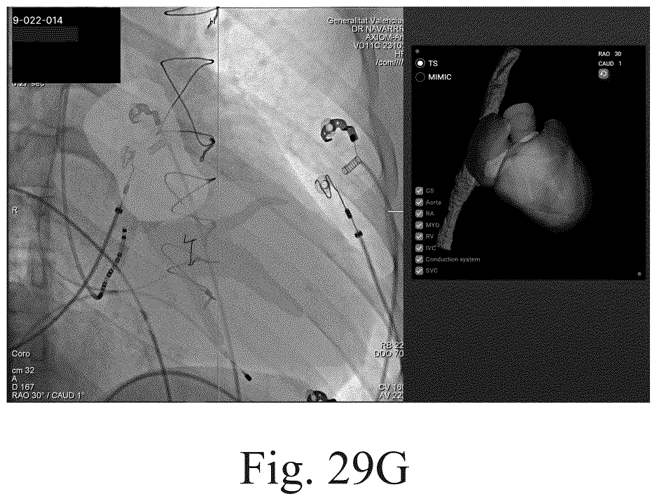

In a subgroup analysis of the three ejection-fraction-based heart failure categories adjusted for geodesic L-C and aerial L-E distances, participants with HFrEF experienced the largest relative increase in LVEF (mean 63.8%, 95% CI [53.1; 74.4]), while relative (%) LVEF change was not significant for HFmEF (9.9%, 95% CI [−8.4; 28.2]) and HFpEF (−5.8%, 95% CI [−15.4; 3.8]; below and FIG. 28). Post hoc analysis using Tukey HSD showed that compared to participants with HFpEF and HFmEF, those categorized as HFrEF experienced significantly larger relative (%) increase in LVEF (p<0.001 for both post-hoc pairs;

TABLE 4
Subgroup analysis of heart failure ejection fraction categories

| Echocardiographic | | | | | |
|---|---|---|---|---|---|
| Parameter | Mean^{†} | SE | df | 95% CI | |
| Relative (%) LVEF change | | | | | |
| HFrEF | 63.8 | 5.4 | 128 | 53.1 | 74.4 |
| HFmEF | 9.9 | 9.2 | 128 | −8.4 | 28.2 |
| HFpEF | −5.8 | 4.8 | 128 | −15.4 | 3.8 |
| Relative (%) LVEDV change | | | | | |
| HFrEF | −22.2 | 6.0 | 104 | −34.1 | −10.3 |
| HFmEF | −1.1 | 10.9 | 104 | −22.6 | 20.5 |
| HFpEF | −1.2 | 6.6 | 104 | −14.2 | 11.8 |
| Relative (%) LVESV change | | | | | |
| HFrEF | −40.7 | 7.2 | 104 | −55.0 | −26.3 |
| HFmEF | −9.9 | 13.1 | 104 | −35.9 | 16.2 |
| HFpEF | 3.9 | 7.9 | 104 | −11.8 | 19.5 |

| Tukey HSD post-hoc | Mean | | | t | |
|---|---|---|---|---|---|
| contrast | Difference^{†} | SE | df | ratio | p |
| Relative (%) LVEF change | | | | | |
| HFrEF v HFpEF | 69.5 | 5.8 | 128 | 12.0 | <0.0001 |
| HFrEF v HFmEF | 53.8 | 9.7 | 128 | 5.57 | <0.0001 |
| HFmEF v HFpEF | 15.7 | 9.6 | 128 | 1.63 | 0.2350 |
| Relative (%) LVESV | | | | | |
| change | | | | | |
| HFrEF v HFpEF | −44.5 | 6.89 | 104 | −6.46 | <.0001 |
| HFrEF v HFmEF | −30.8 | 12 | 104 | −2.56 | 0.0319 |
| HFmEF v HFpEF | −13.7 | 12.4 | 104 | −1.11 | 0.5124 |
| Relative (%) LVEDV | | | | | |
| change | | | | | |
| HFrEF v HFpEF | −21.0 | 5.71 | 104 | −3.68 | 0.0011 |
| HFrEF v HFmEF | −21.1 | 9.99 | 104 | −2.12 | 0.0913 |
| HFmEF v HFpEF | 0.10 | 10.3 | 104 | 0.01 | 1.0000 |

^{†}adjusted for geodesic L-C distance (mm) and aerial L-E distance >−2.5 mm.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of a machine-assisted cardiac pacing electrode placement procedure in a patient in need thereof, comprising:
(a) identifying a target location using a 3D data set of a heart of said patient, said target location being selected according to a desired cardio mechanical parameter for a statistically significant pacing efficacy, said 3D data set having been acquired using data collection from outside the heart; and
(b) providing machine-assisted guidance of a tool to said target location.

2. The method of claim 1 comprising receiving said 3D data set of said heart of said patient by a computer-based guidance system prior to (a); and/or
receiving a setting for Hierarchical Physiologic Pacing (HPP) on said computer-based guidance system prior to receiving said 3D data set.

3. The method of claim 1, wherein said target location is identified based on anatomical landmarks or wherein said target location describes a desired position of a lead tip of said pacing electrode with respect to said anatomical landmarks.

4. The method of claim 1, wherein said target location is defined by a geodesic distance between said lead tip to an anterior position of the floor of the membranous septum (LC) and an aerial distance (LE) between said lead tip and left ventricular endocardium.

5. The method of claim 4, wherein:
said LC is below 30 mm; and/or
said lead tip is not more than 2.5 mm away from the left ventricular endocardium (LE>−2.5 mm).

6. The method of claim 5, wherein said desired cardio mechanical parameter for a statistically significant pacing efficacy is dependent on a heart condition of said patient.

7. The method of claim 6, wherein said cardio mechanical parameter is LVEF, and as said LVEF becomes more pathological when said LC is selected shorter, optionally—with each 1 mm decrease in distance corresponding to an increase in LVEF by approximately 0.33%.

8. The method of claim 7, wherein:
said patient is diagnosed with heart failure (HF);
said patient is expected to benefit from reducing LVESV or is selected expected to benefit from reducing LVESV;
said patient is diagnosed with HFrEF;
said patient is diagnosed with HFpEF;
and/or
said patient is expected to benefit from reducing LVEDD or is selected to benefit from reducing LVEDD.

9. The method of claim 1, wherein said cardio mechanical parameter is selected from the group consisting of LVEF, LVESF, LVEDV and LVEDD.

10. The method of claim 9, wherein said cardio mechanical parameter is:
LVEF and said statistically significant pacing efficacy is defined by an absolute increase in LVEF of at least 10%;
LVEDV and said statistically significant pacing efficacy is defined by an absolute decrease in LVEDV of at least 45 mL;
LVESV and said statistically significant pacing efficacy is defined by an absolute decrease in LVESV of at least 2 mm; and/or
LVEDD and said statistically significant pacing efficacy is defined by an absolute decrease in LVEDD of at least 5 mm.

11. The method of claim 1, wherein said guidance comprises: area or point of penetration and penetration angle;
comprises a number of rotations of a lead or delivery system;
a sheath identity, lead identity, pacemaker and/or one or more set of pacing parameters; and/or
one or more instructions or alerts to show during said procedure.

12. The method of claim 1, further comprising supporting planning said procedure on said 3D data set before said procedure by an operator;
receiving said target location comprises simulating an effect of pacing at said target location; and/or
simulating a geometric access to said target location.

13. The method of claim 12, comprising evaluating a normalizing effect of said pacing on cardiac electrical and/or mechanical activity and/or surface ECG and/or electrocardiogram.

14. The method of claim 1, comprising guiding an operator during said procedure.

15. The method of claim 14, wherein said guiding comprises:
marking up one or more fluoroscopic image and/or ultrasonic image;
recommending or applying imager angulations;
generating one or more instructions and/or alerts according to a progress of said procedure;
recommending or applying a delivery system geometric adjustment; and/or
reporting a distance in the septum from a tool tip to an LV wall, the distance being along a trajectory and/or to a nearest wall.

16. An apparatus configured to provide guidance of a pacing electrode, the apparatus comprising:
a memory storing 3D data set of a heart of a patient; and
at least one processor configured for identifying a target location using said 3D data set of said heart of said patient according to a desired cardio mechanical parameter for a statistically significant pacing efficacy.

17. The apparatus of claim 16, wherein said memory stores a cardio mechanical parameter of said patient.

18. The apparatus of claim 16, wherein said at least one processor provides machine-assisted guidance of a tool to said target location.

19. The apparatus of claim 16, wherein said guidance comprises:
planning a pacing electrode guidance procedure on said 3D data set before said procedure by an operator;
simulating an effect of pacing at said target location; and/or
simulating a geometric access to said target location.

20. The apparatus of claim 16, comprising an imager for collecting said 3D data set.
